SEQUENCE LISTING (8 sequences)

SEQ ID NO: 1 - DNA, 946 bp, EPSTEIN BARR VIRUS
ttagaaatttaagagatcctcgtgtaaaacatctggtgtccgggggataatggagtcaacatccaggcttgggcacatctgcttcaacaggaggcgcagcctgtcattttcagatgatttggcagcagccacctgcggacaaaaatcaggcgtttagatggggcatttatgtttgggacgctagccgcctgggcattcgtgttagtatatactgacctcacggtagtgctgcagcagttgcttaaacttggcccggcattttctggaagccacccgattcttgtatcgctttatttctagttcagaatcgcattcctccagctgcgagcaagggaatgcgttactacaagtggtgcctagtcagttgaaacaagccccaccatccgctgccgcccctccatgagccccaccgtccgctgccgcccctccttgagcccctccttaccgattctggctgttgtggtttccgtgtgcgtcgtgccggggcagccactggtgcaggctgtggaacaccaatgtctgctagctgttgtccttggttagccccggggcaagcaaacaccactgctgctgctgtttgaacagtagaattgtctccaggttgaggtgcttctcccccggcttggttagtctgttgattctgggttatgtcggagactgggaacagctgaggtgctgcataagcttgataagcattctcaggagcaggctgaggggcagaaaaccacgacccagtcggagcggttgaaacatgataggcagttagctggccttgtggcagaggctctggcagcaccggccacagcacacaaggcaaaggagcttgcgatggccctcccaggtcctgatagactctggtagcttggtcaaaagcttgtacaaaaggcacctggtatgggtcaggtgtaaattttacatcttcagaagtcgagtttgggtccatcat

SEQ ID NO: 2 - DNA, 21 bp, ARTIFICIAL SEQUENCE
  SYNTHETIC PROBE
gccgcctgggcattcgtgtta

SEQ ID NO: 3 - DNA, 21 bp, ARTIFICIAL SEQUENCE
  SYNTHETIC PROBE
gccgtccgggcattcgtgtta

SEQ ID NO: 4 - DNA, 18 bp, ARTIFICIAL SEQUENCE
  SYNTHETIC FORWARD PRIMER
gcacatctgcttcaacag

SEQ ID NO: 5 - DNA, 18 bp, ARTIFICIAL SEQUENCE
  SYNTHETIC FORWARD PRIMER
gaacagctgcttcaacag

SEQ ID NO: 6 - DNA, 18 bp, ARTIFICIAL SEQUENCE
  SYNTHETIC REVERSE PRIMER
cgtgaggtcagtatatac

SEQ ID NO: 7 - DNA, 18 bp, ARTIFICIAL SEQUENCE
  SYNTHETIC REVERSE PRIMER
cgtaagttcagtatatac

SEQ ID NO: 8 - DNA, 171096 bp, Cercopithecine herpesvirus 15 strain LCL8664
ccccttaacaggggggagggggggttgaggggccgggatggatgttgggggacttcggttctttgctcttaaacctttgcaaaatttttgcgtgccgttttgcacgttcataacgttttcctttggtttcctacaggactatggcgcctggatggctgcccgtggtttctgcaccatatcttttctggctggctggaattgccgcttcctgcttttcagcctccgttagtgctcttgttgttactaccggccttgctctctcactcttgctacttgcggccctggttaactcatatgccgctcagcgaaggaaggccgttactaagttaactatcctcgtcggcattgttacatgtaagtatgtcacccagacagagaacagttgtggaggctcgttgcgcttgcaaccatacgttatccttgtgtattctttcagtttttgccattctcctgacatggattgtccagcctaaaccgagagatgccattgtctttgccttattgaccggggccgctgtactacaggccatttacagtaagtagcagcgtgcttggctcgggggtgtttactgagtaacagggtgctcgactcaggggtgttctgggcacctgaacacagctcacatggtttctcttttctcagtcctagttatgctgctcttcttgctccccacataccggcggagatggcgcaagatgattgtttgcgtcgttctcctgtttttggcctgtgccgtgctcctcgttgtcgatgccatctggcagctgtctcccctactcggatccatgacagtggtagcgctgacactcttgcttttagcattcatcatctggctctcctcaccttggggcataggggccctaggagcagcgcttctaacgctggcggcgggtaagacacagtccgcggatgatgtttgagatgggttgtgtgtttttcagactatgacctaactatgtcctctatgtgcagccttggctctcctgggatcattggtcctcggggaacttaacctggcaacaatgttcctcatcatgttcctgtggactcttggtgagtgcccccttttccacgggtttgtgacttttaacatgtctatatacagaagcctaactatttgtttcttccacagtcattattctcctgtgctcaacattcccacaaactatgtacctcatgtctcggtggctgctgtatgctctgtcccttttattgctcgcctctgcgctcttggccggtggcagcattctccaaacctggggcggagacaacactgagtttttcccgagtgagtagatgttcatgagcctgtacggtgcgcgccgagcatagaaaattaccacccatgggagagtaacaacccctgttattacaggcttgttctgcatgttgctgctcatcgttgccggcattctcttcatcttggctatcctgaccgagtggggcactggaagtaagacatacgggccggtgttcgtctgtctcagtggcctcctgaccatggcagccggcctcgtttgggtggtgctgatgatcaaagttctcttgtcagcatggattataactgccgggtgcttcatcttcttcctcggtgagtgtatgttctttaagcacagacagtcgtccaacaaatggaacaacacatcactaatgctcactcgttaattacaggctttatcctatttgggatccttaagttctgcaggctctgctgtttctgctgtctccgcctggagtcgggcgacccccaagtagcaacatatcaaaacaatgtttaaaggtaagcattattaaatttccaattgaggtcttacgcatgactgattttgagatcatggcggggcacagccgggtgccacaggcacacgggagtcagtttcttatgatcaggctatttgggacagatggtgaggggaacgtacaggagcaaaggctctacgagctattatcgaacccccgatcagaactcggcctgtccacgggccccctcgtctccgagaacctgctgacagtcgcgctgcggggcggcaacgatgaccctaggcctcagcgccaagagagggcccgagagttggccctcgtcggcattctattgggcaacagcgagcaaggagaacgcctcggcacggagagcgccctaaatgccttgaggaacaagtatgtttatgcctacggaccagactggatggccagaccctcgacatggtccgcggaaatccagcagttcctgcgcctcctgggcacaaagtacgtgttgcgcgtggagcttggccgacagtttggctttgaggtcaccggagccaggccctccttccgacagttccaggccatcaaccacattgtcctctttgacaacgtcctgcgcaagtacgagccgggacaggcgtccgcgggcttccagagggctctgttggtccccggccccccgaccgtggacgtgaggccggatctgcgcaccgtcaacgagtgggtctttggcggtgtggccggcgggggcaggcagctggccgacgagctgatgatagtgtcgccgctgcgagacacctactcggggcacctggttcttcagcccactgaggctctggacgtttggagggcgtcgaatagggagagggaagcaacacacagtctggagcacggcttcgtccacacggcggggaccatccagcccaactgccctcagctgtttatgagacggcagcaccctggccttttccccttcgttagtgcaatagcatcgtcgcttggttggtactacccggccctcgcaggcccaagcccggatgtcagggaagacgccaggcgccagcaggccttccagaccaaggcggtggcggaatgtcacgccaggagcggggtgccggttgtggccggcttctaccggaccgtcgacaccactcttagaggaggagagggcctgacgcccaccatgtttaccggggagctgggagtcatcagacaccaggcgctggacaccgcgaggtacgagtacggccagtacctggtcatgctggggcctttccatccctggagcaacctcaccgcacctccgtgcccctacgccgagagcgcgttggcccaggctaccgtccagaccgctctggagctcttctccggcctctatccggcaccgtgcgtctcgagccatgcgcgcccacccggcccctgcgccctgatcgagcacctgcgatctctggttcagaaaggggggctgctgctgttcctgtcccacctgccggaggatgtgaagggggggataggggacctggggacggcgcaggccacgagccgggggataaagcagtacgtcgaaacctactttctcaatccaaactgctccaacgtgttcctgattgtgaggcagcggggggagaaaatcaacggccgcggcgtcctacaggccctgaagcgtgcctgtgaagtggcagggtgccaatactacgtgctgggctccacggtcccgctcggggggctaaacttcgtcaacgacctggcttcccccgtatccaccgcagagatgacggatgactttgcccccttcttcagcgtggaatttccctccatcccagagggcgaaatgaaatcccccgtccactcagacacggaagagcgcatggacgtctccccctctcacgagtcttcctggctctcgctgagctcctgcctcaccagcgttctgtcacaccccagcgtggggagcaaagagcacctggtcagacacacagacagggtctctgggggccgcgtggcccttcagcccggggtgggtcccctagacctgccacttgccgattacgcctttgtggcccacagtcaggtctgggtcaggccaggcggggcacccccgctgccctaccggacctgggatcgcatgaacgagaagctgctggtctcggcgaggcccaccgacgaggcccccaaggtctcgggtaccgtgattacattgggagaacaggggtacaaggtcacgctggatccgatagagggcaccaagctggccatggtcgaggcgctgatgaacgcggcctttgccccgctcgtgaatccggaagacgtcctgctcactctgaatctccacgcggacccgggccgcgtcgacagcaccgcggtcatggacttgatgacggcggccagcgactacgcgcgtgacctgggcgtgaagctgacgtacggctcggcctcctgccccgagacgggctcggcgggaaccggcttcatgacggtggtggtcacggcctcggcacgcggggaattctccgctcccctgatcacgccggtactgcagagatcagggagcctcctactctccgtgagacgcggggatggcaagatccagggcgggtcgctctttgagcagctcttcagcgacgtggccacggccccgcgggctccccaggccgacgccatgaagattatcttccggacggtccaggggctggtgaaaagcggcctcgtgctctcggggcacgacgtcagcgacggggggctggttacgtgtcttgtggagatggcactggcgggccagcggggggtgaccatctcggtgccggcagccgccgactacctcccccagctattcaccgagcacccgggcctggtttttgaggtcgaggagggaaacatgaccgaggtgatgcagaccctgcgggcccagaacatataccccgccgtcctgggccgggtgggcgaggcgggggcagatcagatgtttgaggtgcagcacggctcagagacggtgttccgccagtccctgagtctgctactgagcacctggtccacctttgccaccgagcagtacgagtttctgcgcccggggcgcctcaaccgatccatgcacgtgtccgactacggcgagaatgaagcgctggccatctcgccgttaacaggaaagaatctcggcctgcgccgcctggtgacagagccggaccctcgctgtcgggtggccgtgctctgtgccccggggaccaggggccacgagagcctcctggcggccttcactaacgcggggtgcctgtgtcagcgggtatatttccgcgacgtcagggagaccacgttctttgacaagtatgcggggctggccgttggaggcgtccacgcggccagggacgcccccctagccggccgtgccaccgtggctctgattgatcgctcacccgccctgcgagacgccctgctgaagttcctggccaggcccgagacgttttccctgggcctgggggagctaggggtgcaggtccttgccggcctcggggctgtggggtccacggacccaacgcccgagccgggggtagaaattaatgtccagagatcacccttgattctgacaacaaatgcctcgggattgtttgaatcacgctggctaaacttcagcgtgccacacgacaccaaatcggtcatgctgcgtggcatccggggctgcgtcctgccctgctggattcagggctcacatctgggactggaattcactaatcccgctatgccttatgttttacagaactctcaccaaatcgcctgccacttccatagtcatcgcactgacccctggcgccacgctgagcattatccacgaaatccctcggagcggagcaacatctcggggctctgctcacccgacggccgccacctggcactcctgtgtgacccgtcactttgcacagacttttggcaatgggagtacattccctccgcctttgccccctacacggggtgctctccatggactcttatgtttcaagcagcgcacctctggtcgctcaagcgcagccacccctcttcataaccagtcgcttcagtgacccaaaacagcgcagaccatagtctgggccagtcccagctcttaacttaaacttctgagttgagaggggggttcacgaaattagcacagaacacgtgtgctgtatttacaaacactacaataaactgtatagatatggtttgtgtgatttgtgttcctagaaaccccaatgagtggaaacctcagacacgcgacattaactttaaccacaagaggggtagtagttggagaaagtaaccaaatctccgggaagtctccaattgccttcggtttcctatgattatacaaatctagagttacacctcattacacaaatctcacgccctcaaaacccacagaaacccaagcggtcattctataagataacgagcgaaaataaggtgaggcctgcggacattgggccctggattagaggataggactcgttcgccccgggtaggggtctgagaaaagcgaaagggaaatgcgcaggcaagtaggtaacggttcgggaaatcacagcacggggctgaattcacggttcctgacgccaggtgggcaatatgtgatgaatcccccaaaatcacttctaggggaagcgaagtcttacaatcagcaccggggcggtggaaagccacgggaaggagtttctgttatcatcataaatggtagagaccctaggttgcgcggcggggtttctcacacaagtcccccacagggatccagaaataggtggacataaaaagctagggagtagaacaccgttcataccaccagatggcgggcgtgaggagaaaggagatattgtcaccccgcccagtctcagtgacgtaattcggacctcagggatattatgacactgagctagtgaacacccgggacctacgctgccctagcggttatgctggggaggagatgagtgtgacttaaatcacccgtcccgggtacaagtcccgggtggtgaagacggggtctggtgctgctaaaagtttggacagtccccggtttctgctctcttcggttcaaaccagctggtggtccgcctgttttgttgctgcttttgctttcagttttgaaaccctgttattatagcgcttgcacctggttctcccggtgctatttttggccaacaaggccgccgtgacgtagcataaccttgagttctttgatgttcaaaaccagtgaagcagttaggacattggctagcctagaggttacggacagaggggggtggtgtgcgggttcatccgtccgtgccgagtacaagtcccgggaaggggagaagagcggctcccgcctatgtgcaattgaagtcaggattctccaccctccctggcaacgccagccaatgtccgctcttttgtctgtccttctgctcccaacatccatactacacccgccaggaccccacaaggtccatcccatattataacaaagtcccctgtccaacactgctacgtcaaggggtacccgctagtgaaaacaggccagagaactcttggaattccatacgcttgacctccaatgtccctaagcaccgccacaagacccctcaaagaccaccaaacaaccagcaccggcttagttccagtcggtagtcatgcggtggcacccacccatgtctaagtggagggctagcaacaacagacgtagctgcaattctattattcaacggcagaagaagggcagcctgcgaaacttcacgccccccgagaaggttaagacccccaatatagtaggatagcatatgctatcaaaaggttacactaggatagcatatactaccctaacatatcaggataacatatgctacccaaagctaggatagtatacactacccacctaaattgggatagcatatgctacccaactaaattaggatagcatatgctacccaactaaattaggatagcatatgctaccgaaagctaggatagtatacactacccaactaaattaggaaagcatatgctaccgaaagctaggatagtatacactacccaactaaattagggtagcatatgctaccgaaagctaggatagtatacactacccaactaaattaggatagcatatgctacccacagctatgatagtatacactacccaactaaattaggatagcatatgctacgcaaagctaggatagcatatgctacccagatctattaggatagtatatgctacccgctattaagatagcatatgctatcccaactttaggacagcatatactacccagctgtactaggatagcatatgctaccccaatattaagatagtatatgctatccaatattaggatagcatatgctacctaactatatctggatagcatatgctacccgaacttatctggatagcatctgctatcctaattgttggtagcgtatgctatccaaacctatactaggacccccatcttcatattaatttcagacccccgaatctacttacccatgcccctactcatccccgtgaagcatctgcctacgagggggcatgcacccccgttctctctgggagccccactcccggggtcgctctctgtgctgttagggcccccgaccctccgtggcgcaagtactccctggggtgctattacttggttttgagggtaaaacccacaaacctcagttaccacagtggttaaaagggtcagggacgacattatgtatgcgaccccaatttccccagtgtaaacctcagtgtggtgagctgacacccctagcccgtgcccaccacttgttactcccagggcaaagcccccgtcctgcaccctgtactatctggggagccttttcgccggtagagtctcttgaacccccgggtatgtgagcgggtcagaaagactcgacggcccctgggtgtcctcgcccatgttctcgttacaatgagtgaatgaccacctggtacaccagcctagccccctctgtgacacgccttacttgggtcagcctacggggaccgtggatagcctatgctatctacagggtcagatgggaggtggtctctgacagtgaactcccacatcccatactcctggtcaggttggggagacatgaacaacaccaagattagctaaacatccaggttaaacagcctgcccccgcccccaggctcccagttttaagttagcaattcgtggccacattccacgccaggaatttcttgtctaaatggggctggggaagtttagcctgctgccaaagcacaaacacatcagtacaccactttaacactgttttgttcttgggtggtgctgaagtgctcacatttgtcaggccaaaggctatagctcacggcagaaaaccagactgacattctatcagctaaacggcagagaagcaaaatgcaggcgaagattcaggcccccgcccaggcgccccctcggacctcagccgccacccccgggctacaagggttcttcatggaggtgagctttcgaacctaggtcgtttccatggatactgttcatcgcccacgatatatagctgcaatctttgaggatgagattttagctgtgttggatagcacatactgccggccgggcagcatttgctatccggcgggtagtacatactacccaactagggtttgctggtgtgtttgaaaaacatctgcccccgaggaccccttggggtgtgaccccttggtaacagcctagataccattgtgacgcaccagcctacccgccccacgcgccttgggttcccgcggggccacacccgcctggcacaggcctatgatctgcagcttagggttgcaaaacatcccgaccttgagtcaggtctgagaggttgctgcgcttggtttcaatgcgcctgcccctcactgttggcaaatgcacacgtgcatttctgggggtgtaaggcacgcgtggatgccgcgttgggacgggcccttatactgccccccacatatgtcagctttgaagtggggcacggggtgccagttgccttggtcatcttctggggtagtatatgctatcacccgggcagtacttcctacccgggaacccgaaactttgtttcccagcagtgataaaaatagaacatacaggaattgccagggtgtgtcaccaagcctaaggcaagttatgctccataaattagctccccgcttcaggtacgcatgcacacggcatggggctgaggtcaggcctcactctgcaatgcctggtgattctgcaatgcctggtgatgctctatctggcacccgcgtgtaagggtgtgagcaattgtggcaacctcccccacatgctcagggacctgagagatgccttcagtcgggtgaagaccttcttccagatgaaggatcagctagataacattttgctcaaagagtccctgctggaggatttcaagggctacctgggatgtcaggccctgtctgaaatgatccagttttacctggaggaagtaatgccacaggctgaaaatcaggacccccacgccaaagagcacgtgaactctctgggcgagaatctaaagaccctaaggcttcgcttgcgcaggtgccacaggtttctcccgtgtgaaaacaagagcaaggctgtggagcaggtcaagaatgccttcagcaagctgcaggaaaaaggagtctacaaagccatgagtgaatttgacatttttattaactatatagaagcatacatgaccatgaaaattcgcagataattccataatctggaaaaggtgtccagattctggattccttgctccccaaaccccactcttcactttcatttacaataaaatgaaacttttcttcttggttgcttcttgtgttctgacccgcccaagtaccttcctgtggtctccccacgggagaaacaataggttctacagaagggcactgacgcagttttcgctagtggggtttacccggccagggctgttactgaaaatcatgttctctacccagcccacaagcacttagctctcattctatacggttaacgggaagagaagtggggagggcacacgccagctggccttaaaggccagcctgccaaagtccgccgcccattcactctatcctccactagaggccgcccatgggccatgagataggctccgaagccagccagcaggccatacggctctcaagccttgcatggggagacagcggggcggtgggcaatgcgccaccactgaaaggcccgcgaaattttggcatggccaacccaaagttagcccagcctgccacagtgcacctaaagcaggcggcgctgccggtgacgggggtcttctttgacggagggggtctcccgcgcgacctctgaaagcatttctaaccacccacccgcccacaggttgccccacccgcgatacccaccacgtttacttccaagattcccactatggtcctattttccccggcccaggtaaaaacacaggggccgaaattgcttcaaaggcgggcgccaccatgaagccaccgaaaccctgcccctgtgaatgtcacgggatgctaagtagtgctaaacaacccgtgggaaaaaatttatgcttcagtgcttctagtactattagttgctaccataaaaccctgaagcccgttggcgggagaaggcctaacgcttactaagggtggagcgaagccctcgggccaatgaaaaaactcgaggccattggcccctgggacatcgaaggtggagcaatccctaaaatgggggtaacttaaggcgtgtccacaaaaggggtcttatcgggtggagtttattgcgcctagtttgcgcaaacctatctaattctccctctacgtaaaatgtaggccttggccaatcagcggtccctaaggggggctgctaaggacccactacaaatactctgtgcactggttcaaatcttggatcagatggcatagaggcaaagaccaacgaccccaggatcctcattgcggggtcctcgccatgcggccaagtaggcccaactaacactacaaaaccttctcctactgttcatcatttaaccggcaaaaatcatctaaaccgtaagtctaacggcttcctggaacaaactcaggaggccgtgatgcacagtcggtattatgcatttttcttgggaatacaaattagatttattgtatatggtgttggactcttgtatgtccagattgctaatttatgtgattcttcttccctctaggagtgaagcagccagatttctgcacgtgagcatgtatcctaaataattattatctaataagtgaaataattagaaactcataaaagcattctccttccagtaactctaacgatgaactttatttgactgtgtgggttcctgtttggcggtgtctgtagacactagcttttcccatgtgagggctacttcccctttcatgagaatgtgaataatagaaatatccgggagactcgaaagttattttggttaagttcctcattttgtctttcatgaaatcttttagttgaaatttaagttgatcaagagggtctcaatcatgggagggctagtacacacgcttcttgctcttcacaggaacctggggtctagtctgggtgggatcaggctaacccaagtcgcctcaacccgggcttcaggcccgcctcagtcccctcgcccctgggcctcaggccctccgcgtccccgtcctccagagtcacgggctacaggccctgcctctccctctacagtcccagagtcccggtctggacacatgagccaggcgccggggcctttcaggccctcgggcccctctggactccggcttccccatcccagccgctccccggcctcctctcctccctgccttccctccctctctgtcctccccgcctctcccacgagcccatgtgtccccacaagcccagaacccggccctcgaggcctcacagcccaggccaggccaccttagacccggccaggccccataactaaagacccagtggccattctctctggtaacgagcagagaggaagtagaggcccgcggccattggggccagcaagcgagaccagcccagaggcccgaggttggagccggcgggcgccaccaggtcccaacacccggtcccctctagtggacagagacagggttagagccctccggcggccggacccgaggaggcgcctggggcgaggctgggtggctgggcaggccgggtctcgggtctgggggtctgtggtggtgagcctggtgcctctggaggccctggccccgcccggtggccttggggtagctctgggagtcagagagtcggcctaggcccggggagtggagggggatcgcccgggtctctgctggcagagtctgagcgatcctccgagactccgggccccaaggcggccgccctcagtcccccagacagaccccagggtcgccaggcggggtccggcctctcctggggcagcaggctcaacaccccggacctcccaggcccaagacccggcctgcccggcctgcccggcctcgctccgggcgcctcctcgggttcagccgctggctggctctgcccctctctctgtcctccagggggaaccaggggaccggcgccccagagcccctcgggtccgcctccaggcgtcctcctggtctccgctcccctctgagccccgttgaacccaaagaatgtcagaggggagccgccctcggggcccagggcccctagtccagaggacggaggagcacctcagggtgcctccccgggtcccaggccagccggagggaccccggcagcccgggtggccccagaggccaaagtcctcacccctctccagggcttcagagcccagaacgtcccgagaagggacccgagcagccccgtccccgcccgcccggccagagactctccctcgcggacaggggcctcccagggccctccagtccagacccagagacgcccaggaggccctggtccccagcagcctctcccctccctcctcccctcttccccctccaccccagtcccccccccccgctttcggcctctcgggtccaccaggccggccggagggaccccggcagcccgggagagccgccttacctctcccctggcctgtcctccccgcctccctccccggcccccctcagcttgtctccccaccgggtccatcaggccggccggagggaccccggcggcccggtgtgccagcgtcccccgcagccgcccagtctctgcctccaggcaagggcgcccgcctccccctcctctctccctccccgaggccccggctcctgagctcactgtcccaagtccagccccagaaccagcctcccaggcccgtccactcgccccgagtccagccccgtccaccctgtcccccgcaggccaggccctccgtcctccctggccactccccacccctccccggccaccccaggcccgtccaggggggactttaggtgaccctcgggcctggcttaccatagactcccatgtaagcctgcctcgagtaggtgccctctgagccccctttgccccctggcaggccagcccgacccccagccgcccccaaacctcgcccacacgtccaggggtcctcgaggaagaggcccagtccactcagccccgtgtccactgcccagttccccccaggagcccctaaaagtagggactcaggccatgcgcgcccagttggcgggcctgtcagacagccctgtctagggccgggagaggcagccccgaggcgggcacaggagcaggcagtctctggtagtgatttggaccccaaatctgacactttagagttctggaggactttaaaacttgaaaaatctaaacttttgaggcgatctgggcgccattttgtctccgcgtgcgcataatggcggccctggacccaggaaacgggtctatggttggctgcgccacgtctatctttagaggggaaaagaggaatcagtccctaaaagagggaagggggcttgatttttaaatttggtaaatgtcagggcccaggggggttcgcgttgccaggccaccttctcagtccagtgcgtttacgtaagccggacagcagccaattgccagttctagggagggggaccactgccccctggtataaagtggcccagcaactatttctggcagcatcagagcgccagaagcccacagagatgtaagacaggggtcttctacctcactctagccctccaggccctccgaggccccaggcctaacttctaactctgtccccttccttccctcgcccatcgccttgccctgcgcccggggccacctccaccgtcatcgccggctccgccatccaagcctaggggagacccaagcgaagccaccggaccttccaggcccgggcccccaggtctcgggccagaggtaagtggactttcatttattccgctcagcccaccactccaccacacccaggcacacactacacacagccacccatctcagggccccttcggagacctcctgagaaggcgcaggcacaacagcccaaccggcggagacccacaaggacccacacgcgtccgcaggagggtcttccttgaagaagaggacgggccggtaagcggtccaccagtaagggtaggtaacttcacctccagggcccacatcatgggagggctagtacacacgcttcttgctcttcacaggaacctggggtctagtctgggtgggatcaggctaacccaagtcgcctcaacccgggcttcaggcccgcctcagtcccctcgcccctgggcctcaggccctccgcgtccccgtcctccagagtcacgggctacaggccctgcctctccctctacagtcccagagtcccggtctggacacatgagccaggcgccggggcctttcaggccctcgggcccctctggactccggcttccccatcccagccgctccccggcctcctctcctccctgccttccctccctctctgtcctccccgcctctcccacgagcccatgtgtccccacaagcccagaacccggccctcgaggcctcacagcccaggccaggccaccttagacccggccaggccccataactaaagacccagtggccattctctctggtaacgagcagagaggaagtagaggcccgcggccattggggccagcaagcgagaccagcccagaggcccgaggttggagccggcgggcgccaccaggtcccaacacccggtcccctctagtggacagagacagggttagagccctccggcggccggacccgaggaggcgcctgggcgaggctgggtggctgggcaggccgggtctcgggtctgggggtctgtggtggtgagcctggtgcctctggaggccctggccccgcccggtggccttggggtagctctgggagtcagagagtcggcctaggcccggggagtggagggggatcgcccgggtctctgctggcagagtctgagcgatcctccgagactccgggccccaaggcggccgccctcagtcccccagacagaccccagggtcgccaggcggggtccggcctctcctggggcagcaggctcaacaccccggacctcccaggcccaagacccggcctgcccggcctgcccggcctcgctccgggcgcctcctcgggttcagccgctggctggctctgcccctctctctgtcctccagggggaaccaggggaccggcgccccagagcccctcgggtccgcctccaggcgtcctcctggtctccgctcccctctgagccccgttgaacccaaagaatgtcagaggggagccgccctcggggcccagggcccctagtccagaggacggaggagcacctcagggtgcctccccgggtcccaggccagccggagggaccccggcagcccgggtggccccagaggccaaagtcctcacccctctccagggcttcagagcccagaacgtcccgagaagggacccgagcagccccgtccccgcccgcccggccagagactctccctcgcggacaggggcctcccagggccctccagtccagacccagagacgcccaggaggccctggtccccagcagcctctcccctccctcctcccctcttccccctccaccccagtcccccccccccgctttcggcctctcgggtccaccaggccggccggagggaccccggcagcccgggagagccgccttacctctcccctggcctgtcctccccgcctccctccccggcccccctcagcttgtctccccaccgggtccatcaggccggccggagggaccccggcggcccggtgtgccagcgtcccccgcagccgcccagtctctgcctccaggcaagggcgcccgcctccccctcctctctccctccccgaggccccggctcctgagctcactgtcccaagtccagccccagaaccagcctcccaggcccgtccactcgccccgagtccagccccgtccaccctgtcccccgcaggccaggccctccgtcctccctggccactccccacccctccccggccaccccaggcccgtccaggggggactttaggtgaccctcgggcctggcttaccatagactcccatgtaagcctgcctcgagtaggtgccctctgagccccctttgccccctggcaggccagcccgacccccagccgcccccaaacctcgcccacacgtccaggggtcctcgaggaagaggcccagtccactcagccccgtgtccactgcccagttccccccaggagcccctaaaagtagggactcaggccatgcgcgcccagttggcgggcctgtcagacagccctgtctagggccgggagaggcagccccgaggcgggcacaggagcaggcagtctctggtagtgatttggaccccaaatctgacactttagagttctggaggactttaaaacttgaaaaatctaaacttttgaggcgatctgggcgccattttgtctccgcgtgcgcataatggcggccctggacccaggaaacgggtctatggttggctgcgccacgtctatctttagaggggaaaagaggaatcagtccctaaaagagggaagggggcttgatttttaaatttggtaaatgtcagggcccaggggggttcgcgttgccaggccaccttctcagtccagtgcgtttacgtaagccggacagcagccaattgccagttctagggagggggaccactgccccctggtataaagtggcccagcaactatttctggcagcatcagagcgccagaagcccacagagatgtaagacaggggtcttctacctcactctagccctccaggccctccgaggccccaggcctaacttctaactctgtccccttccttccctcgcccatcgccttgccctgcgcccggggccacctccaccgtcatcgccggctccgccatccaagcctaagggagacccaagcgaagccaccggaccttccaggcccgggcccccaggtctcgggccagaggtaagtggactttcatttattccgctcagcccaccactccaccacacccaggcacacactacacacagccacccatctcagggccccttcggagacctcctgagaaggcgcaggcacaacagcccaaccggcggagacccacaaggacccacacgcgtccgcaggagggtcttccttgaagaagaggacgggccggtaagcggtccaccagtaagggtaggtaacttcacctccagggcccacatcatgggagggctagtacacacgcttcttgctcttcacaggaacctggggtctagtctgggtgggatcaggctaacccaagtcgcctcaacccgggcttcaggcccgcctcagtcccctcgcccctgggcctcaggccctccgcgtccccgtcctccagagtcacgggctacaggccctgcctctccctctacagtcccagagtcccggtctggacacatgagccaggcgccggggcctttcaggccctcgggcccctctggactccggcttccccatcccagccgctccccggcctcctctcctccctgccttccctccctctctgtcctccccgcctctcccacgagcccatgtgtccccacaagcccagaacccggccctcgaggcctcacagcccaggccaggccaccttagacccggccaggccccataactaaagacccagtggccattctctctggtaacgagcagagaggaagtagaggcccgcggccattggggccagcaagcgagaccagcccagaggcccgaggttggagccggcgggcgccaccaggtcccaacacccggtcccctctagtggacagagacagggttagagccctccggcggccggacccgaggaggcgcctgggcgaggctgggtggctgggcaggccgggtctcgggtctgggggtctgtggtggtgagcctggtgcctctggaggccctggccccgcccggtggccttggggtagctctgggagtcagagagtcggcctaggcccggggagtggagggggatcgcccgggtctctgctggcagagtctgagcgatcctccgagactccgggccccaaggcggccgccctcagtcccccagacagaccccagggtcgccaggcggggtccggcctctcctggggcagcaggctcaacaccccggacctcccaggcccaagacccggcctgcccggcctgcccggcctcgctccgggcgcctcctcgggttcagccgctggctggctctgcccctctctctgtcctccagggggaaccaggggaccggcgccccagagcccctcgggtccgcctccaggcgtcctcctggtctccgctcccctctgagccccgttgaacccaaagaatgtcagaggggagccgccctcggggcccagggcccctagtccagaggacggaggagcacctcagggtgcctccccgggtcccaggccagccggagggaccccggcagcccgggtggccccagaggccaaagtcctcacccctctccagggcttcagagcccagaacgtcccgagaagggacccgagcagccccgtccccgcccgcccggccagagactctccctcgcggacaggggcctcccagggccctccagtccagacccagagacgcccaggaggccctggtccccagcagcctctcccctccctcctcccctcttccccctccaccccagtcccccccccccgctttcggcctctcgggtccaccaggccggccggagggaccccggcagcccgggagagccgccttacctctcccctggcctgtcctccccgcctccctccccggcccccctcagcttgtctccccaccgggtccatcaggccggccggagggaccccggcggcccggtgtgccagcgtcccccgcagccgcccagtctctgcctccaggcaagggcgcccgcctccccctcctctctccctccccgaggccccggctcctgagctcactgtcccaagtccagccccagaaccagcctcccaggcccgtccactcgccccgagtccagccccgtccaccctgtcccccgcaggccaggccctccgtcctccctggccactccccacccctccccggccaccccaggcccgtccaggggggactttaggtgaccctcgggcctggcttaccatagactcccatgtaagcctgcctcgagtaggtgccctctgagccccctttgccccctggcaggccagcccgacccccagccgcccccaaacctcgcccacacgtccaggggtcctcgaggaagaggcccagtccactcagccccgtgtccactgcccagttccccccaggagcccctaaaagtagggactcaggccatgcgcgcccagttggcgggcctgtcagacagccctgtctagggccgggagaggcagccccgaggcgggcacaggagcaggcagtctctggtagtgatttggaccccaaatctgacactttagagttctggaggactttaaaacttgaaaaatctaaacttttgaggcgatctgggcgccattttgtctccgcgtgcgcataatggcggccctggacccaggaaacgggtctatggttggctgcgccacgtctatctttagaggggaaaagaggaatcagtccctaaaagagggaagggggcttgatttttaaatttggtaaatgtcagggcccaggggggttcgcgttgccaggccaccttctcagtccagtgcgtttacgtaagccggacagcagccaattgccagttctagggagggggaccactgccccctggtataaagtggcccagcaactatttctggcagcatcagagcgccagaagcccacagagatgtaagacaggggtcttctacctcactctagccctccaggccctccgaggccccaggcctaacttctaactctgtccccttccttccctcgcccatcgccttgccctgcgcccggggccacctccaccgtcatcgccggctccgccatccaagcctaagggagacccaagcgaagccaccggaccttccaggcccgggcccccaggtctcgggccagaggtaagtggactttcatttattccgctcagcccaccactccaccacacccaggcacacactacacacagccacccatctcagggccccttcggagacctcctgagaaggcgcaggcacaacagcccaaccggcggagacccacaaggacccacacgcgtccgcaggagggtcttccttgaagaagaggacgggccggtaagcggtccaccagtaagggtaggtaacttcacctccagggcccacatcatgggagggctagtacacacgcttcttgctcttcacaggaacctggggtctagtctgggtgggatcaggctaacccaagtcgcctcaacccgggcttcaggcccgcctcagtcccctcgcccctgggcctcaggccctccgcgtccccgtcctccagagtcacgggctacaggccctgcctctccctctacagtcccagagtcccggtctggacacatgagccaggcgccggggcctttcaggccctcgggcccctctggactccggcttccccatcccagccgctccccggcctcctctcctccctgccttccctccctctctgtcctccccgcctctcccacgagcccatgtgtccccacaagcccagaacccggccctcgaggcctcacagcccaggccaggccaccttagacccggccaggccccataactaaagacccagtggccattctctctggtaacgagcagagaggaagtagaggcccgcggccattggggccagcaagcgagaccagcccagaggcccgaggttggagccggcgggcgccaccaggtcccaacacccggtcccctctagtggacagagacagggttagagccctccggcggccggacccgaggaggcgcctgggcgaggctgggtggctgggcaggccgggtctcgggtctgggggtctgtggtggtgagcctggtgcctctggaggccctggccccgcccggtggccttggggtagctctgggagtcagagagtcggcctaggcccggggagtggagggggatcgcccgggtctctgctggcagagtctgagcgatcctccgagactccgggccccaaggcggccgccctcagtcccccagacagaccccagggtcgccaggcggggtccggcctctcctggggcagcaggctcaacaccccggacctcccaggcccaagacccggcctgcccggcctgcccggcctcgctccgggcgcctcctcgggttcagccgctggctggctctgcccctctctctgtcctccagggggaaccaggggaccggcgccccagagcccctcgggtccgcctccaggcgtcctcctggtctccgctcccctctgagccccgttgaacccaaagaatgtcagaggggagccgccctcggggcccagggcccctagtccagaggacggaggagcacctcagggtgcctccccgggtcccaggccagccggagggaccccggcagcccgggtggccccagaggccaaagtcctcacccctctccagggcttcagagcccagaacgtcccgagaagggacccgagcagccccgtccccgcccgcccggccagagactctccctcgcggacaggggcctcccagggccctccagtccagacccagagacgcccaggaggccctggtccccagcagcctctcccctccctcctcccctcttccccctccaccccagtcccccccccccgctttcggcctctcgggtccaccaggccggccggagggaccccggcagcccgggagagccgccttacctctcccctggcctgtcctccccgcctccctccccggcccccctcagcttgtctccccaccgggtccatcaggccggccggagggaccccggcggcccggtgtgccagcgtcccccgcagccgcccagtctctgcctccaggcaagggcgcccgcctccccctcctctctccctccccgaggccccggctcctgagctcactgtcccaagtccagccccagaaccagcctcccaggcccgtccactcgccccgagtccagccccgtccaccctgtcccccgcaggccaggccctccgtcctccctggccactccccacccctccccggccaccccaggcccgtccaggggggactttaggtgaccctcgggcctggcttaccatagactcccatgtaagcctgcctcgagtaggtgccctctgagccccctttgccccctggcaggccagcccgacccccagccgcccccaaacctcgcccacacgtccaggggtcctcgaggaagaggcccagtccactcagccccgtgtccactgcccagttccccccaggagcccctaaaagtagggactcaggccatgcgcgcccagttggcgggcctgtcagacagccctgtctagggccgggagaggcagccccgaggcgggcacaggagcaggcagtctctggtagtgatttggaccccaaatctgacactttagagttctggaggactttaaaacttgaaaaatctaaacttttgaggcgatctgggcgccattttgtctccgcgtgcgcataatggcggccctggacccaggaaacgggtctatggttggctgcgccacgtctatctttagaggggaaaagaggaatcagtccctaaaagagggaagggggcttgatttttaaatttggtaaatgtcagggcccaggggggttcgcgttgccaggccaccttctcagtccagtgcgtttacgtaagccggacagcagccaattgccagttctagggagggggaccactgccccctggtataaagtggcccagcaactatttctggcagcatcagagcgccagaagcccacagagatgtaagacaggggtcttctacctcactctagccctccaggccctccgaggccccaggcctaacttctaactctgtccccttccttccctcgcccatcgccttgccctgcgcccggggccacctccaccgtcatcgccggctccgccatccaagcctaagggagacccaagcgaagccaccggaccttccaggcccgggcccccaggtctcgggccagaggtaagtggactttcatttattccgctcagcccaccactccaccacacccaggcacacactacacacagccacccatctcagggccccttcggagacctcctgagaaggcgcaggcacaacagcccaaccggcggagacccacaaggacccacacgcgtccgcaggagggtcttccttgaagaagaggacgggccggtaagcggtccaccagtaagggtaggtaacttcacctccagggcccacatcatgggagggctagtacacacgcttcttgctcttcacaggaacctggggtctagtctgggtgggatcaggctaacccaagtcgcctcaacccgggcttcaggcccgcctcagtcccctcgcccctgggcctcaggccctccgcgtccccgtcctccagagtcacgggctacaggccctgcctctccctctacagtcccagagtcccggtctggacacatgagccaggcgccggggcctttcaggccctcgggcccctctggactccggcttccccatcccagccgctccccggcctcctctcctccctgccttccctccctctctgtcctccccgcctctcccacgagcccatgtgtccccacaagcccagaacccggccctcgaggcctcacagcccaggccaggccaccttagacccggccaggccccataactaaagacccagtggccattctctctggtaacgagcagagaggaagtagaggcccgcggccattggggccagcaagcgagaccagcccagaggcccgaggttggagccggcgggcgccaccaggtcccaacacccggtcccctctagtggacagagacagggttagagccctccggcggccggacccgaggaggcgcctgggcgaggctgggtggctgggcaggccgggtctcgggtctgggggtctgtggtggtgagcctggtgcctctggaggccctggccccgcccggtggccttggggtagctctgggagtcagagagtcggcctaggcccggggagtggagggggatcgcccgggtctctgctggcagagtctgagcgatcctccgagactccgggccccaaggcggccgccctcagtcccccagacagaccccagggtcgccaggcggggtccggcctctcctggggcagcaggctcaacaccccggacctcccaggcccaagacccggcctgcccggcctgcccggcctcgctccgggcgcctcctcgggttcagccgctggctggctctgcccctctctctgtcctccagggggaaccaggggaccggcgccccagagcccctcgggtccgcctccaggcgtcctcctggtctccgctcccctctgagccccgttgaacccaaagaatgtcagaggggagccgccctcggggcccagggcccctagtccagaggacggaggagcacctcagggtgcctccccgggtcccaggccagccggagggaccccggcagcccgggtggccccagaggccaaagtcctcacccctctccagggcttcagagcccagaacgtcccgagaagggacccgagcagccccgtccccgcccgcccggccagagactctccctcgcggacaggggcctcccagggccctccagtccagacccagagacgcccaggaggccctggtccccagcagcctctcccctccctcctcccctcttccccctccaccccagtcccccccccccgctttcggcctctcgggtccaccaggccggccggagggaccccggcagcccgggagagccgccttacctctcccctggcctgtcctccccgcctccctccccggcccccctcagcttgtctccccaccgggtccatcaggccggccggagggaccccggcggcccggtgtgccagcgtcccccgcagccgcccagtctctgcctccaggcaagggcgcccgcctccccctcctctctccctccccgaggccccggctcctgagctcactgtcccaagtccagccccagaaccagcctcccaggcccgtccactcgccccgagtccagccccgtccaccctgtcccccgcaggccaggccctccgtcctccctggccactccccacccctccccggccaccccaggcccgtccaggggggactttaggtgaccctcgggcctggcttaccatagactcccatgtaagcctgcctcgagtaggtgccctctgagccccctttgccccctggcaggccagcccgacccccagccgcccccaaacctcgcccacacgtccaggggtcctcgaggaagaggcccagtccactcagccccgtgtccactgcccagttccccccaggagcccctaaaagtagggactcaggccatgcgcgcccagttggcgggcctgtcagacagccctgtctagggccgggagaggcagccccgaggcgggcacaggagcaggcagtctctggtagtgatttggaccccaaatctgacactttagagttctggaggactttaaaacttgaaaaatctaaacttttgaggcgatctgggcgccattttgtctccgcgtgcgcataatggcggccctggacccaggaaacgggtctatggttggctgcgccacgtctatctttagaggggaaaagaggaatcagtccctaaaagagggaagggggcttgatttttaaatttggtaaatgtcagggcccaggggggttcgcgttgccaggccaccttctcagtccagtgcgtttacgtaagccggacagcagccaattgccagttctagggagggggaccactgccccctggtataaagtggcccagcaactatttctggcagcatcagagcgccagaagcccacagagatgtaagacaggggtcttctacctcactctagccctccaggccctccgaggccccaggcctaacttctaactctgtccccttccttccctcgcccatcgccttgccctgcgcccggggccacctccaccgtcatcgccggctccgccatccaagcctaagggagacccaagcgaagccaccggaccttccaggcccgggcccccaggtctcgggccagaggtaagtggactttcatttattccgctcagcccaccactccaccacacccaggcacacactacacacagccacccatctcagggccccttcggagacctcctgagaaggcgcaggcacaacagcccaaccggcggagacccacaaggacccacacgcgtccgcaggagggtcttccttgaagaagaggacgggccggtaagcggtccaccagtaagggtaggtaacttcacctccagggcccacatcatgggagggctagtacacacgcttcttgctcttcacaggaacctggggtctagtctgggtgggatcaggctaacccaagtcgcctcaacccgggcttcaggcccgcctcagtcccctcgcccctgggcctcaggccctccgcgtccccgtcctccagagtcacgggctacaggccctgcctctccctctacagtcccagagtcccggtctggacacatgagccaggcgccggggcctttcaggccctcgggcccctctggactccggcttccccatcccagccgctccccggcctcctctcctccctgccttccctccctctctgtcctccccgcctctcccacgagcccatgtgtccccacaagcccagaacccggccctcgaggcctcacagcccaggccaggccaccttagacccggccaggccccataactaaagacccagtggccattctctctggtaacgagcagagaggaagtagaggcccgcggccattggggccagcaagcgagaccagcccagaggcccgaggttggagccggcgggcgccaccaggtcccaacacccggtcccctctagtggacagagacagggttagagccctccggcggccggacccgaggaggcgcctgggcgaggctgggtggctgggcaggccgggtctcgggtctgggggtctgtggtggtgagcctggtgcctctggaggccctggccccgcccggtggccttggggtagctctgggagtcagagagtcggcctaggcccggggagtggagggggatcgcccgggtctctgctggcagagtctgagcgatcctccgagactccgggccccaaggcggccgccctcagtcccccagacagaccccagggtcgccaggcggggtccggcctctcctggggcagcaggctcaacaccccggacctcccaggcccaagacccggcctgcccggcctgcccggcctcgctccgggcgcctcctcgggttcagccgctggctggctctgcccctctctctgtcctccagggggaaccaggggaccggcgccccagagcccctcgggtccgcctccaggcgtcctcctggtctccgctcccctctgagccccgttgaacccaaagaatgtcagaggggagccgccctcggggcccagggcccctagtccagaggacggaggagcacctcagggtgcctccccgggtcccaggccagccggagggaccccggcagcccgggtggccccagaggccaaagtcctcacccctctccagggcttcagagcccagaacgtcccgagaagggacccgagcagccccgtccccgcccgcccggccagagactctccctcgcggacaggggcctcccagggccctccagtccagacccagagacgcccaggaggccctggtccccagcagcctctcccctccctcctcccctcttccccctccaccccagtcccccccccccgctttcggcctctcgggtccaccaggccggccggagggaccccggcagcccgggagagccgccttacctctcccctggcctgtcctccccgcctccctccccggcccccctcagcttgtctccccaccgggtccatcaggccggccggagggaccccggcggcccggtgtgccagcgtcccccgcagccgcccagtctctgcctccaggcaagggcgcccgcctccccctcctctctccctccccgaggccccggctcctgagctcactgtcccaagtccagccccagaaccagcctcccaggcccgtccactcgccccgagtccagccccgtccaccctgtcccccgcaggccaggccctccgtcctccctggccactccccacccctccccggccaccccaggcccgtccaggggggactttaggtgaccctcgggcctggcttaccatagactcccatgtaagcctgcctcgagtaggtgccctctgagccccctttgccccctggcaggccagcccgacccccagccgcccccaaacctcgcccacacgtccaggggtcctcgaggaagaggcccagtccactcagccccgtgtccactgcccagttccccccaggagcccctggtatttctaaaactttcttacctttctccccttgctttgttatctaatgaacctgatcccccgctcacacctccattcacaaccaagccacaagggccacttatccagccaggagcaagggtaagtgtttttccattttgtagccctagccccacctcttaagtgctagtcatacctccaatcaccagccaccttccaatgtagtcttggagcgagtggctggcacgctctaggcaaagaacaaacccacaacaagtaaccaccacctgccgtcgaagggtctacatagaagaggaggaagagatctaagtcacaggcccagcccaggtgatttgtgacttccaatttattggcctcatttcagtacagctttcccagtcttagcatgtcagattctggtaatcagccttatgtgtaccaaggggcaacctagtagccattctatcaaattaacgagcaagggggctaaaattaagcaaggacagccagagggccgctgcgcccgccgggctggccggataccccggaaattggccgcaaaaagggctgtagagggtcacccttccctatctgcagcccttttctgcagtttccccttccagggctattcctagtgttgccacgcggtggcagcctatgacgaattggggtgccgtccctcccagggctaggtttgctgtagggccgccaggtggcgcaaactgagattaatgaccctgtattcctttccctccacccgcagtaactcagtgctggcgtgtgacatgctgtaaagttttgcctgaacctgtgttggggcaggtagataccaacagccttttaagcaccctcgcttgtcttttgctctttctgccgccaccatgcctacttactacgttgcggttcaatccggggggcagtatatcctacatgttgccactgatgaaagagggcaaccggtactttccctaacgtcaacgctgtccacccaggccctgtccgatcgacccttaattcaactcaaaattatcgtgggtgacgaaaatgagggcccgcaaactgtacccggggcacccccgccaccacctccgccaccccctcttccaccacctccgccaccccctcttccaccccctccaccaccccctgttcaacctcctccaccagaacgcagggacgtgggaacacaagaacctgacccccgcgataggaacccacttggtggcccaggggggagcgccgtttcgtgggcggttcgcatgctctgtatgtccaatttgattgtacgccaagcacacggtgtacgtggccttctagcgaccctaggtccacagagacgtaatcccacacctccccccatggcgtcacccatcacgttgtcaccaggttctgagaacgtgtcacccactccacccccatcaacaccaccgagggccgctccttcttacctacaactaccccaggaaccccagacacttaatcctggtaaccacacagcccaccctcagacaccatggatgcctcccccttctcaccacacacagtctccgtgggctccatgcaggcctcctcctcctcaacacatgcctcccccatcggctccatggaggccttctcctcctcgccccccgcagcctcattgggcttacaggcctcattatactcaacacacgcagcccccttggcccccatacagacctcctcctactaaacaccaggcacccccccgggtgccgtggagaccttctccagtgcaggcacccccccgggcaccgtgggggccttctcctactaaacagcaggcacctcccccggcgccgtgggggccttctcctactaaacagcaggcaccccccccggcgccgcccctaacatcaccagaacatcccgaccctcctcaatccacaccacatcccactcctcctaaacacatacctgtgcatccattgactcatgcacctgagggttctcctctccaacaagccccacagcagctcacccctagaaacctaccaccactcagttcacctcttaaagttccaactgtgcaattgcctcccctatgtacaacactgcccccacctgcgccatcggagccagctccgggaatagtccgcactgagttacagccccccgcgccaccagggcccgtatggtggccgccagttacccgttccaaggcctctaaacgccgaaagggaaaaagccgggctccctctagagcgcaaaagggcaagggcaaaggtaagggcaaggccaaagccagggccaggttaggccaagagtctaccccaccacaggcaggtccagtaccgtcacctcagcctactcccagtccgagtatgcctgcactaagtccagcaattcccctcgcctctaaccctgtccgggcgcccaatagctcaccaaccccgggcccatccgaagctctcccagcacgtagcaggttagccagggccgtggcaggccgctcagctcagccacccgagtccccagtacaggaaccgcagccctctgatactccaccaactcccatgctcttcccagaagactggtatcctcccaccatagaacccccagaccttgaagacattgatgaaaactgggatgacatttttaatgtatcagattctgagcattctgatgaaaaccaaaatgcaggaggtcccagtaagagacctcgcccctccaaccagtaaaagattcaatctgttcggttacccatgtatccaaaataaatttacctcctttctctgttaacagttaacccgtctgtcattctattagttaaacgggaaaagcaagaattagctcctcctccacatctcactgagagccccgccaaaattggcaactactcgcagcttgcaggtgggctgcatattcccgccagcttgcagttcacgcgcctctcacccacaaaacatcatctcaccccatgcacgtgcagccacaaatggcactccgttttccccacattcagcgggatgcttatccccgtcagcccagcctcactggtcaattcccccatgccctcatgtatggcattccttaacccagtacccaaagcccacattgcccccatccagccggctgcctatccccgcagcacctccttggctgcttccctcccagtctgtgtccaccagcaccatcccaaagttacacacaacacacgggactgttagttcagccagtttattgctacacagtatggagggccttgcaccccagcatttgacgccgctcgtttccaaatcttgtcgccagggtcagcacctgcaacagggaaacaaatttcagtgccgccgctacccccgggggatggttaaagcgggctaggcgtagggagtgagggtcatgtaggccgggcacagcagtatcttggctcggccctgcggctctggggcagccgggtggccgccggcgggttcgccgggccgctgccccgctccgggtggggggtggcccggctgggcaccgccgcgccgccgctcggtcctgggcctccgggtgctcctggtgctccggggcagccgggtggccgccggcgggtccgccgggccgctgccccgctccgggtggggggtggcccggctgggcaccgctgcgccgccgctcggtcctggggctccggggtccgggtgctccggggcagccgggtggccgccggtgggtccgccgggccgctgccccgctccgggtggggggtggcccgcctgggcaccgctgcgccgccgctcggtcctggggctccgggtgcacctgaaaggcaatgggggtcgggaagggaggggcgtggtccccgggaccccagcaccctcccggcctccctccctaggttgtgcccggcttgcctggccctggtgctcctccgggggtcgccgcgttcgctggtccggtttgggtgggcgtggtccgcgggttcggtgcacctggaaggcaacgggggtgggggagggaggggtgtggtccccgggaccccaggcccccacccggcttcctccccggggtgtgcccggcctacccccgttctggagctcctccggtccggtgggtgtggtccgctgggtccgctggtccgggtgggcgtggtccgatgggtccgcgggtttggtgcacctggaaggcaacgggggtgggggagggaggggtgtggtccccggaaccccaggcccccacccggcctcctccccggggtgtgcccggcctacccccgtcctggagctcctccggtccggtgggtgcggtccgctgggtccgctggtccgggtgggcgtggtccgctgggtccgctggtccggtgcacctggaaggcagggggggggtcggggcaggcgggcgtggtcccggggcctatgccggccgggggtcccgtggcacggggccgggggtcccggggggcagccgcgacccagcgcgccccgttcacgggggaggatcgcggccgggcctcccggagccccggcgagggtggggggtgcgcccccagccggaccctggtgccaggcagggacctcgcgccgcccgcttcatggggggggaggccgccgcaaggacgccgggccggctgggggttgtgcaccccccgagcgtctggacgacgctggcgaccccggccagcgcgccttcttttattcactttttgggggcgctgtgcaataccttaaggtttgctcaggagagggggcttcctattggttaattaaggtgtgtcattttagcccgttgggttacctgcaggtgtgtcaccaggtgggtggtacctggaggtcatgctattgggttaacgtgagagggaggagctaaggtccgcgagatttggggtaggcggagcttcaggagggtcccctccatagggttaaaccaggagggggaggttctgcgccccgttatacctagtgggtggagcctggaggtaggtatccatagggtttcattatcctggaggtatcctaagctccgcccctatataccaggtgggtggagctaggtaggattcagctaggttcctactggggtacccccctatcctacctcgaggtgcgccacccttcctcccttcgttataatggtagcataacctataggttattaacctagtggaggaatagggtactacagctgggtatatacctatgggtatatacctataggtatatagaaacctagaggaagggaaccctatagtgtaatccctcccccccctaccccccctcccttacggttgcctgagcccatcccccaccccagcaccccggggtgacgtggcacccctgcgtgcttttcgggtgcatcacctttgcacatttggtcagctgaccgatgctcgccacgcccgggtcatgacctgtcactcgccttgtcccgtggactctgtccccctgttagggtggctgcctttggcatgaatcactttgagccactaagcccccgttgctcgccccgcctgcctcaccgtggcacactaatcccctgctaatccatgtgccccgcctttaggaaacaccgcgtcccagggagggaagagggctctgggagatatcctaggtttggacggaaatatgagtcagagcacggccggctccttattaacctgatcagccccggggttgcctgtcttagcactaactccgggcctgaagaggttgacaagaagggtcgaagtctcgccggtgtgtttggagggctgtttggtgcaactgaagctgctctcggttggtaaccgcttgacattaaagggttggggattaattttagccccttaagctgctatctgaaaacggggtgtgcatacaccagccaccagcctggggttcggccgcgcgtgttagttgcttgttgatgccttttgccatcttccccctcctagaaattaggcgcccgtgcctcccctcagagccattcccctgggtctgtgggcagctgccttagccccccactccccaacctattggggtaggtaattcttgtgtggtgactttgggagcgtgatgtaattgttcccaccctcaattttcgctggccttcttccctcgttaatttgatagcatggctgttgggtcttactgtgggtctgtttgtgcaggtggcttcatgtggtgctaactttcatcttttggttttctagttctcgcctaattacacttgtgccagattttgtagataaagatggccttttcaacaagagatttactgttagccctgtgtatgcgggacagtcacgttcatggaaacgagggtctgcatcctgtgttggagttagcatcaagagaatcaccctccagcctctctgcgggggacactctggttctacgtgtccatgtactgcttgagcaaataattgaacaaaatgcgggtgcctttttagaagccttggacagatttttatcaaacaccgaagagtgggatgtggattttaccagagtgtttcaagaaatcttccaccgtggaaatcccactctgggacgagcgttggcctggctggcctggtgcatgcatgcctgcaggacgttgtgttgtaacaggaatactccctactatgttgtggacctgtcggttcgtgggatgttagaagccagcgaaggccttgatgcttggattcaccagcaaggcggctggccttctttactgaggggcaaccgttcaaacaccggaagccccatattgactgcatttttagctggactaactttaactttcgtagttgtgtgtagctatctctttatctctggaagaagacgctgatttatatgcctgtattcagcactttacataagcttgggtcattgggcctgttgaaaagtaaaccggcaggatagatgagtgggtgagggtgctacagtgcccacaccttgtaactgaacaatacagtgggtttagtcttaaagccaaccacgggggcctgaagctttagaaaccaatgacccgggcccccacttttaaattctgccacagaagatagctgatactcgatgttatcttttgcgggggaatttccaagtgttggccatacacaccaggggcctattactgagacatgcccgtggtgttctgatggacagcggggaagtgcacttgtgcttatgtaatcggtgcttcgccctctccatcataataacacgaggatgagaggctaaggataactaaggtgagtgtaagctgtggttgggaagacgtgtatgtttttccagtgtgccattaaggggtggggttgaggcctgccttggttaatgcatggtgttttacatgcactctaaggtgtggggtttagatgattgggggcggcgtatcttttgatttggtaaaagttagcccctccccatcatcttgacaccacatttacctatcatcttccagttatgcttggtgttactgttttgttcaataaacacagattttataatacaattgttgtgttttctgcatggggatggtgggtagctgcctcgagtgtgccgtgccggtcactgctgcagttatgagtccttgctttgaatgtggcccgggtgggcaatggctggcccacgtgaatgctctttctcatatcccaaaaagctccccggccatgcaccacatcaaagcccagccaagtccccatttatgacttagcaagtggggctttgcaggacccaaattttgcaagctcacccctccagtccactgataatctcacactggtatgacttagccagtgggtcttagcaggccccaagccccgcgaaccctccctgtttcaccccgttattccaatagaatgacagattggctttcacaaccagtcaatccacccctggctacccaacaaacacaggacaggtgttaagggtccgagattactgtttattttctaaaattagctgtgtgtagttgatgatctgcagtcttagttcgtccccgcactctagatccttaatggcctcgaagatggcgtcctcatcgacgttaactgccttctcgtggacagcgtccctggggctgacttttacacagaaggagtcccctatcagatccacggccagttcgtatgcggggccaatgttttcacagaggagtttcaggcaggtctccgggatgtgtagggaggtgccttggagcagaagatgcatgttcaggcgtcctctcccggtggtgctgaatatggggcacacagtgccacagaagtggccggcgaccagataggcatagagcaggccgggctgggagagtcctgccttcacggcgctgggggagctgctaagcagggacacatagaaggtcttattggggatgatcttgtggacgttattgaagaaggagagctgggtggagctgaactcctgaggcacgtgaacctgggatttgttgatgcagacctcacagtgggagcccagagtcaggctgtggccaaagggggagagtcgaaggcagcggcccggagacagtgtacagagagacagcggtaggaacacggcctctgacacgtggatgggggtgttcatctcacgcagaaaatctttgcccaactcaaagttggcagagatccccctgaagaattcacgcagcgtaaaactggacccgtctcggcgccgtgtgtgcctggaacatctggagcgggccacactgccggccctttccatctttcccagccccagatacgttttgccgctccgtgccaccgatctcacgctccgacccgaggccttcgaggccacgtagtctctgtggtgcggatgcatggggtggtggcatctgggattagcgggcctctgctggcacacatcccctgcctccggggtgacgggtgccatccgggctacttttcacacgtatacctgggatgtctgataagactggaccaggtttaggccgtggagacgcaggaccccgtccagttccttagtgacatccacgagtattgttttgcccactctggccacttgtgtggatttaaatatgtacacaagcgtaattaacgcgtcgcataccccctgttctaggttctgcccggccgcgagagctgccttaaaggcctggaagctgggtgggtaaatctgaccaaagagcacgctcggatcggtgatggcgtggttgatggcacatagggggtcgcagaacaagtgcttgtggaagtcctgcggggtgcacatctgcagccaggccctgagcctggggcagctcacgtccagcagcgcgttctgggttttgatgaggaacacaatcctgtccaggattttgatgttgtttccaaaggagaccaggataaggctcttgaagcggtcgagggtggccttggcatccgggtgggcccccagactctcgcagagggggcagatggtccgtgagccgttcttgtgtcgcaacccaaacatgggggccagcaggcaggggccctgtgaatggtcgccggcctccggtctggggaaggccagggccatttccgcgagcgcatccccgctgtattccgcgtttaaaacgatagcatggtggccgggtcctcccagcaggtccgtgtctctccaggtgcccatcagcaggccgcagtagtcggccttggacgcgacaccctgggaatttcctcccttctcggccctgaggaacgggtgtgggccccggactcgagacggcaggtggcagcagaggcctgtgtactgggctcggctggcctccccgggaatctcctgcgattctttggatgttctgggtcccgggagggtgagaaagggggtggttctgggccacatggacttgaagcagaagttggccggggcctggcccgtgaggatggatttaaggaactcgagtttgtagtagcccaggttggcgttgcgaatcatgtctgaggaggacacggggagaaagcaccgggagatgtaaaagtggagctggatgtcaatggccaggatcctggagggcatgaagagggactccagcccccccgccatggggaagtatttcatcaggatgtgtaaaaagtccacgcccgtgatgaggctggagatccaggctctgggggcgttgagacagtagtagcataacagggcatagtcctcgaagaaggcaacgggggcctcggagtgattgaccagggtgtccagcagatcgcagacgcggcaggtgctggctggagatagggactcgtaggtgtggactagcggggggtaggctaccccttcctccgcgttggcggggaggtaggagtgggtcttgagcatccttgccgcctccggctggcttttgagattgtccacggtccaggggacaaactcctccatctctggagttccgccagcaccccgggtccctgctattaagtcactcctagtgagtgggtggcccggcagcctttgaccggtcagacggggcaactcgctcgcgcttgtgacgctgtgagccatggcacatgtgggcggcggggtggggtctcgtgggtttttaataccggagccggcgcctgtttctgggacaccgtctggaggggggctggccatacctggagttgacggcgacccgggtggcagtcttttgagtttctcgcctatgatccgcccagatctcgcgcccctgattgcaccgtccgcctacgcgaggctgaacctcctgaatcactgcctctttctggaggcgtttaaggaagagaatagcggtcggggcctcggtggggttcaacgagaggtcttatttttgaaagggacactataaaacaggtcattgcccggattgtggctgctgacagcaccctagacctagttaatcatggtgagcccggaagagaggctcctagacgagctcaataacgtgattgtgtcctttctgtgtgactcggggtctctggaggtggagagatgctcgggggcccacgtgttttcccgggggagctctcagcccctctgcacagtgaagctgcgccacggacagatctaccacctggagtttgtctacaagttcctggccttcaagctgaaaaactgcaactacccctcctctccggtgtttgtgatctccaacaacggcctggccaccaccctgaggtgctttctacacgagccctcgggtctcaggtccgggcaggcgggcccgtgcttggggctggccacggacgtggacctgcccaagaactccatcatcctgctgggacaggacgacttcattaagtttaagagtcccctggtgttcccggccgagcttgaccttttgaagtccatggtggtctgtagggcctacatcacggagcaccggacgacgatgcagtttttggtgtttcaggccgccaacgcccagaaggcgtcgcgtgtcatggacatgattagtgatatgtctcagcagctggccctgtctggtcagggcgaagagccgggcgtcagagccgtgggaggagggcctaggacagcttccgagggctcgctcggacgggggcgtggcgggtgggccgcgggcgatttttcagacgccgagactgacgacgataggagtcgcgctccctggagggacgaggactggtcggggtcggaggcggcctcgtggaagaaggaactcgtgaggcgccccatccgcgggcactgggcgcgcgtggccaggcgaaagcgctctcgccattcccgggcggagcacgcgcccgcggaggacccggcgacggagacggcggaggcatggcgtccctccctgtttcgcgccccaacctatctggcccagggcctggcggtggtggccgtggctctgtctctgctgtttctgaggtgggcctgatgtccgcctggggggcacccgagttttctaggctcctagatctggggctggcctgtctggacgtgagcttcgtgcagatgcgggagtttgtggtctggggcaggcttccggccccagaggaggccgccgcttccctctccggctctctctttagaagccactcggccgcctactggctggccgaggcggagaggcccggggggctggtccgctgggcccgggcgcagaccagccccacatccctgaccctcgcgcctcacctgggcccgtccctgctggctctttcagcgctcaccggcggggggtgcggcgctgtggccttttgcaacgccttcttcctcacctactttttgctggtgcgctctgttttccctgcgttttctgagagaatagccgcctggatttgcacccggtcccctttctgtgaaaacacccgggccgtggccaggggctaccggggcctcgtgaagaggtttctggcgttcgtgtttcagcgcagtagctgcgacccccccttgctgaggcagaacccgaggcccgtggagcgctactttgccatcaagaattatgttcccggcctcgacccgcacagctgcgtgacgttgccgagctactccctctgggcccgggctcacgccggtgagctcgaccctagggagattcgcgacagggtgaccccctctgccgccgcccctccctgtgtggccgatcacgcttcggccctgctggccacactccagaagaaggcctcggacaccccctgcgggaatccaatccagtggatgtggtaccgcctgttggtgaactcgtgtctgcgaggcgcccgttgtctcctgcccgtccccgccgtccccgaatgcgggaggaggatgggtggtggcggggaggaggagcgggagccggagtgccctgcgaggttgctgactggcccgggaggtcagtgtctgagccgggacgtcttcgtggcggtcgtgagccgcaacgtcctatcgtgtctgctgaacgtgcccgccgtgggccccctggcctacaagtgttttaggtcccgctccccctcccgcccggtctgcggcccggatcaccctcccctggccgtgttctgcctggactgcggctactgcctgaactttgggaagcagacggggggtgggggggcgctgaattctttcaagcccactctccagttttatccccgcgaccagaaggagaagcacgtgctgacgtgccacgccagcggccgcgtgtactgctccaactgcggctcgcccgcggtgggctgcctgaggctggcagagccccccagcgcccgctcgggctggcagccgcggatccgggcggtgctgccgcacaacgcggcctacgagttggatcgtggctcgcgcctcctggacgccattgtcccctgcctcggcccctcccgcacctgcatgaggccggtggtcttgcggggggtgacggtcaggcagctcctgtatttaactttgcgggccgacgctagggccgtctgtcccggctgtcagcagcggcgccaagctccggaggactcccgcgacgaggcccatcctctctcctcctgtttagaggtagaattgccgccgggtgagaggtgcgagggctgccgtctctatcacgcgcggtatggcacgccggctgcccaagcccaccctccaggggaggctggaggcggattttcaggacagccccctgcttcctaaatttcaagagctgcagcagggcaatctccccaacgacgttttccgggaggctcagaggagctacctggcctttctgacggcccagtcctgctacgaggagtacgtgcagcggacctttggggtgcctcggcgccagcgcggcctggacaagaggcagcgggccggcggtgccgcccaggttagtcactctcacccggccggcgcggcctcggccgcccagggccagcaggcgcagcccgtggcagggggtggcttagcgggggcagcagcggctgcctctgccccggctgcgacccccgccgtctcctcgtccatcagtagcctgcgggccgccacatctggagcggcagcctcctcggcggccgttgacaccggctccggcggcggggcccagccccaagacacctccacgaggggcgcgcgcaagaagcagtaggagacgcggcacgcggtgtatgcactttattaataaacaattacagatacaaaaacttgagtctctccaggtctgtgatgagccggtgggtggaacgctccagtttgtggcgaagctgggtcacgaagcgagtcaggacgcggcgagcctgggcgagcgtgaggctataacgcatgtcctggtcgggggttgcggcggacagagaggagtgggccgcagaggggaccaccaggtctccgggcgtcgacccccctgacaccacctccctaataatgtcttcaatagacaggaactgggtgacaactgagggagtattccacaataattctggttggtcgctagatggcgtgggagaggctttatcacgcgaaaacgaaagtgtttgaaaaggcgcgggatagtgtatgctacctgatggcgggtaatatatgctatcctcatatttttgggatttgcgcggcggggagggggcggccgagggccccgcttttttagcgatgtatgttggggcgcctgcatgccgggactggggtggttccacctcgggccgcgcgctcggcgccgaacccggatgcattggagtcaatgggaatgcaggagtttgttgctgtttgcttttgacagggagaggcacgcgcggctgcactgcagccgggggcggtgccttggtgaccgtctcctctggccgggcggcggtgtccattgcccctgcgggccccggggtgtgcccaccgaccaccgccaaaccggtagtgggggctggggccgtggcagccgggggcttctgcacaggtggctgatggggacgaggtgccagacccccgcccggggggcgtgtggggccgggcacggcgccttcctcgaggggctcccggggtctggcacccgtgccatagcctcccgtggaatcccacgcaggaagcgtggcctgtgccgtgctcgcgaccccgggcgtccggggtttcaggctgacgggcggaggacggtggggggctgcggtggactgaacctttgcgtgttcctcctggcccccgggccgctgcctttcccgttgcatgatagaatggccatggggtggagctggagaggcgtctcccaagcccgggcgcctgggtcccaaaacggacgatgatgccggtgtggcggcggtggccgtggcccccgctggccggcctgatgcggtggccgcgggcggctgtggttgtcggggcccggcgggcggggaacccaggccggggatctggatggtgtgtttctgaggccccgctgcggctggtgacggaggtgggtgtaggttatagatggcgggtccccgctgagaagcccctgggctcgcgatggtaccttgagcggccggcgctgggacaatctcttgaacctctcgtggccaggggcgcggggaggccccgggggactcgaccgcggtggccggacccttctctccagctcggtgaggccccaccgtccctcgagagtgagggagagaggccggggccggctccctaggggcggctgaggtgcctcgtggggagagggagggtgccggccattctccggctcggccggtgaccgccgggagccttgggctttgcgggctgggtgggacggaggcctcggaatcggcggcggcgctcccctcctcatcttcctccgtctcccctcccggactctggaaccggacggaggctggggtgttgtcggccgattggaacccgtccccgctcaccagcccatgccccaccctcggtggaaagccggagaacagctcctggacgttgcggggattcgggggaggccggtacccaacgggaggagggtcttcgtgttcgtagaaggaggggttgagcacgtcggtgggccagcgcgaggccccggccgggttgaagtggaactgtaggtcaggcagattgtgccggtatgtgaagcacttccagacgatgttttttctgttggccaagacggccacggtgggggcctgggcctccttgggcttggccgccctggcctccgcgaggagctcgcgcagccaggcggcctgcctcgcgaagacggacatctcgggctcaaaggactcggagtagccttccaggtccttcataaaatccagggagaccgtctccgccagggagcggaagggctctgagcgcccgtcgcagtagagcagcggggccacgaccctgacctggcccagagtcttcacgttaaacagatactgggaggagacgaagagagtcaggggacggccgaggaaggccgccgccacggccgcctcgaagacggagacggggatggcgtcccccgagccccttttgggaccggtgatgggggttccgtagggcatgagatttctcagcgcccggccggtgaccgtgccgtaggtggacggggtctctctggggacctcgacgctctctgcccggggcagctcctcgccccgcgtgtaggcccgcctgacaaagtcgcgcacgtagtcttggaaggcgacggggccctccaggggacgcagcgagtgccatagctgctgcagggcctcgggggcgaagcaccgtcgcgcgagcagcagcaggcatgcccgggccctggccgtgctctgattgtggaccagccccaggaactccgggtggggccagagagcggcccgagtctctatctcctcccaggcttcctggaggaaaatgaagccggagggagggccggtgaagcggcggtgcgcgtccccgacgttttgagccccggtggcggcggtcgagctcgagcccctcttcctctcggccgggcttctgaccagccggtaggtgaggaccgcgtccgacagcagctcccccaggcccccgtccctcgccagggccgagaccaggccgagtcccgcgtagaacgagggggcccccaggaaggcggcggcgtaggccggatgcgtgccgaccagcagcgtcattatctcccgttgttccaagagaatgacttcccggtctgtggccggtgtagggaagggggggtgattcctggaggcgatcagggtggcgtgcgccagcagcgtcatggccacgatggggttgtcggccaggtcttccatcagggccttgggggccgagacgtactccctgagcagttccccggcgttggcctccacgtcggaccaggagtcccagtaagagtcggccgcggcggctccgaggcgggcggaggccacgctggccagggtcctcgcctcccgaccccgggcggatggctggccggccggcgcgagggcaaaggcctctgggttctccagcacggtgggcatgacgtactccagaaagttgtggtagacggggatgtagttgaggggctcctgggtgtccgacgagacgtaggccgggttgagggggtcgcacggggactcggtctccagccacagggtgcccgcgtatttcgccggcccggccgccgccaggaattgcgcccgtcggaccggcagcggcccggtcggactcgcgggcgccctgggtgcgagaaggggccggtcgccggtgtcggtgagggggcgcctgacgaagacggtgggctgcgccgtctcgtagaaggccaggaagctctgcttggccgaggcgtagcgcacgagcctgtccctgaggagggcatactggggagagcccgccgccgccccgagcgccaggcccctggccgcctccgccacgatcttgagctggcgcgggtcgacgtggcccctggcccgggtgacgaggtcctgcagaacgtcctgcagccgggactcctcccgggcctcctggatgatggcctccagccgggccaggcgccgtttccagtctgcgacggtctccttgccacccgcgacccgcctggggtcgaccgtggccagggccagcctcagctcctccatgccgtccatggagttctgggccatgctctcgagctccaggagcctcttcagctcgccgatctggtcttcggccgccgcggccagttccagccaggttgcggggcccccggccagcgccagagctccctcggcgagacccctgacggccccgcagacgtcccggatccactcggcggccgccagggatcgccgatagggccacgagccgtctctggccgcggcccggcccagcgcggcggccagggtctcgtggatggaggacttgggcgtggaagaggtggggagcagcgcccggagctcgtcgtccggggcctcctcgcgcgccctggcgagtacggcctccagggccgtctgaaagccccgtctagcgcccaccgccgtctcatgggctcgggctgccagagcgcggctctcgaggaccctctcctccagcgcccggacctgggccgcctcggcctcggtcagcagctccgagaagaagtcccccgtggcctggaggagatcgtcccgctccctcttggtcaggagctgcgatttctttggccagagcgtggggtcagaggtggctggtggcgggaggcttggcgggggaggcacggcgggccggctcgggtcgtccagcgccccgggcagcgtgcggtccacgtactcgctcacggcggccgacagcgcggcctcggccgcccgtctcttctcttggagttcggccagcccccgctcgtacccctcgtagggggcctccagatccgtgttgaccgtcgccgacttggcatccggggactggagggcctggcgcgcctgggtgagcgccgcgcggagggcccccgccgccgtcctggcgcgaaagagggccccggcctcctccgcccctcgccctccaccgtcggaggtgctggcggtggtgggccaggcgtccagtctggccagcgggccggcctcgagttccgcgaaccacccgggctccgcggcctccattctctcgaccccgccgccgtccccgagcaacgctcgcagtctctcccttttcacccgcgttatttccgatagcatggcggccaggatccgcgtgagggtctccagcacctccgcgttcctggccgccgcatccgccttcgccgttgccgcctcgaccttggaatcatccggggcctcgggggtcagggaggccagaagggtgttccagtcgtcgggtgccgtgggggcgggctccgggcgggtgcccagcgccttctgaatcctggcccatgcttcgccggcctccctcgcccgccgctccgtccgctctctggcctcccgctcgcgcagcagctccttctccttgcgcccggcctccagcagccgccgcagcttcttctcgtactgccgcttcagctctttgttgggggcggtgtccagaaaggccttcagctgctcctcggaggcgggcgcgaagccgtcggcctccaggcgccaggcctgcgcctctttctgtctcagctgatcgttgttgtcattcttcaatttttgcaggtaactcagaaagcgtttcttgagcttcccggggatgagcgtctgggagagctgattctgcagaccggctaggagccgcagggcgttctccggggcgcggcccgagaccgtcgcgtcgtagacagccaggagacccgggagcagattcaccgcagcggccgtctccctcaaggtgctgtgagtagcgaaattctgcaaagccaccaggcgcacgggttccagggtcagcctgttgcccgtctcgaaggtgtgcagggcctcggagaccatggggtccaggatacggtcgatgccctcctgcacctccgggtccagggccggtagccccctgtagaggtggtctatgctctcctcgaaggaggagatgtagttgtcgacggtgtagaaggtgatggattttaggatgttcatcaggtacttcttggagcgcacgatctgctggatggtgtcgcgcaggcggatgtaggtggggttcttggcggcccccacgatggacccggcgttggcgatgtacttctccatgaccgggatggtgagggccgcggtgtcggccaggggcggcgtggcatctggatcggcgggtgatccgggggctcctctgggggcggaggccacgccggccaggcccagcatggcctcgatgtcctccaggatgacgcggaggcgcttctcgttctctttggtggcctcgagctctttctgcttttcggcgaccgtctcgaactcgggcaggggggctatggcggcgtcgtcctcctccaccctctccaggggccacggaatgccggtcatgtcgcctagggcggtgcccaggtagagcagctcgcggtagtcccgctcgatgctcgcgtgccggatgttgaggtccgacagggaggcaatgatctggcacgtgtgccgcaggcgggtcaggaggtcgtcaaagtccatcaccgtcgggaggctggggtcctccaggtaggagaggtagtcggaggccgccgaggccactcggtcccggacgtccgccgtgtacttgcgcacgtgcaaggcccgcggttttggaccggccgccacattgtgcacgaaggggggcaccaccgaagcagaaggggccccgtactctatcgccgttagcagggccaggaagcggacgtcctcttcgtccacggcggccccgcttgtccggccacgcggaccggcgacctccgccagggacatgaggcgccgccagcgctcgtcgtccaggaccaggggggcgtttgcacttgcggcgggctcgggtaggcccggggggtgagtggtcaaccccttcctcttccatccgttcttccttgacggactctcgtccttcgtcggggccaaggggcgcaggagatctgtcaggcgccgccgggccttgtccaggtcaaacttgccatccgcggcctcggccagcctggggatcttggccagcagtcccgccgccgcatccacgcggctcgcgtcctcggccgccagggacggcaggcaggccgcaagcttcgcggccagatgcatgtggaattcgctcgctcttcccttgttttctgatagcatggcggcgaggttttggacgttgagctcagaggtcaggagccgctccaggtcccgggtgggcacctgcaggtttccagaccagtcctttaggaactccagcagattcagggcggctgagcgtgaccggctcctgatgagaccctgctccaagaccaccgtcaccagcagatggtctatcgtgctcagggcgtccctccggcacttccctgttagaggccgggtcatagacgagggcccggtcggggtacgtgtgtccggcaatcttttgaatgttgcgctcgagcgggctaaagtccatctcgggcgtggccatgtcaaaggtggtttgatagtagcggctcctggcggtgtccaggctggtgggggatgagaggcgttggccattgtcctcctccgtgaggtccgaatccggccgttggagaacacatcatcctcctcctcctcgctggtggggatatcggaggcaaagtccatgtcttcttcagattctgacatggataggaaaggctcctctacgtcagacaggtagcgtacgaggccggatcccacggaggcatcttctcccgatgactgccgctggacagaaggcagtggggatgttgaggaaggaatttgttctggctcggaggtctccactggtggaggctgaggagtcgctgtagctgtcggagtgggctgtggagccggggaggctgctcgaggaagctgtggagcatgggaggttgcttgggggggctgaggagttggagtagccgcttgagggggctgaggagttggagtagccgcttgagggggctgaggagttggagtagccgcttgagggggctgaggagttggagtagccgcttgagggggctgaggagttggagtagccgcttgagggggctgaggagttggagtagccgcttgagggggctgaggagttggagtgcgcgggactagaggtgtggagatgggcgggactagaggtgtggagatgggcgggggctgcggcgtcgggagttgggaagttgtggggtctggcgggggtttgggagttgggctgtgagggatggggattactggagatgggtggggaggcggcgcggccgaaggggggactgaggccggttgcgtcgggacagccggctgaggttgagggacggtgactaccggagcagggaggcgagtaagaactgaaggggggcgacggggcgggggctgatcgtcctccgtgtcggtggtggaggacatggggaggcggcgccccgaggtgggcttcttgtgcgggctgtccttaccaggcccctttttccttttaggggtctgaggcgccaagccacccgttccccggggcggcgtggaggggggctcggggcctggcccgagcccggggatggggatgaagaggggagggctggaggctgcgtcggcgggggcctccgaggcaccgacgggcggccccggtgcggtctgcgcgccaactggcgccgacggggaggtctccggcatgcgggccgggcgcgtttcggacgagagagagcgagagggcagcggcctgaggttatacggtattatttgccgttgaaacggtagcatgacgggagggctgcggtcgggggccggcgaggccatctgttccccgtcgctggacaccagctcgggccccaccaggtcgaagccgctgccgttggcctcgtaaaagtcgtacacgccgtagtgctccagcataaagacccgagggtcctccatctcgaaggcctcgggcagaaagtagaggtggacgcacgtgtactgggccccgggcgcccccacgtactggaggatgtcatgcgcgtaggtgctgaccctgacgtgggccggggtgcccggggcggcgtccttttggcagtgggggtcgaagaggtagaaggagccgtccgtctcgatgatgatggcccccgcgtagatgtcgcatatgtagaggatgaactgggccaccccgttgtagccgccgtgcaggacctcggccagggactggacgacggccgagttggagatctgggcgggaaagaggacgaggccgaagatctccgccgagcggtagatgttcacgcgcccgccggccctcagcgtcaccgagctgggcacgtccgtcagctgggccatctcgtgccccctgaggatgccgctctggcgcatgagggcgtccagcctcgccccctcgtccaggacatcgtccagctccgggcgggaggtcagggggcggccggccagaaagctcttgaccaggtacaggacgcagttgctgacgcactggatgccggcgaagcggccgaacttgcagtgggactggttgcacgaggccgtgcccaggatgcggagggccgagccgccgccgcccccgcccgggggctccaggtccgtggtcctcgcgcgccccgagccccgcatggtctgcgtggccccccgggggcgctggcctcgtccgccgcctatcccgttaccgttcatggccctccgcggggcggggcgggcgtctcgaggcccctcttctggccctgggcctcacgcggctggccgcggccgccgccgagaggtaaggggggcgggtggggaacaccgccaccgcctcctcctcccctccgtctagtccggaggcggcccggcccgggggagcgcgacggttgcctcctctgtggcggcgactgcggcggtggccggaaggctcggcctcggccccggccttggggggcggcgtgccaaactccgcgggccttccctctctttctccctcctcctcgtcttcctccttcacgtcccagagctcgtcgtcttcccacctctcctcctctccctggccggagtccgagcccgaggaggcccggcggccgcggggcggcgtgaggtcttggctggcccggcgcttgctcgcgttcctgccgttagccaggagaatgaccgcgagtctctcggccgagaggatccgaaagggcggtggtgtctgcccgggaggcgcgggtgcctcgtcggtcagggccgggggtgcggtctggctctggctgcgggccgcgcgtgtgaaggccgcgaggaccgaggggcttctgggggtgacggtgatctgttcaggattcaggtccacggcgggcgtatatgttttaataggggtggtctccgggtcggcaggatcgcggggctcagtcagcgcccgacagacgctcttgcccaggtagaggatgtcgtccgcgcctagcgggggcacgttccccgtcctctccgggagccgcggctgcgccggggcgaagacgggcggcgggcggaggtctcgccggacgagggcctcggcccgcgcggcgtccatagcggggggctggaagagtcgggcgaaggtgtggtcccggaggcggtaggcgacgctggccctcagcgcccccgttagggcgtagcccaggtccagggcccgctccacgcgctcgttgagctcctcgaggtccggacgcaggggctcacgggcgttttcccacagggggtggtctgcgatcccccggctttccctcagggccggcaccaggtggcgcctccggagggcggccatgtcggcggtggtcagggcccacctggccgccgcgtcccggcacacgtcctggacgccctccaccacgcccttgagcgtctgcaggtccgtggagtagtagcggccccccgtgccctcctcctcgtcctcctctttcgccagggggatctgcacctgcccggtctccgcatcgtacaggatggggcgggagcagagcctctgggcggccccggtcaggcgggaaacgtcctccgagagggtctcgcctgagaccaggaagcgacgtgtggccaggctcaggccttccgccgtcgtgctgtatctcagggtgaagagcagggaaaagagtgaggtgggccagacgatgggtggtgccgcctcctcgcgccgctcctccgcggtggagagggtctcggagattacgcccgagacctcgcgaaccgcgtccatgatcctcaggatggcgtcgtgggaggagactccccagggccccgctccctcctcctcttcttcttcgttgcccctctcgcctccttccgtcccttcgccgccttccccctcctcctcttggggagaggaccgcgggggcgccatccggccgcctctctggtccggtggctccccgggcattggaggggccccgtcgtagacctcccagacggtggcgtaaatgagctcgaggggacggcgggcccgggtcagggcggccgaggggagactcaggtcgctgccgaaggagaccagccagcgcagggccgccagggagcgcgccttgggcagctcgttggagaggacccggcggagggcgggccaaatctggaactcgatgaaggcggccgggaaaaatgggctgcgggcgtaagcggggtccgctcgcgccgtctggccggccttcagggaccggcagtaggcctcgacgtctgcccgcggggcgaccgccgccgccgctaccgctgcccccgtacccggggtggccggaagccccccgggcgggatgggggcgggcgggatgggggtcggggccggggcctgccacaggcggccgtatcggacccacagcagggacctgaggagttcagaggacagaaccctggccacctgctcgtactcggcctccggtgggggcacgataaagatgcgcaggggggtgaccgcgtcccaggctgcggccgcggcgggcgagcgtctcctgtcggccgcctctgcctccgccctcaggtacgtcgcccgggcggcccgggcgaagcggctcgtggggctcgcggtgggcagcagccggaaaaagtgcagggcgaagcctgacaggctctcgagtagggccgcggtcgcctccagcccccgccaccgcgcgcgggacgcccggggcaccgaggccagcatcacggcgtagtcgcccgccgcggcctcgttgagacccgccgacagcagtaccgtggccacccgctcgatggccgcgggcgagaacgaggcccggccgccagctgcctcctgcacgcgagcggccagggcctccagctccgccgccacgccggccaggaaggcctcgatgaccgagtccggggtgccgtaggtctggtcccagagcagggcctcgtacacgtagtcgtagaagagggcccccgaggggtgcagcagccggagccaggcggcgttgaaggccagcacgggcacggccgccgccggggggctctgcccgccgctggcctccgcgtacacgcccaagatctccaccgcccggcggcgggccaggggcagcgaggccaccacgctggccagcgactcggggcggtggaagaggccgtcccggcctgggtgggcggaatgttcgcccccttcccctcctcctcctactccccctccccctgctccccgctccaccagctccacggccatggccttgatgtccgcggccgtgggctgctgcgtgccccccgccatcgccgcccaggggtagcggttggtctcggcgtagacggtgaccagccagcgtcccagagtcgtttttgcctcgttaaaagcgtagaatgacagcccctcccgcgggaaggcgtcccaccgggccaagtaggtgtcggccaccagctcctccacgaaggcgaaggtggccgcgggccccgagaccgagagcacctccccgcggccgtccttgaggatgcgccgatactcggccgccagggcccgggtctcgcggacgagccgcgagccgtccagcgggtccgcgggggcggaggcggggccgtcctcggcggggagcgaggaggccaggacgtccagcgccgcctcgaggtggcccaggccgtagctgcgccgcgagaaggaggccgcccggagcaggtagtaggcgtggtggcggagcaccgccgccgggtaagcgtggccgctcgcgagggtgagagtatttaaaaaatcgcgcaccagcacctgctgggccagatctccaagtccgaagatccccagctccagcggcatcagcgcgcgcagtcgggccgcggggtcgtcgccggccatcagcagctgacgcgtcacgcgggccaggcccccgtccacctccgtcaggggcggccgggcgccgagcccgccgccggtctcctcgccggcgtccatagtggcagccatgaaggtccaggggtccgtcgatcgccgccgtctgcaacgccggatcgccgggctgctaccccctccggcccggcgtctaaatatttctcagggggccgaattcgcgcgggacgtccggggcctgatcgcggagcacgcgcaggcctcctcggtgagcgcggcggccgtctggcgggcggggcttctggcccccgaggcggcctctggcggcgggggcggcagctttggctggggctggcggccgcccgtgttcggggactttctgatccacgccagctccttcaacaacgcggagcccccgggcacccccctcttccagttcaagcagagcgacccgttctcgggcgtcgacgcggtcttcactcctctttccctgtttatcctaatgaatcacggccggggcgaagccgccagggttgaggcgggagggggcctgacgcggatggccaacctgctgtacgacagccccgcgactttgtccgatatggtccctaatttcggggggctggtggccgaccgccgcttccacgccttcctcaccccagtgggccccctggtggagaacatcaagagcacctacctgaataagatcaccacggtggtccacgggcccgtggtcagcaaggccatcccccggagcaccgtcaagctgacggtcccccaggaggcctttgtggacctggacgcgtggctctctgggggcggggccccgggtgtcggtgagggaggctgcggcggtggcgccggtgggctggccccgcgaccgtgcccggccgacgcacgcctctacgtggcgctgacatacgaggaagccgggccgcggctcacgtttttccagtcgtcccgcgggcgatgccagatcatgaatatcttaagaatttattactcaccatccatcatgcaccggtacgccgtggtccagcccctacatatagaggagctgaccttcggggcggttgcctgtctgggaactttcagcgctacagacggttggaggagatctgccttcacttaccgaggctccagtctcccggtggtggagattgacagcttttactcagacgcctctgactgggaggtgattctctaagcttaacgcgaagagggagggggcttacaggagcattcgttccaaacccaccggcggaagacatggcatcccccggggagcgcgagctcctcaccaaattgattgatgagttgaaggtcaaggccaacgtggaccccgaggccgacgtcctggccgggcgtctgctccatcgcctcaaggccgagtccgtcacgcgcacggtggccgagtacatggaggtcttctcggacaagttctacgacgaggagttcttccaggcgcaccaggacgagctggaggcccgggtctcggccttcgcgcagagcccggcctacgagcgcatagtctccgccggctacctgtcggccctgcgctactacgacacctatctgtacgtggggcgcaacgggaggcaggagagcgtgcctcatttttacatgcgcctggccggcttctgcgcctcgaccacctgcctgtacacgggcctcagggacgccctgctgcgggccagacccgagatcgagaccgacatggaggtgtttgactactattttgagcacctgacctcccagacggtctgctgctccacgcccttcatgcgcttcgccggggtggagaactccaccctggccagctgcatcctcacgaccccgggtctgagctccgagttcgaggtgacccaggccctccaccggcccctggaccgctatctctatgagcgcgccggggtgggtctgggggtcacagagtatggcaaggacggcaagcacatcagcctcctgatgaggatgatcaacagccacgtcgagtaccacaactatctctgcaagcggccggtcagcgtggcggcctacatggagccctggcacagccagatctttaagtttctggagacgaaggtccccgagaaccacgaaaggtgcccgggcatcttcacggggctctttgtccccgagctcttcttcaagctgttccgggacgagccctggtcggactggtacctgtttgagccccagaacgccggaaacctggagaagctctacggggaggagtttgagcgggagtactatcggctggtgacggagggcaagttttgtgggcgggtctccatcaagtccctgatgttctccatcgtcaactgcgccgtcaaggccggcagccccttcatcctgctgaaggaggcctgcaacgcccacttttggcgcgacctgcagggcgaggccatgaactcggccaatctgtgcgctgaggtgctgcagccctcgagggagacggtggccacctgcaacctggccaacatctgcctcccgcgctgcctggtgaacgcaccgctggcggtgagggcgcagcgggccgacacgcagggcgacgagctcttgctgggcctccctcgcctctccgtcaccatgccaggggagggagctataggggacggtttctcgctggcccgcctcagggacgccacccagtgtgccacctttgtggtggcctgctccatcctgcagggctcccccacctacgactccagggacatggcctccatgggcctgggtgttcagggcctggccgacgtcttcgcggacctgggctggcagtacactgacccttcttctcgctcgttaaacaaggaaatattcgaacatatgtactttacggccctctgcaccagtaatctgattgggcttcacactaggaaaacttttcccgggttcaagaggagcaagtatgccggggggtggtttcactggcacgactgggccggaaccggactctctatccccaaggatatgtgggcccgcctctctgagcgcatcgtgagggacgggctcttcaattcccagtttgtcgccctgatgcccacctcgggttgcgcccagttgacgggctgctcggacgcattctacccattttacgccaacgcgtccaccaaggtcaccaacaaggaggaagcgctcaggccaaaccgggccttttggcgtcacgtgcgtatggacgacatggaagctgtgaatctggtcggggggcgcatctctcgcctccccgagcgcctgaagcggcgctacctgcgattccagacggcctttgattacaaccaagaggatctgatgcagatgtcgcgggaccgggcaccctttgtggatcagagtcaatcccacagcctgtttttgcgcgaggagcacgccgcgcgggccagcacgctggccaacctgctggtgcgcggctacgagctgggtctgaagaccatcatgtactattgccgcattgagaagtcagccgacctgggggtcatggagtgcaaggccaacgcggccccgtccccgcctgcgcccgagcccgagacgcagcgtccctctgatcgggcgccgcctcgtcccgtggagctacgggcaatcgccgggccggttggcgttctgtgcaaggggccgggggagggacctggtggggggtgtgtgccagggggattagaagtgtgctataagtaccgccagctcttctcagacgatgatctgttggagattgacggagaaactgcttgtgaatcttgtcaataaacgtttattgccatgtccaagttgttgtacgtgcgtgatcatgagggctttgcctgcctgacggtcgagacccaccgcaaccgctggtttgcggctcacattgtcctcactaaggactgcgggtgcctgaagctactcaatgagagggatctggagttttacaagttcctattcaccttcctggccatggccgagaagctggtgaactttaacattgatgaactggtcaccagctttgagagccacgacatagatcactactacacggagcagaaggctatggagaacgttcacggggagacgtatgctaacattctgaacatgctgtttgacggggacagggcggcgatgaacgcctacgccgaggccatcatggcagacggggctctgcaggccaagatctcctggcttcgtgacaaggtggcggctgccgtcaccctgccggagaagatcctggtgttcctgctgattgagggcatcttcttcatcagctccttctacagcatagccctgctgcgcgtgcggggcttaatgcccggcgtctgcctggccaacaactacataagcagggacgagctgctgcacacccgcgctgcttccctgttatacaatagcatgacagcccctgcagaccggcctggggcccagtggatccaggagctgtttcgcaccgcggtggaggtagagactgctttcattgaggcccgggggtggggggtcaccttggtggatgtgcaagccatcaggcagtttctggaggccacggccgatcgcatcctgggcgatatcggtctccctcccctctatggcacaccaccccccagggactgcccgctcacctacatgactagcatcaagcagacaaatttctttgagcaagagagtgcagattacaccatgctggtggtagatgatctttgagtcaggccctggctccttgctcaggtctcaggggataaattctcctgcctgcctctgctcttgtacgtcggcttctgcttctgcttgtgaacatggaaaccacacagactctccgcttcaagaccaaggccctggccgtcctgtccaagtgctacgaccatgcccagactcatctcaagggaggagtgctgcaggtcaacctgctgtctgtgagctatgggggttgtcccaggctggctgcggtggccaacgcgggcacggccgggctgatcagctttgaggtgtctcccgacgccgtggccgagtggcaggatcaccagggctccgaggaggcccctgccgccctgtcattcagaaacctggcctacgggcgcacctgtgtcctaggcaaggagctgtttggctcggccgtgcagcaggcttccctgcaattttacaagcggtcacaagggggctcgcggccagaatttgtcaagctcaccatggactacgatgacaacggttgtccaagagcccaccacacctgcgctctgatgccctacatgcccccggccacagaccggctgaggaacgagcatctgattgggcaggtgctgctgatgcccaagacggcagcctcgttacagaagtgggcgcgccggcacggcacgggcggggtgaaggtgacgcttaatccggatctctacgtgactacctacacgtctggggaggcatgcctcaccctggactacaagcccctgagcgtgggtccctccgaggccttcacgggcccggtgatcaaggctcaggacacgggggccgttgaggcccacgtcacctgctcggtcgctgctgactcgctggccgcggcgctcggcctctgccgcattccagccgtcagcgttcccatcttgaggttttaccggtccggcattatagccgtggcggcaggactgctcgcctcggcgggagacctgccattggagctcagtgttattttatttaaccacgactccgaggaggcggacaccagcacgcccacggagccagaagctaaaagccctgcgccgcagcctctgggcgcagaactccatcaacgccccagacacacggtcagtccatctccttcaccaccgcctccgcggacccctacctgggagagcccagcgaggccagagacgccgccgccagccaagcccagccactccaacgccgcgccggagaggcccctggccgttcagctcgcgaggaagaggacctcgtcggaggccaggcagaagcagaagcatcccaagaaagtgaagcaggcctttaaccctctcatctaacgccatgttctcgtgccagcagcacctggccctggggggctgcgtgttctgtctcggtctcctggccagcactcccttcgtctggtgctttgtctttgccaacctgctctcgctggagatcttctccccgtggcagacgcacgtatacaggctgggctttccggcggcctgcctgctgtccgtcctctggactctggtacccgccaagcgcgcggtgcgtgcggtcaagccggccatcttgctgaatatctccagtgctgtgctcttctttgccctcagggtctactctaccagcgtgtgggtgtctgccccctgtctgtttttggttaacctgcctctcttgtgcctgtggcccaggctggccatcgagatagtttatctctgcccggctatacaccagaggttctttgaactggggcttctctctgcgtgcaccgtctttgccctgtctgtggtctccagggccctggaggtctcggctgtcttcatgtccccctttttcatcttcctggccctgggctccgggagtctggcgggtgcccgacgtaacctgatttacaccactggcctagagcggagacgcagcattttctgctctcggggggaacatgcggtggcctcgctgaaggagaccctgcgcaggtgcccctgggacctgctggccatctccgcctcgaccattctcgtggtctgcgtgatgattgtgttgcatctacacacgggggtgttttttggcatctctagatacctgaccctgtttctgtatggggcaatggcctccggggggctctacctgggccattccatcatcaccgtgtgcgccatggccaccctctgcattctgaccgccgtggcggtgtatttcttccacgaagccctgggacccctgggcacgagtgtgctgtttatctctgtatttgtctactactttagcgcagtggcggccctcagcgcggccatgcgccacaagctgaagaagtttgtgaacgggcccctggtccatctccgtgtggtgtacatgtgctgttttgtcttcactctctgtgaatatctgttggtgacattcattaaattgtagcaactgatgtcctgtctctgagtgtacttggggggagtgttctgaccttcccatcattatcttgtgttaacgcgtggggatgggcacccgggggtgatgaactaggtgggctttcctgcacacagttggggtgggggacttctggtaacttcagcctcagatgtcttcacaccacacccaggtgaatgttgttccccacactcactgaggtgaacaccgttctgcttgtctgggtgaaggccccttcccagacccacccactcccccggccttcattttctctcctggctgctagccactcgccacacgtggtgccagtcccagcatttccacaaacacctttccattgtctcatccacgaggcacccttgtgcctactggggttgaattctagcccctctccagaaagcacactcagccggctgaggttctgctggcttccagtcccagaggatcccgactatgccagggttgctgaattctcgcatgcccctttttaactgactttcctgacccaaaggaccctgactatgccagtgtggctgaattctcgcgtgcctccccctttcttctctcgttatacccatagcatgacagtaaggttacacaggagaagtggattaaggttaaccatttattgattcaatccaggaacgaaaaacagccccagggatcccgtgcccggggagaggacggccctgaccagccggccgcagcaggcgctgcggcaatcgcgggtgtgagtctccactgcctccgtcaggagggagctcaatgtgccggggttgtaattcttgatgtagtggcccaggaactcaacctcttcgtatctccgtctgcagtgagcgttgatgtaggccggggccacggcggcgtacgctgccaggagacagaggggctgtttcacgtacgtgccgctcagggtctccaccaccttgttgtggcggaccgtggcacaggtgatgtagaagagctgtttcacaaagttgtagtctcgggtgttgggcaggaagcagggggccagctccttgagctttgtcaagatgaccttgctgaggttcatggcgcaggccaggaggatgtcttccgccggggccaggggcacgtcgccgtggtaggtggtctcctgtagccaaaagattgtctcctccagcatggccaccagtgtgcagagccctgcgttctggatggcctgcatgcgagcgtccagccaggtgtccttgttcgtggacttggtgaagaactcgcgtagtgttctgtagctcctgcgcagctcgtgtttgggctgcaccttctgccaggctccaatgtccggatgggcggccacggccagcatggactgaagaaaggggtcgtggatgggctccagggttagagaagccagagggctcggcagtgtgacaaaggtgattttgtcaaggggcttcaccaagttcacatcaaaccacggtttgttgggaagggggcgctggccgctatcttgcctccccggcttcctcgagctcttggcagcgcctccggatgtcggccggggacccctgggtgttctttcccgtctgggagccctgggtgcccttggtgctcttggagctcttggagctcttggagctcttggagctcttggggctcttggagccctaggcgctctgggggccctgggagcccgggcgtccagaggtgacctcgcttcccgtgaacgacgccggcgcttgccataactttcatccgtgcggctgtggacctggctgcgctgcctcttgcggcctctcgagtatggtgagggtccggcctcctccccatcctcatcatctctgcagagaggcgagcgatgcagcgtgactgtcttgttctgtaggtcccacttcttcctgggaatcacaaaggatgcagaggaggtgggcacgaccacgctggcgggctgtgcgggtgctgagctggccggcgtggggctggttggtgtggggtcttcatcctcctcctcctcagaggaagagatctccccgtccgtggagtctacctcaccctcctccatggggtccatttcgcagtctgacgcggcgtcagagacctcttcgagttccaggatgcggctctctggtgggagagagaaaaggggagacggctttgagcactcgtctcaggcaccgggcacccccggctccccggccccggtcactggacggtgcggcctcttacctgcggcggtctcgttagaggaggcctcgctgctagtctgagagagattctgtgagggaatcatcgtgtctgtgtctctgatgggccagtggtgatgtggtgccgtcccagtcggactcagccctatttatcattctgtgggtgggcacgctgatggagaaatgggcggtcggttgatttgccccacagcgaccggcgaagcactgactcataaaggtgaccgtgatggcctggggcgtgtaatacaggaccagaaacaccctcacattcttggagctggtcctgtggttatgcctcaggcacgcaaagttcctgccccggccatggcacacctgaactaaatttggccccgtttgctcaaacgtgacatggaggaactgggggaatttgtcctcgggcacagctgttgccagggtgctcatgaccgagggccagacgcaggagctgacccaggcgacgagatccaggcccagctgcccctctatcgcggcgcaggcattctccacggtggggggcagggtctcgcgggtcctcctgatcagatagtcgcggccatcgtcggagacgtggtagcagaaggttttgggagctggccagcccacgtgcagggagtgatgcagcaggttttgaatgttgagggcgttctccacatagctgcgcttgtcctcctcttccgggtgacagacaaagagccgcagctgcctgccgagggcgccgcccctgtccaccttgtaggtatgcggcagccggatgcaccggccggcgtgatacacgccgctgtcaaacaggggggccccgatgtctttgatcttgtggcgcaggcggcgcaggcaggccgtgagacccataagtttctgaagcacgcagacgaacccctgcacagcgctcgttcccacgatagcatggcctctgggtaggggggtgatgacccggaagcccagcttccccgtgcagatgcagaaggggagcacgtcttccatgttctcggagtcagggggtgggcatgccgatttgaaaaaatagacggggtgcgacctggacaccgggcccaggcggcgcatgaggcggagtaactcgcgcctcaccgtcaggcagaggtcatagatctcctccagcgtccagggcgccccccgggtcttgagatccagatccaggaccaggttgcagaccgggagcctgggattgaagtactcgtgccgggagacaaagagctgctcgcccaggctgttctgggaaaagtaggatggggtgtaggagatggcccgggtgagccgcgtgtccgggagccgacagtcggccgggacggagaccacctctgccagacgggccgagaaggggtcggcggccaccatcacaaagtatcgcctgtccgcgaaatggcagaggaagaccgggagccgctggccgctcctgagcgcggcgtccgggaggaactgctccttgggattccactggccgtggccggtcgcctggccggccaggcacaggaagccacggaggctggggggataggcgggcccctcctccgcgtgccagcttgcgagctccgccagctcccgggccacgtccgcgctgagcccggcccaggcccgggcgagcccatcctcggggtccgggtcccacgagtacggggaggggggctcggtgcggatgttcagcttctggacgcgcacgtgctcggacaggtaagtctcgcgggtgaagtaggtgcgcatgtgctccgcgaagccccggtccaggagcgaggggagcacgacgccccccactggcaggcgccctatctcccccacgctcgttatccgagagtatcgcttgaaggtagcctcgttgaaacactgcgcgtgggccagatagacgtagcgcacgaggtcggccgaggccagtggaaggcgccccctgtaggcatctatcgtcctagccaccgcgcggatctcgcgcgagtcccgccgcagcttttcgcgcgcgaagcgggcaaaggcctcggtccgctccgcccagacggaggagccaaagacctcccccagctcggccagggacgtgacggcggccaggctctgccccgcctcggaagtaaacagctccgtgaggtgcgtcagcgtctcaatcgtgcaaggaatgccccaaaaatagtaagcggccgagaccagcacgaactgggcctcgtgggagccgaaggtgctcaggaaccaccgggcagagacgttgaccctatagatgctgcgcaggcagcccacgatcttggggcgcagccacgccacacggcccctggcgtccccctgcggctgcttcttggcgctcagtgtgagcagctccacgagcggcgcgagcaagcgcagagcccccgcgcgatccaggtgggcggccagccggtccgcggtgagcggcgccacgccacgcaggaaaggggctgcctcctccccgggcaggcgcagcgtcaggacgcgggcgcagcggctccgggaggtcagacgcttcttggggaggtgaagctgcagttccacgagagatcccgccacgcggtggaggggcggggcgttgtgacataaacaaaacaggcggaagccctcctcgggccgcgagaggatggcgtcgaggacggcctccgccgtgtcagtgttggtggcaaccagggccttgatgacgacgggggtggacattatttaagacggggagcgcgcctatggggggtaaacagaacgatggccttctacctccccgactggacctgctgcgggctatggctctttggccgccccagggacagataccaccagctccccgaggacccggagacctttgagtgcccggaccgctggcgagccgagatcgatctgggcctgcccccgggcgtgcaggtgggcgatctgctgagaaatgagcagacgatgggctcgctgagacaggtttatctgctcgccgttcaagccaatagcatcacggatcacctgaagcgctttgacgccgtccgcgtccccgagagctgtcgcggggtggtggaggcccaggtggccaagcttcaggcggtgcgctctgtcatctggaacaccatgatctctctggccgtgagcggcatcgagatggacgagagcgggctcaaggccctgctggacaagcaggctggcgacagcctggccctgatggagatggagaaggtggccaccgcgctcaaaatggacgagaccggggcctgggcccaggagatctccgccgttgtctcatcggtcaccgcccccgcggccaccggcgcatttctaaaccccacccacgagtctgagattcccgccaccgtcctcgccccgccacccgtggtgcggcagcccgagcgctcgggccccgccgaactcgtcgtcgagccctctcgttaacgtagctaccaaacgcaacacctaataaacattttattgttgaaacaagggatacgagggtgtcattgtcccgaggagccaaacccccgctgctctctccccccctctagcgccgcccgtagcccgtttccccccagggccaggtccgggtcctcctcgaagatgcagctggcccacctaaagagggttctggggcccagtactctcgcgtcgctgtggggactgtagaaggaggtcaggtggtgtttgtggaggtagacgagctggcagaagcgccggtacttgttgaggaacacggtctgctcgccaaagttggtcaggctgacgctgaccccacctctgcgccacctgcacggcttaaccaggatgccctgcatggccaggcccgacctgccaaagatcgtcggcctgtggtgaggaatggggggtgggggcattgacagggtcacggagaccgtctggtgggggaagatggccaggtcctggggcagcgagacgtccaggcccacgtccccggggtactgggggcggctaatgggcccgcggtcctcctccagctggggggtggcatatctgaaggctgccaggtggatcttcagctccgaggggcgcagcgtggagttgtagcggcgttggttctgaaggatgagcctgagttcccccgtgtagccgggatcgatgatgcccacgtgggacgtgatgggccgcgaagtgctgccccacagcatgagcccgtggccctcgggaggtcgggcgtagaggcccaggtccacggccgtggtcctcatcgggcgcagcaggatggtggttttgttgaccagggtgagccgccccgcgctggcctgctggaagaggagcttgtcattctggaaggcgtagcgtatgcgtggactggcctccatggtgatgagctaaacagagagggacggccgtgctatatataagagcctcagacccgaccgtccgcactgagaaatggggaaggaccgagggaacccgtttgctaaggctgtgccactgatctttgccgccttcgcctggctcctggccggggcgctggtggccggcgaggccggtcccacacccacccccgcagcgccgggtgccgagggccgcgagcgtttctactcctaccagtgtaacgcggacacgttctctccctccctgaccagcttcgcgtccatctgggcgcttctgacgctggtcttagtcattgtggcctccgccatctacctaatgtacgtctgctttaacaagtttgtgaatacgctgctgactgattagacgggggatatttaaaagggctcgcattcttggccgcctgtacttcttctctgctcgttaaaccaatagcatgtcggcccctcgcaagacaagagtgccttctggcaaggggatggctgaaatgagcgtggaggacatggctgcccggctggctcgtcttgagtctgagaataaggccctgaagcagcaggtgcgcagggggggaacctgtgcctccacggcctctgctgcctccgccccattgcccccaccagagccgctcacaccccgccagcgggaggtgatgatcactcaggccacgggccgcctggtgtctctggcgatgcaaaagatcgaagacaaggtgcggaaagccgtggacggcgtgaatacccgcccggagatggaagacattctgaaaaatctgaccgttcgccttcaagtctctatgtctggggccaagggccaagcgggcccgagtgagggaacacgaccgcgagaagccgcggaccccgccgcctcccgacgtgcccgctcccgatcccgaggacgcgaagtcaagaaagtgcaaatttctgattaataaatctttattgactctaaacagcggtttcaaattcatcatcatatggggggtgggtgtaggtatggattctggacgagctgcgtcggaaggcacagtccgatatgaccagtaacagcagaagagtcagtacacccagagaggcccattgcagcactagcatggagagattggagccctcgggcttaattgtgggtgggaccggcactgtggcttgggtggtggtagccggtgggccagtagaagtccagcggggccgtagcgtgctagaattggtgggaggtggaggggtggttgcgttgtatactgttgtggctgagtttccaccacccgtgggcagttgtgtgcttcctccgcttccaggttcggtggtaacattggcctttccacctgctggggtgccagtgggaaccgtcgtctcctgactgctggtggccgagggcgacgttgcattttgggcaggtaggatggtggtgaccttagttgcccccggctctggggatgtggaactgtttccagggcctgccgtttggccggtggtgcctgggggaggcgttggagaagcggtggacacatgattcgtgttgggagcttctggtgtagtctctgttatatttttgctgcctgtttcatgagtagacgtgagcggtggggtgactgaggttgaattgacccttggtgtatagttggttcttggtgggcttgttaatcctgagccaggactggagctaggtcctatagaagatggataaggtgtatttgttatgacggacggggtttcagtgacgcctggagtgggactaatggacgctggcttggggctggtggcgttagggcttgtggctcccgatgggggactggttgccactgatgtagagccggagaccactgtggtgggtgaggtgtcgttaggggtggtggacactgaggtggggctggtggcgttaggggtggtggacactgaggtggggctggtggcgttaggggtggtggacactgaggtggggctggtgacattaggggatgtcataccagttggcgtggtcaacgtggggtcttcgggttgtctggttggagcagaagttaccggatcaggcgcctttgtaaaagtgacctggtaggcaacagcggtgacatttgtggctgcacgtgaaataattagtgttttcgaggttatgttgtccacctccacagtgagttcaaatgtccgattagacaaaaattggccggccattctctcgcagcctactggctgctggttgtttgtgtgtagcatccatttgcacttgaagtccttagacgtgtcatttgcccaggcccagaaggcagtcaccgtgacatttggtgcagtgttgttctccagtggcgccatctgtagttgatacgtggcattttgtccagtgtataacaccacttgagtttgcgccggcatgtcctgaatagtgggcagtttgtcagagaacataattggcgccttaaggcagaagtccccatgctcagagtctggatcctcagtatacataatatagagtgtactttcattgcctagaaaacgtggcacggggcgcggcattaaagtcaggttgtatacgtagccgccaatgggcgccggcgtggtgctggggcttgccggggttagaaccccggcactcgcaaaatgaggcttgtctccatcgcatttaattgcaaagttgttgtttcccggcaaaactggtgaaatgggatctccctcattccggcacagcatgtcaatcccctcgcttgtcatttccatatgtagagaaaatttggtcgccagaggcattgttggcaagcttatgggaactgatatgtccattccctgtgcccttaccacagctgttatattggtagaattacagtggttccacacaacagggggcatcttctttggtataagatagacgggttctttcatttgaactcgctggcaccacaaaatgtcaaatgcgtccaggaaataaatgtatagagcttccatgctaacgtgctcctcatccctactgacattaattggaaatatgatggaacgcattgtcaaagccatggtacgagcaccgagcaactcaagggagaagagactagtaacattttcgatgccaccactggcatggagtggttggtaaatgttttgggtaataggggtgatcggtccaaaatagagatccagatgcctattagcttcaccaaccttgaagacgatagagatgtttacatcggcagtgcagacctcacacttgggataaaaggggaactgcagcatctctatattgaaaaatccaggatcttcaccagcaaagtggaaaacactctggagggtgtattgacataacagaaaagccgcttccattgtttcagtgcagattcgcaaacagccccttctttaataggcaccgaaggggtgcggtgttggtgagtcatgcttttgttgcagacaaaaatggaggaagaaaggccgggccccttggcaccggatgacaacatggaagaagaggaggttccatccacctccggtgtccaagtacaggcgtcaggtatggataaggacattgtcgtcatacctgattctgaaagtagctcgggggaggaaggagagaggttaccagattcaccaccgccaccaccacagaaggggaagaagagaaaacggagaaggagcgatgacgccagtggcacaacaccgagccagccaccttgtgtcccccagctggagcttggggtggtagaagactttggtgggccacaacatcaaactttaggaaagctccttcatgctgtcagtgccacgactttggtgagcaaccaccgccttgttgtgtgatgcgtgttttcacccatcgctcaacaaggcacgcgtgtgatttgttgccattaatttcagcatcccaacacgcgagccatacgcctgttttttggatccagtattctgactacccagaggttcctggcttatgccatggctgtccgacagggcgttagagatcgtcaggcaattcaagctacgattaaatgtcagaactcctggaaactgaccaccacggcctcacgctggagaatgggttaccggaagcacagcagctggatgtacagctattcggatgatgtgcggcgccctataagaattgctatttgtgtaacatttgcatgtgaagaaggcgagaggtggaacgtgtcctactcagcaggcaccttcaaaccggtgcgggcaagcaattatgatatggtgtggctaaccacacaccggtgtgttggtgagctagtggaactctgtgatcctatctatcatcctttctttggctatctgtgcattttccgctgcctgaaattcatctgggaaaatgtgttgttgcccgagcaacgcgtgccatttatgcagttcctaggctttctacaaaagacggataacatctacatgaagcggtttgttcaggaaggcctgctcacctccaacatacaaacgccgtggctgagtgaagaccccgagggagcagtggacagggggtggacacaagccttaataagggggcgtgtgttaggtttaagtggtcttggtcccgcggcagagcccgacaaagatgctactggtgaaacgcagcaagaagctgaggacatggagagtgatgatgaggatgagattcctcgtattgtgtctcgggaagaaaccaagagaaaagatgcacgcccccctcaatttctaacacgcacgcacaggctaattcttgagagaacaggggggcttacgctcagggccagaaaggccctggagggacccagtgagccagcctccggggggccaagacgcaggaaagagtgtgcaccagaacccccactcacccttagaaacctccacatactagcagggatgttccgtcagcagtttaagggagtttgtgcaccccatgtgtcttcacccccattagtttcagcggtcgagatcccaaggccccaggcagtacaggatctggaacctgggccagtacagagtccagaaccggggccagtacagagtccagaagcggggccagtacagagtccggaaccggggccagtacaggatctggaaccggggccagtacaggatctggagccggggccagtacaggatctggagccggggccagtacaggatctggaaccggggccagtacaggatctggagccggggccagtacagagtccaaaaccggggccaacaccagccccccccactgggcattttgttaggccctgggaggcagcaccgtcacaggttcccaaagtagccccggctacacatatgccagatcccatggcaggggcttttgacccaagacctgctccattgcctagcttggccttacttacaggccccgccccagaaacatctagctccgtccaggtgtccatgtcatacgctgccccaagctgggccccaacacctgtccaacccgttgtccccattccaatacgttgtggtccacctccggattactttacttggtatagctccagaggcaggccccagagatacccctacacacatccggtggctgtacaaccccctgaagttcctatggcgcccaccgaggctccgttgtcttacccggccatgcaggcggggagaattcctgtaatgaggccgccgtcccgatcaagggggggtaacgaatgggagacacgaaggccatatagcttgcagaggccaaggaccgtgcgtgcgtgtctcgctatgatgcgctctggtactcttggcagaccaccccggggtgaggacactgagacgtggttagaacgtaacctgtctcaggtcgccgggcttcggttggccacaggaacttctgctttccagggtctcgatcaggcccaggttccagcaaccccaccacagctgccaccacgagtgggagcccccatgtctcgctccatttgtcggcgggctcccgtaattccagcacctcctcagctgccccccatgcctggccagtgggtctttataccagaccaagaaccgcttcacgaagcttccgttagaccacgtgttagtcagggcccttaccagatgggtgcctttaggcaggtacctcgtgtggaatctccgccccctgaacccaccaacacccacgctccctcattttttggcgttacccaatataatgtcagccaggtgagctttgatgaggatattgaggagacggaaagtgcagatgggtccgaggcttcagagttatgtgaccctattgatttgtcaatccatggcaggcccaccccaagaacccccgaggtgatttttgtggcggaggatggccagaatgtgtcaggtgcgggtagtggtgtggttttggtgtcagctctggttcactccggtcagggtgaagacttgcctgaccttgaggatcccccagataatgaggaataagggctgtgtaggagcagcatgttttcccattaccctgctgatatttcccaggcaataaatctgctgatgctgaattccaaaatgtcttctttgactttatttcataacttagtccagaggaaaggccatactcttgtaggttattgagatatcttatctcaaaacagttaaacccgccaaaatgaagaaagcttggctcggcagagcattgcgggatggggccgcagaggcaccggattctgaagacccagattcaccacaggatggagagagaggtaatatgggtgatcccgggtctaatcccagtccacgtgaaactgtgtccccgggaccgtctactgccagcgagcaaatgttgcaacacgagcccatggaagagaattcagatgatgacgtggaagaagtgagccaacgggccagaacgcccccacacggctatcaggaggccgctgctctctggtcggctcggtatcgaacgtttgtgaatgtaacaccggtgcaggcgtcagtaatccaagtagtccacgctgcctatgatagcatgttggtaagaggcgcccagaacatacgtgtccactagtttttattggctatctggcctgcattaatttgctgttattggtttcagtttccctcatcacggcatgtagccggtttgttctttgatgaaaatctgacaaccgaagagtttatatggctctgcatgactgtgcgtcatagatgccgctctgtccgggaagaaccatggcccattgcaaaccaaagacggtggaggatggtgtccccgcagagaacctggcccatgggattttgtacgaggcacctcatgatagaaaattttgagctttggggcacttcaacagtccgggtctatctgacggccactttagggtgtgaaggtggcgagcgccacagagtgacttacagcgccggtaccgtaatgccgcccagaatgtcagaattttttcagaagcagttgacatccttttcgtttgcagattatacccggtcccattcatcgcccagataccacatgttctttgatttagtgagttctggtaggagactctatccaatatggatttcaccatggtctagggatgagagaattgcctttgtgcagttcgtaggatggttatgtaagccggaacacagactcatacactactggtttacacagtgcactgggagacgccgaccaaccaggccgtggctgaaaccgcatccgcctgatgttccttatgaaaaccccttaacatccagagatatcgctattgcctttgcctgtggactagccatggagctctatgcggtcagactgccaaatgatcccataattgtcgaagatggaagcgatgaaagtgactctgagagtgaatgtgacccggatagtgccgcagaggccctagcgagcatgccatgtataattccggatcctgtgtacattcacggcagaccaaaggtttttgccacgagtgcagcacttaaacaaagaagaagatttagggccgtcttgtctgacccgagtgtagttgatgcacttcgggagcaatgcaaaaagcagaaggcgggacaggcagagcagatagcagccccaactccaccaaaaacaacacaacccacaccacatccccaagtacccacagtgatcctccatagaccacatgcacagccactggaaagtcagaccgtgccaccaagagccccttaccccctggtaccgcagccccctttgccacatatccaagtagcacctgtcatcgttcaccaacctccagcgcaagtcagtgcacctgagtccttactagacctacttgagcaggataatgaggatgctgaacaacgggttatgtcaacattcatagaacgcagcccactgccccgtccaatggccacaagagctgctccctgcgtttatagggaagaactagacattgaagaagatgagcccgccggattggggcaaattcagatacaggcacacttggccaccatttgcccagggtacacaggcaggtccaacctcaagcctcatggctcaacccactctgagccaccttcaaccccccgggcacatatacccgccaattcagaccccaggcgggccacctttgatgtcttgggcttcagaagcccggattggccacccaagaactggaccaatgcgaaccccagtacggccaatgccaatgcgagccgccccatatcagctgccccaggccgggcccgccccccagcagcagccccaggccgggcccgccccccagcagcagccccaggccgggcccgccccccagcagcagccacaggccgggcccgccccccagcagcagccccaggccgggcccgccccccagcagcagccccaggccgggcccgccccccagcagcagccccaggccgggcccgccccccagcagcagccccaggccgggcccgccccccagcagcagccccaggccgggccgccccccagcagcagccccaggccgggcccgccccccagcagcagccccaggccgggcccgccccccagcagcagccccaggccgggcccgccagtgtgattgtgatgcctccccaggctcctaaaaggggcaggcctgccagtagaaagccatcacgtggagctaaactgcgcccactagtgccaactcccgacccgcaagggggacatactgtgcaggcacctgtcttttttccaccggccagcacaccttcttcacaactgcccagaaagctgggtttcgtaaggcccgcgggtgccagcacggcaccccaggcacccacccaagaacccggagaacggaggcactgggaggaaattctccctcccaaacgagccaaaaccgatgccaccgtagagtctcagcccgcccaaggagagcaattgccttccttttccgttatttgggagaatctcagtctgggcctagagcctgctctaggccagggagaattcacgaccgaagatggggccaatgaggggaccaagggtggcgccgccgaatcttctcctcgtcttgaaatatcggacgaggagttctaatgttctcggtggacatgaactgggattaaccctttaaaagataactaatgaaatgagggacagtgggcaagataacaagttactacaaatttagatgaggcagcaattttacatatttacagacacaacaatggcatcacaaggagagggggacgatattcattctacccaagaggagccaggagacaatgtacagcgcaccggtgagctcgtcccggggacagggtcacaggccggcgagccccccatggggacgacacagcgggatgagtgtacagcagagaccggccaacaggcaggcgggggggaacaggaaacctggatgcaacacgtgcggcaaaggcggagaagaaggcacgagagggctggagaatacttaggcggagcgggcgtcgagttgccatggcttcctccatttgtgactaggccgtattcagcggtggagctcaggaatgccgcctatgaagctgtcgtggtgagcatgcctctaaagcgggttatgccacgggggcttcccgaaaccctgacctgacccacgtctcccttaattttagcaaccttcaacacatgttctagccaacctgatgttcgatagaaatgttgacccacgtcaaatggtgtgctttatgatggcggccagacagcggtgccgtgatctcgaacgcggtcccagcacagcgcctgggtggcgacactgggtgctcacctctcccagtgtaacgtggtatatgggattccgcacggcatcaatccgcaccagaacatctgcgcccgatatgattggagcagacaatgtaaaggtgatggtaatgatgggatgccacacgggggcaagaaacgaaagggccttctcggctcaggtctggacaccctacctccccggggcgagggagcaagaaagattcagccgcgaagcagaagcgttctctcaaagacggcgatggcaaaggcggtacagagttatttttgatccctatagtgagtcgtattattcgctgcgccacatctggcttcacagactgcagagtgacgcccaacttttcgactttgcccgtttcatgggagccatgacacgttgcggcaatccggccttgaaatactggtggcacaagtcaatcgggacctacaaaccgagcgtcccgtggtacccgagacctaatgagcaggcctttcacacgtgccggggcctgaattcagacgatctcaagaccacgtttagagaggcacagaggttgggcttaaggctgctggagagggaagaacccgacattgatgatatcgttgaaacgagcagcgaagaagagggcggccgagattcggactcgcagatggagtcctcagatgacgagctgccgtacaccgcaccaggcatggagataccccagaggaggcccctcattttcatttacggcctgccttggagaacacggccatggcccacgtgcataacgcaccccgcggtgccatcactgggtgttgatgaaggcagttccgatgagatggaacaagattcgagcctcccggcccagccaggcccatctgacaatccacctgtacccaccaccccacccatattgacccaggttgaacacacgaccgcgatcgttcatcagccgccccaatcgccccccactgaggcaatcgaggacgcgccacaaagtgttggggtgccaagtggtgcgtgtgtggttacaactgcccatgatatcatagaggtgattgatgttgagtcctccgatgaggaagaagccccagcgcgaaagcgtcgggctgtcgtgtccacaggcgaggtcagtgggactttgcccaaggtgaagccatccaagcgaggccggccaaaagcatcgcaacctgagaagcaacagcaaaaatgtaaagatggagctcctgcttcgacgcaacacgagaagcaacccaagtcctccgccccaccctccaaattccgtgaattactgagggaacgactgatggagcggccatttgggccaaagccaaagagcttttgggaaatgcgggcaagtcgtgatggtagtggcaaacaaccaccagcttcacgccgggggccaactctacaacgtcctacgcggctcccatccgtctttgtgctcccctcggtgcccaccacgagacctcgggcctcggaagagtctcgcaagcacccattgtctgccaagcggccgacactacattcagagcagctcacgtatgaggattcttcaccctcacggcatgcatgttcagcatatgatgatatagagtttgatgttcagtattcagaggacgatgaggaaccaaaaccccaggagccccaggatacaactgatgtggaatcacagcccttacaggatccacccccgggcttccgggaggcacaataccaatcccaccgggctccattttccggtcatgaggagccgtggccacaacaccagcaggctccatacccgggctactgggagcctcggcctcagacccaacatccacggcaggctccgtgccaaacatatggggagcctcagccccagccacaacattcccagcaggctccactcccaacatatggggagcctcagcctcagccacaagagccccagtgccctacataccagggttatgtggagttgcagccacaacagccccaggccccgccgattccctacctgggatacagggagcctcaggcccagccacaacatgcccagcaggctccacgccctgtagcacatggggagtctcagcctcagccacaagagcccctgcatcctccatacctggattacagggagccgcagccacaacagtcccaagacccactggctccatacccgaattacggaaagccgcagacacaacatccaaaggccatgccgattccatacttgggatacagggagtcgcagccacaacagccccagcccccaccagctccatatccgggttacggagcaccacggatgcagccatcgcgggctccacacccgggttacagggagatacagccccaggccccattggctccatacatgcatccacgggtttcagttgctcaatatccaggtgactcaggtccctggggtttacgggctcaatatcaaagatataggcactcgtgggcatattggtctcaatatccaggttatgggcctcctttaaccccctgggcccaacggcctccatatccaagttatccatgttcacagcctacttgggctccatatcccaggccagcacatacccatcctttacaaccacagcccgtaccaccgacccccgagcaaacacaggcccaacagttgccctattcacagaccccagattcagctgcagcactgccattgttatctacacctcgtgaagcccctttgcgccccattccgacaagactcccaactccttctaccccgttacaagatagcatgaccacgggtgcaactgcaccgggtacttcgcacccgaacgtaatttttgccagtaacctcagtcaaggggcctttacacaagtgggcaccgagggccaacatcaaaaaaggcctcgtttggaaggctctcatttccacacaaacttagacaaagccgccggggaaacccaggccgctgaggttattagtgagggctctgacgtttctaaaggccaaaaggacacccagcatacagactatgatgcatctacagaaagtgatatagagtaaaaatttcatccgaatacccagccctgtgtctgggaccccctctagtctcaatcttttctatgactaacacataaacagggcttatgaaatgaacactcgtgatgagaaactggaactttactagatttactgacataagtacaatgagagccaaagatctgataaacttgataattgcccccatctaatgatgtccagttcccctctcctaccttgtacactccaacccaaagagactctgaagcttttaaatttccagttaccacaggaaggatgttggccgttggcccataaaaataagtgcactcatatagctcagcacaggccttaaaacatccatcccagggatgcttctgtggggtgaaataaaagcagcgcttgctaaatgagaatgtgtacttttgagtgttgcaatatgtgcagttaggtttggtgtaattctcaggtaccttgaatgtgtgcagggtgggtttccaatgtggaaggtctatctctggctttccatactcctgctcggctgtcttgttaaagtcaattggtggcgggggctctgcgggccagacctccacctttgggttggccacccaggtgatggctgcggccgccacctgtcctcctctcaccctgggtggcaaatagtatgccaggaggagaacaataacaagggccagggcggtaaacagaggcaccctcacctgcttaaatgaaaccatggcaacaagttcaaagcattccaacaggaagtcatttattaatattccattagtaaacggggcgtgatgcaagcggggtttcgatggaagagggtaaaattatataagatcttcatggataatgtctggtgtccgggggataatggagtcaacatccaggcttggacacatctgcttcatcaggatgcgtagccgttcattttccgatgatttggaggcggctacctgtgtggaaaaaaatgcatgtgtttagcttggaaatttatgcacgggggggagatatctgccacccgctcattcaccacttgaacagtcttgtttcattgtacctacctcacggtagtgctgcagaagattcttgaacctggctcggcattttctggaagccacccggttcttgtaacgctttatttcaagttcagagccacactcgtccagctacaagcaagggacacatttacaaatgtgtcctgcccagctgcccctcaagcccagctgcccctcaagcccagctgcccctcaagcccagctgcccctcaagcccagctgcccctcaagcccagctgcccctcaagcccagttgcccctcaagcccagctgcccctcaaggcccagctgcccctcaaagcccaggctgccccctcaaagcccagctgccccttcaagcccagctgcccccctaccgattctggctgtggtgttttcctcggacgtcgtgccggtacaggcgccgtggcaggtggttgggcatccacctccaccaatggctggccttcatcagcttccgggcaggcacatgccgcttctgctctcgttacagcagtagaattgtctccaggttgtggtgcttctcccccggcttggtgaaactgttgaatctgggtcaagtctgggacttggagcagctgaggtgctgcatatgtcggataaagattctcaggattagtctgaaaagcaggaaaccactgcccagtaggagcggctgaaaggtgataagcagtgggctgctgaacatgtggcagtggctccgacagcaccggccaaagcacacaaggcagactagtagtaggctgttgcgatggtccacccaggtcctgatagactctagtagccgtttcaaaggcctgttggaacggcacctggtatggtgcgtcagctgtatatttcacatcttcaggaggcaagctggggtccatcgcgagaggcagagataggaaagatgaccagcaagttgcaatagttagctacctttgtgacagctcctcttttaaagccaaggcaccagcctccccagtgatgtcatggtttgggacgtgccaaaattaggtgtgactagaaggaacattagcaatgcctgtggctcatgcatagtttctaaaagaggaggtagttttcagaagttcctaaaataagctggtgtcaaaaatagacaaccctgttgaaagatgcatggcatgtagcagacactcatcatttagaaatgtatctaagatttcattaagttcgggggtcaggggggaatccaagtttaaatcctctgtcatggtctctaaggtggtggccagttcgtccaattgaccacaagggggcgtgtggtcaagagccatatgtccacgttgtgcagccccttccttctggggaatggcaagtgtcaccatttccctcagggccttcacggcctggctggtctcctcgtcgggatcctccacgccggtcccggcttctggcgtctccaccgggcatctggtcacacggtcgggggcacgtggggtggcaggaggcagccgcccgcctcggaagctggcaccggggcaaagggccagagaggcagacaggggctggttggcccacggggaccctggtggatgaaagggcctgattctctttggctggcgtgtctctcgtccatcggccatgtcaggcgtgacactgtccgtttctgtgctaggtaggaacatacccccccggccatcccagaggcccttcttccttttaacgggaggaagaaaggtgggcttcgaagggtgagggaagatgggtctctcatcagcctcgtgggtcaacatctcttctcgggccacgctggaactagggtgagagagccgcggggctttaccctggggcctggatggcctgcgggactttgccccccccaagcgaggcgcctccgtcccaacctcctcccggtcactctggtcagtgtcgctgttgccagtgtcgctgccactggagccgctgtctccgtcggctcctccagcttctggatcaaggggcatgtctaggccaggggggctctcgagctcatccgtgaaatcatgaaacggtccgctttctgcgtggtaggcctcggagtgagaggcccctgcatcatccctaatgagctcctcctccagtccctcggagccagacaccagaatcggagtgtccaccgccttgggcttggttgacagcaggcacgtgggcagcacggcgctcacgtagctcctctgtccggcatggctggagtaggaggccctgggcagggtcttgatcagggccctcacatccttcacgtcatccgtcagctggccggtcttggatgagaccattgtctggaacatttcatcgagaactggatatgtgaacacccacttgcaaaaggccttgaacttggagcttaggaggccttccttctccatcctcatcatgtgttcaaccacctgcctaccggaggccatgatggccgcccggtccacgcctagcaccttgctgtaggtgtaggcacgcacccgacagtgtttcaggagcttatacatggctgtgcctatggtggcagggatcatcaccctgttgctgggggcctggatgaaaaatctgtctgtgagcacaatcaggtgatctagcacataccgcatcacaatggggcacgcgatggagcaagcatcgctaccggcattctcggcccgtctcctcaccctgttgtttcggaggatggaccagaaattgcagatgttgagcgtggccatgagcccgccccattctcgcccgtgggccttggcatcatttataaatgccttgcatattttgtaggatctcagagtaatctccacgctcccggctgtaaattccttgttcaggacgttacagtagtcagaaatcagagagaccagctgcttttttatctcgggagtcagtttcaggaagtcttctaggccgtcctttttaggcctcatggctagtaacagaggaaacatcaggccgttaaaatccttcctccacgagctctacctgaagcactatccagaggtcggcgatgttgtacatcttctcaacaccatcggggtcgactgcgacctcccaccaggccacccactgctcacggcccacagggggttgtttttggccagggtcctacaggctgtgcagcagcacagactgctggaagacgccatcatccccaagatcctaaagaagctggcatattttctggagctgctgagctactactcccccaaggatgagcagcgagccatagccgaagtcctggaccacctcaacatgagccgggacatgggactggatgacaagctgtgggccctgatcagaaccctgcgccaggagaggcaccacgcttctgtgaatgtggttctgcctgagagcgactacacggccctgtccctgcagtactacgagggcatctctctgggcatgaggaaggtcgttgcagatatctgccgaagcggctatgcctccatgccctccatgacggccattcacaacctctcacaccagttgctaatggcctcaggtccgagcgaggaaccctgtgcctggcgcggtttctttaaccaggttgttctctggacagtggcactctccaagtttcacagatgcctgtactacaactacattcatggctccatagccaccctctcccagctgctgcaccttgaaatcaaggccctctgtggctggattatctcccaggaggggatgcgcatctttcaccgcaacaggccgctcctcaccctctgggagagtgtcgccgccgaccaggaggtcacagacgccattcccctgcctgactgctctgaatacatcgacctcctgaagcacaccaaacacgtcctagagaattgcccccccatgccatatccataataaagtctcttaccttctgtttgtctagtctggtgttgtgtcctcgatggtatgctatgacaaaataggctggctgacatgggctattgggcttttatgagccattgtcaggggcgggacaatcgcaatataaaacccagaccatcacatgggagctttaggcgactctgcatcaacatcgcttcaggatgagtgggcaacaggaaggctcggttattttggttcctgaacatctggcccgggaggtgtctcagctattgagcgattttatctctgatcatgatgacgctcttactggcagaaatattgcaggcaaaatccggcgtgtgctgggtcgggcctctggggaccataatctgcctcttctctcctccctgtttgccttatggaatgctctcccaggctctattaggcaagctgccagggaaaggctgatattaccaggcgtgcaggccctgaccacggcctatggcttgtgccggggggtcattccggaagagacatctcccagtgatacacccgaaactttgcttcaggctgttgtgacaggtctgcaaaaattgtggttggattcctgcggctgtccagagtgccagcggtgtgttaggggcttaggggccctgactcccgggctctttgaactacctaagatcatccctcacactaagaaatgtaatcctgttaatcttctgaacacactggtacacaaagccgtggctttgggtggtcaggtgaaacatgattatgacccacggggcttaaccccaggcttggacgaaatccctgacctcgatgactcggatggtgtcctcgcacgcaccttactggcggccttgtttcacctctctctgttttttattctcagagattatgtaacaggagactcccagcacctgaagcaggcccttggtggccaatggctagcagaaaccggggacactctaccggtggacccacaaacacttagagactacattgaacagtaccgcaaagtggataatcacttttatttccccacgccggggcctttgaaccctttccactttcctgaaactctgctggggcgcattttggtgaccgagcccaacgtgtgtgccgcgaagcacgttgtcgcgttggttaaacagggaggcgggggaggtgttcctcgaccgatgctaccggcactgccccaggtggcacccccggagaccgggcggccatgctctcatgcaacgcgcaggggaatggagagctcacagcggaacttgggcccgccactggggacttccccagatgtttccccaatttgtcccgccgcttccccggcaccctccgagccccaacgtcccggcaagcctggactttcgctgcccacatccttctcttgtcccgttgcccctgtagcatcgttgctgggtgactacgaggaggctgcagtggagtcctcggactcccggtccctaccatccagcgatggggggatggaggagtttgaggcctggttggaggcccaagaggctaacttcgacgaggtgcagcgggagttttctgggttgcgggtaaccggtgaggaggcagatgatggttcctacgatgagggtgaattttcagacttggatctgtctgacagcgaccacgagggggatgatgatgagggggatgccgatgggggtgggagtcttcgctccctgtattccctgagcgccatctaataaacatctctaattgctctgttctagtgtgaatcatgtccgacggaaggggcccggggaacggcctgggatacacgggtccgggccttgaatccagaccaggtggtgcatcgggctccggcagtggcggaaatcgaggacgaggggcccatggccgtggacgaggacgaggcagaggacgggggcgtggaggtggaggagtactaggcgaaactggagaatttggtggccacggttctgaatccgagacaagacatggcaatggccaccgtgacaaaaaacgccggagctgcgttggctgcaaaggtggcactggaggaagcagtgccggtggtgctggagggaactcaagaggaggtggtggagcaggagtgggttcgggaagaggtgcagggggaagcggtggagcaggaggtggtgctggagggagcctaggaggcggtgcaggggggagcagtggaggcagcggagcaggaggcagtggagcaggaggcagcggagcaggaggcagcggagcaggaggcagccgaggtagaggccgcggcaggggaggtagtgcagggggcagagggggaagaggaggaggcggtggaggaggcagccgagggagaggccgaggtagaggaggaggcagtagaggtagaggccgtggtcgaggacgaggaaggggcagaggggaggggcctagtaagggtgaaaaaagaccgaggagtcccagtggacgttcatcatcccagtcttcatcacgtagttcttcatcaagtcgttcatcttccaatggttctgactcaagtgactttccagggtttccagggcacaggccacttccaacttcttttcctggatccccactaggtggttacaggggaactgatggaactgatggtggtgatgaacaacctcccggagcggtggagcagggcccgggagaagacccgggagaaggccccagtcgccaaaccacgacctctggtggccgaggcagtggtaaaaagggagggtggtttgggaggcgccgcggcgagggtggcagaggtttcaagaagtttgaaaacatggcaaaaaatctcaaggtgcttctagccagatgtcaggctgagcggacgaacacaaccggtaactggccatttggagtttttgtatatggccctaaaacttcctgttacaacctgaggcgatgtattgcctgttgtataccagagtgccgccttacgcctctaggacggctgccatttggctatgcacccgagcctggccctcaacctgggcccatgagagagtctacagattgctacttcatcgtatttttacaaactatgatatttgccgaatgtgttaaggatgcccttcgagactacattatgaccaagccacttccgacctcttctgttcaggttacagttataacatttgaggaccccgtgatgcttcctgtctttttccctccacaccttccagctgcggcagtggcagcggagggaggtgagggtgccgagggagatgacggagatgaaggaggtgagggaggtgacggaaatgaaggagatgagggtgcggcggggcaggagtgatgtaacttattaggagacgccctcaatcgtattaaaagccatgtattcccccgcactaaagaataaacccccttgccatcatgcgtgctgttagtgggtttctgtccgtctttctcgctgtcatttttgtcctccccacgtggggcaactgggcctacccatgttgtcatgtcactcagctccgcgctgaacacctccttgcgctggaaaacatcagcgacatttacctggtgagcaataagacctgtgatggcttcagcctggcctccttaaattcagcaaagaacgggacaagtcagctggtcattagccgctgtggaaacggactcaacgtggtctccttcttcatcgccgtcctgcagcgcagcagttccgccctcacagttcagctccgtgaattgttaaacaccctcgagagtatctacgcctccttctcggtcgaagacctgtttggggccaacttaaacagatacgcatggcgtcacgcgggctagacctgtggctggaggaacatgtgtggaagaggaaacaggagaatggtgtcaaaggtgaaaatctgctcctccccgacccatggcttgatttcctgcagctcagccccatcttccagcgcaagttggcagccgtcatcgcatgcgtccggcgcctgcggactcggaccatcatctacccagaagaggacatgtgcatggcctgggctcgcttttgcgacccctccgatattaaggtggttattttaggccaggacccctaccacggaggacaagcaaacggcctggcattcagcgtagcatatggcttcccggtcccccccagcctgaggaacatttatgcggagctgcaccggagtctgccggagttctctcccccggatcacggctgcctggacgcgtgggcctcccagggagtattgctgctcaacaccatcctcactgtgcaaaagggcaagcctggctcacacgctgacattggctgggcgtggtttactgaccacgtaatctcattgctctctgagcggttaaaagcgtgcgtgtttatgctgtggggtgcgaaggcaggagataaagcctcactgatcaactccaagaagcatctggttctgacctcgcagcacccctctcccctggctcagaacagcacccgcaagagcgcacagcagaagtttgtgggcaacaaccactttgtcctcgccaacaacttcttgcgcgagaagggtctcggtgagatagattggagactgtagcggcgccgggatggccatgtttctaaagtctcgcggtgggaggtccagaagggcccagcggctcctatcagaggacgaggaggacacctccctgggcagcggctacaccctggggtctcagacctctcagtcaacccaggatgaagatgaggaggaagacctgagcgagacagaggagtctgagtacacagacgatgatgaggagattgacttggaggatgaatattccagcaacgaagaccagtcggagggcagtgacagcgacccctcgtggcgtccttcggattcagaagaatccgactacagtgagagcgacgaggaaggtgaaggggcctctgcctctcaggcttccctgtcttccagggcctcgcaatcctccaccacacccacttcgagcactacaaccaccccatccttttctcggccacgagcccaggtacctgctaggccaccaccccccgtgctgtccagacagagaggaccggcacctccaaggccaccggctccggcccagaccaaacagcgacccccggcacctgcgaggccgcccgcccctgcgcctgttaagcagcgtgctccgacccctcctaaacccccgagacttacgctcaaagtcccacccccccgccgcccccctccgccgtcctgtcccaatagatacaaaaaacacatgatggttaccacccctccgatccagggcaacaaaccctacaactggccatggctgtaaataaacactcagatccaagagtctcagtgtctgtgctttattcagtaaaccaggagtgcgctcgagttttttagggcatccacgatgtagccgctcgcggggttgccctcgccggtggtcatctctgagagggggttcctgtccatgaccacgcagttggagtgcctggccctggacagcgccacgtacacgtggcccggtttaatgttcctgtggttgccgaagcagatggcgaccttgttcagggacagcccctgggccttggctatggtcatggccagcttggagctgatgccatagtctcggatgccgcagaggtttagagacttgtcctctattgtctcgtacagcttgttagtgttgtgttccaggcagcacacgaagccggcctcgtccttcaccatcagcctgggcatgcgcgacccgccgggctcctggggctgctgtcctccccggaggccgaagaagacgttgaggtgcgtgtatcccaggagcgtgtagttctcggtggtggaggcatagtccaggaggccgtggagacgaggctcatcggaggtgaattcgatgttgtcccggatcagcatgttgttggtgaaggtgcaaaatgggaggtccctgaactctcgcccaccgtagcggacggccacgtccaggcattgcctgaagtaagccttgaggtcattatatatgtttaacagggaggaaaggggggcggaatttgcggcagggggggccagcacggaggcgtagaaggcggcggcggggctccgctctccgtcccaggcgacctccagcggcagttcacccggctgcagccccgcggtcacctctggctcccacgtgcggcccggcaggggcacggccccgagcgccgccgcgtatccctcgtttttacagagagaatggccgccgtggctgaaggcgtagacccccccgtagatgagccgggccaggaagctgtagacgtactcgggctgctcgtgcccgtgggcctccacgaagctgtcggcctcgagcgtgtccatgaagtcgccgaaggtgccggtgtagccgcagacggacttcttggtcttgcagttgacagagacggagctgtgcttgacgtaggtgacgttgtaggtgaccttgaccctctcctcgtcctgctcggtccccacggggaccatgtcctggtcggcgaactgcgagtagttgccgaggcgcgcgtagttcttctggagccaggtgtgggccgtgaggcttggcaggccgaccagggtcttgtactgggcaaggggctcgaggaacacctcgcactccaccgggcatgtgaacatggtcacgccgccgtcggcgcccccggtccctcccccggcgcgccctctcgtggcggtcttgagggtggcgtggagggtggtgaggaaggttttgacttctgcgtgggagaggaagagccgcgtccagcccacgtactgggccgggtccatgatggccgccctggggaccacgaagcggtcgacgtaggccaggatttccggcgagagctcgaggccgtactcgagggtcttcatgaggtgcccgaactggacgtcggtgcagcgcttgttgttgatgaagagggcccagttgcgggccacgtccacgtaggccgaggcccgggggttgcccaccaggaaggtgaggatgttgtcgcactcgcggatcttgttgacctgggtctcgtggctgaaggaggactggaaggcgtctgtctgggtgggcgagcccacgcagacgatgcaggggacgtggccgctgcggtagaggggggtgtgcagccaggcattaaagaaccagtagcagaagaccacggcggtcaggatgtggacggagagggtcccggcctcgtccaccacgatcacgtttgtggtccagagctgcccctggtgcatgtcccgcagggcctcgaaggcggcgcccgagacgcccgagtagagccccctgggcttggtccgcctgaactcggcggcgatgtcggagagcacggtccagtacttggccaggtccctgcgctgcagctcctccagggaggcgtccgttgagcggccatgactgctgacccgcggcgtcatatttatgtggcggctcttgaacccgaaggcgctgtagacggtggggcagtaggcgcggagggtctgggagagattttgcgcggccacggttgtggcccccgttaccaggcagtccatcgtgtggtgaaggcagctcacgctggtgctcttgccggcccccgccgtccccgtgaccacataagccgagaagggcaggaaggggggctctgagagctccgggtcaaactcgggggagaaggcctccatatccgggagctgtcgggtgcggcgcctggccagggtccctatcctcttcactatccgcctcacggaggcgtccgaggtcatgttcagcatgaacgtggacgagagcgtcccggacgctctcggctcctcggccatccccgtgcaccccacgcccgcctcggtcaggctctttgagatcctgcagggcaagtacgcctacgtccagggccagaccatctacgccaacctacgtaacccgggagtcttctccaggcaggtgtttacccatttgtttaaacgagccatctctcattgcacgtacgatgacgtgctacatgactggaacaagttcgaggcctgcatccagaagcgatggccgagcgacgactcgtacgcgagccggtttcgcgagtccacctttgagtcgtggtccacgaccatgaagctgaccgtgcgtgacctgctgaccaccaacatctaccgggtgctacacagccgctccatcctgtcctacgagcgttacgtggactggatctgtgccaccggcatggtgccggccgtcaagaagcccatcacccaggagctgcacaccaggattaagagcctgcgggagcgctgcgtctgccgggaactgggacacgaaaggaccctgaggaatatcgggacggaattatatgaggcaacgagggaaataatagagtccctcaactcgacgtacatcccgcagtttacggaggtcaccgtcgagtacctgccccggagcgacgagtacgtggcctactacagggggcgccgcatcaggctgcacgtgctgttccccccagccatctttgccggagcagtgaccttcgacagcccggtgcagcgcctgtaccagaacatcttcatgtgctaccgcacgttggagcacgcgaagatgtgccagctcctgaacacagcgccgctcaaggccatcgtgggccatgggggcagggacatgtacaaggacatcctggcccacctggagcagaactcccagcgcaaggaccccaagaaggagctgctgaacctcctggtcaagctctcggagaacaagaccatcagtggggtcactgacgtggtggaggagtttatcacggacgcctccaacaacctggtggaccgcaaccggctctttggccagcccggggagacggccgcgcagggcctgaagaagaaggtctccagcacggtggtcaagtgcctcacggaccagatcaacgagcagtttgaccagattaacggcctggagaaggagcgggagctctacctgaagaagatccgctccatggagtctcagctgcaggcttccctggtggggacgggcgggcaggttgggcccgcttcagcctctgccgcgtcagccgccgagactgcatccgcggatatactgacgggcagcacctcctccgcgatcgagaagctgttcaactccccgtccgcgagtctgggcgccagggtgtcggggcagaacgaaagcgtcctcaacagtttcgtttcccaatacatccccccatctcgggaaatgactaaggacctgacggacctgtgggagagcgagctctttaacaccttcaagttgacacccgtggtcgataatcaggggcagcgtctctacgtcagatactcgtcagacacgatctctatattattgggtcccttcacctatctagtggcagagctttcaccggtggaactcgtaacagatgtctacgccacactgggcatcgttgagatcatagacgagctctaccggagcagtcgcctggccatctacatcgaggacctcggtcgaaaatactgccccgtgagctcgaccggaggagaggatggcgtccggccacaaccacccgcccggggagctccggagcctggccatgcaaaaagtaagcctgcgcgtgaccccgcgcctggtgctggaagttaaccgccacaacaccatctgcgtggccaccaacgtccccgagttctacaacgcccggggggacctcaacatccgcgacctccgctcccacgtgaaggcccggatgatctcgtcccagttctgcggctacgtcctcgtgagtctgctggactcggaggatcaagtagaccacctcaacatcttcccccacgtgttctctgagaggatggtgctgtacaagccctccaacgtgaacctgatggaaatgtgcgccctgctctcgatgatcgagaacgccaggagcccctcgctcgacctatgccgggaggtgctggggcgcctcaccctactgcactccaagtgcaacaacctggactcgctgtttctgtacaacggggcccggacgctgctgtccaccctggtgaagtaccacgacctggaggagggggccgccgaccccgggccgtggaacgagggcctgagcctgtttaagttgcacaaggagctgaggcgagccccctcggaggcccgtgacctcatgcagagtctctttttgacctcggggaagatggggcggctggccagctcacccaaggattactgcgcagatatgaataaggaagaagacggcgcatcgcgcttcacgttcaacctgttttatcaagattctttattaaccaagcatttccagtgccaaaccgtcctccagacactgagacgcaagtgtctcgggagcgaggcggttgcaaagattattccctagaataaactgagaaccgtcatcagtaaatccgtctctcgcgtgatttccaggggcatggcgtagccggggtgcagggcggacagcacgtccaacagaaaggccataatctcccgaaagtaggcggtggggctgagggcgtgctccgtggccgtctggcacggggccgggcgcggcccgtcccggtccaggagacacacgtggctccccgagagccgcagccgtccgctcctccgcaggcgctgcagccaggcacggggaagcgcccagaaccggtttcggcgccgcccgggggccagtctctgggtcagtccgcggaccaaggtcagcagagactcctgtgatggaggggccccacccgactcgggtttcgtcgccgccagccggtccagggcccaagcgaaggcctcatgccagaccagaaaggggccagaattctctccacgaccacccacgtagagatccccctgaaagatggccatcagcagacagcctggtccccgtgccttcacgcgcagtgggtggggccgctggtctcccgcccatccggccgccgcgtcctcgtcgacccgcatctcggcctcctccgcatcttgcacgcctttcaggaatgcctagagagtagacacgagggatgccattagccggaggttccacgccaccattccacccgttaacgggaagaatggcggcggggtgcctgcgccgagtcaccacccactcccccgctactcacccgcgccagggtctcctccaaaggatgggcggaggtcggcgccacgaagcccaggacaaactcgtcggcataggcccaggtcaggcccaggtcagaggccgcgcgcaggagggtccgcgtgacggccgtgtagaggccccccagggcccggcgggtgtcggcggtgccgtagtggtggagggcccgcagccaggtccgcacgtcccgcgcctgccctccggcctcaggcgtctccacaggggctccagcgaccggcaggtggcggagggccagctcgtagagccaccaggtggcgtccgcctccagggaggccgcggcctcggcgcgcccgaccaccgtctcgggcttcttccttccctcgccgccttcccgcacgcagacgtggctggggttaatctcccgcctggtcagggccagacctgctgcggaagccgctctggctgtctgtcgcaagcgtcggcgcccgggccgcgcgttgaggcccaggagcgagtcggccaggggggtgccggcaacgcccaagttcagatccaggagcatgaggccctcgctcctcggcgccaggtgccaaaatgccggccgcgcccttcccacgtagcggatgggcagaaaggggaagccgggaacgtagggctggaagtccgagcccccggggcagagatcggggtccaggaggtagaagacgggcttggggccgcggtgctctgcgtagcacgaggcgtagatgctccggaggaaggcgtagaggccgccccccgccaggccccaggagttgaccagccaggactcgaagcccccggcgggagtgaggatcttcaggctgacgcggtgctccgaggacgcggacccatcggaagaggccagatccacctcgtagatggcggggcgcaggatgtctcggaagggcaccggcgagggggtgttcggggcctcggtggaataggtgaagacgtccccgcgcggcgtacggatgtagacgtccagctgatccggcgcctctgccccccgggcgcaagagcgggctccgggtcgcacccccgctcccgcctcctccgcatcatctgtctcaaagccccctgagccgctgggcggtggttgttgctgacatcgcccatcttcccccggggagcagccctgacaaaccacctgcgccgggaagaggggctggaagatgaggccgtgttcctcctccacccggacggtggacaggagtctgcggtcccgggttcgaaaagtcaggtcaaaaagatccttctcggcggcgtccgcgaggggggcaataagccccccgctcctcctgagctcccgttcccagcgcagaaagttcagctccgtggcagtgggggcgttcacgcacgggggctccatggagcctcctagcgggggctggcaggcctgcaccgcgatcagagtctccacatcctccccccggacgggcacgatgcccacaacccatataccccaccaccgccctccgacctgggtaacattataaaaggtaaccgagctgacccgcgccctgacactctccacgggtgtttccatctttcacagccgaggacgaggaccggcctttttaaacatccggtcacaccctttgcaaattatttaaaaggcgaatgctcatcccggaccatcgcaatcatgaagtcctccaagaatgacacgttcgtctacaagatatgggtgaaaacgctcgtggtgtactttgtgatgtttgtcatgtcggccgtggtccccatcaccgccatgttccccgacctggggtacccctgctacttcaacgcgctggtggactatggggccctgaacctgacccagtacaacacggcccaccacctcacccccatgctctatctggagcctccagagatgtttgtctacatcacgctggtcttcatcgtggactgcgtggctttcatctactacgcctgtggggaggtggcactgatcaaggcccgccggaaggtctcgggtctcacggacctgtcggcctgggtctcggccgtggggtccccgaccgtgctgttcttggccatcctcaagctctggtccatccaggtcttcatccaggtcctgtcctacaagcacgtcttcctctcggccttcgtctactttctgcactttctggcctcggtcctgcatgcctgcgcctgcgtgacccgcttctcgccggtctgggtggtcaaggctcaggacaactccatcccccaggacaccttcctgtggcaggtggtcttttacctgaagccggtggtctccaacctttacctgggctgcctggcccttgaaacgctggtcttctccctcagcgtgttcctggccctgggcaacagcttctactttatggtgggggacatggtgctgggggcagtcaacctgttcctcatcctgcccatcttctggtacgtgttgacggaggtgtggctggcctccttcctgaggcacaactttggcttctactgcggcatgttcatcgcctccatcatcctcattctgcccctggttaggtacgaggcggtcttcgtctcggccaagctgcacaccacggtcgccatcaacgtggccatcatccccatcctgtgctcggtggccctgctcgtcaggatatgtcggatcttcaaaaccatgcgccagggcactgactacgtccccatctccgagacggtggagctggagttggagccaacgacgaggcccagaacgaggtcctcgtctttacgcggtggcaacggccgccgttcgtccacctcctccagctcctccaggtcatcgtccaagagacagaggacggtctccgcccaagcccacgtcccctccgttttacctatgacatcagacagcgaggatgaaatctttccataattcaataaagtttgaaactcaaaggttgcgttaccgtcatgctttcggggtaacgaggggaggcggaaattggagcagggttggggaaatgacaatggggtccctagcacggaaattcagagttgggaaaaggcgcccaggtctggagctgaaggttttgggctccctatcagcggctctgccctgccccctggctaggggctccgggctctggctagaggctctggctcccttgctaggggctctgggctgtctcccggctagggactggaacttgctgtcagtgggttgttgtgtgggggcaagcaccagtggccaacatactccgggttccatgtgtgttcccactgccatccacccagccttttttgcagggcctaacatctcccccctttattaacacctcccgttttcccctttcaccacaggaaacaaaagtcagtttgatcgtttatttggcagaaatggcagttcatcattctcagactcatcatcgtctagcccctcgttggtctccccgccagccctcccttcctcaactgccatcagctgagcagtctccatactgaaggcctcgtagtcttcatagaggttaatcatgcccccatccacgttggctatggagttgactcgtcgccggcaaagagaccagagggcacccatggcgcggtgtcaaaagtgttgtctgcgtaggctttccaggagccggccgcggcgttcagggtctcgcggatgacagagtcaggcagcaccacgggagtcaccagcaccgccacggggatctccacagaggcgtccagcagcaggtccgagccgagcgtgcaggtcggggggtctaggggtgagcgctttcggaagaaggcagttacaatgttcacccggggctggcagtctctgtcagccgcgcctcccccgctgtgccggacgtagtctccgacaatcttgtactggaggagcacctgatagaagtagttgtgccgcggattgatgaagatattgacggggacccggtctttaataccaatgcgcccagcattttcgcttgggtccgtcatgacgtagagcatagactccacccccctgttggcagccagggcagccgccacctggtcgtggccgggccccagtttgcgcttacggacctccttgaggttccaggcttcatcctgcgtcagcaggtagtcgccctccgagggcagccgcccatccgggacatactccacggtgggccgggctatggaattgatgaagcggataaatgacctcttgcagggcctctcatacagcgccgtgtaggaggggtagatggggtcaaactcagacttggagaacaagtacttgaagcggcactttatctcgtagatgcagctccggtctgtgaacacgataaagtccccccgtgactccacgttgacgcacaggtccagagacacaccaaaaatgccgtccgtcgggcttatcatgaatccaaactggcggttggcggccgcgtccccgcagatgagcttacagacaatgtccttgaccgtgtcctcgcaccggaggccaaacgccacgggccccccaaagtaatgattggtggagatgggagcgggctcaaacaccttggtgggaccattcttaatggtggagagcagctttgatgaggaaattatgccatttcgcaatatgtcccacatcaggttctcagactgcccccgtgtcatggactccacataagagcagagcaccgtcttttgctcgtcagtggcctcctgtaggccccgatagatggatattagggagggactttcctggctgtcattctcttggattaacgaggagacaaagtcacagaagccagtttcaccagaaaactcctgtatttgcttacagagacaatagagatagacgtagcgcatggcgggcatctggggcgggcggtcgaggtctcggacaaagtcctcgatctccggactgcggaggaagcgggcgaaggtgtaggaggtcatttcctccatggggtcctcgagttcatcaacgtctgccatcgggaccaaagtactcgtctgccaagagctccgccaccaaacctgggagaagagggtgctcaaaacgcgggcgacagttgaagaagtggctctccttgaacctctttttaaggccacggcaccactgcaagaattgactcatgtgctcctccgtgacatccacgcacggactctcgccgcaggaagtcaggcccacatccaggttcaggttccacatctgcgacagcacctccagcagcaccaccttcggggccgcaaattgcaaaaagtagagcgggtccgatcggtcaaagcccatgtcggggttgggatagggaatcttgtgggtggtgtcggccagcttcatgacgccgtagagcagcgagtagccgagcgactgcagatccagtcgcagggccgtctgcgcccccacggggccgcaagccgagggttcagggaggtgccccgcccccctcaggatatagcacttgctcaagaggcagagggacttgtaggtgtccttggctatagaaaagggctccctctggtagtaaaggcgatacagctgccggcccttggcggactttagcctcacgtccaggatcttgttgcggtcgtggagggaggccgtgccaaaatccgtcagcaccagcctccccatgccccacatggtgggggtgaaatccaccaggatgttgctggggctgatgtctgagtggaaaaggccgcagtgctggttgagaaagtaaacggcgtccttgaggccttgaaagccctgcaccaggggctcgatgctgctgtcattccagtggctataatcctggagactgcatctgaactggggcataaagagggcgtgacaggatgtgcaggccgacaggtagtccaccagagccttgtcctgcccatcctcggacgtggcctttccaatctgaatcatgtcacagaccatcagctcgtggtatagctctgtcacggcgtcatagagcttgacggtggcgttctccccgtacacatacacggccccgtagctcccccggcccaacagatactcgcaggtgatggggaggtggtcacagcgcgtcagctcctccggcagcttcacgtatagcgtctccgtcatgtcatcaatgttggtcaccttcaggtgcttgtgctgaaaggtgaagtaatcaatgacagtcaccttccccaaaaaggcctggggctcccgagggggctctggggagacactcaactctccactgctcaaggagttcgtcggactcctctccgcgtccatagtcacatcgtccatggtcctcaaagggctcgagcgcctggcgcagctcgtctctggcctcgagttcctgctcacgaagctcctctacccgttccagctgcttgtagtttatcttggggaaatgggtcttggttgaggtgactaccctcctgtaggtggaaatcagctgcttggactcaaacgtctcctgcgcacgtcgcagggtctctagggccacacgagcagatgaaaactgcgtttcaaagagagcgtgttcactcccaaatctgtcacgtgcgctcaccgccgctctcttttctaccgaggctctgagttgctgggccaccaggtctcgcttagcactactcatcttcataagtcaccatgtccgccactatggaacccagatcataggtgggatagaggacagtggttccggtagcagcttcccggtaattgcccacggcgtccaccagatatctctcagcctctcccgttagaattaggcagggatcatacgtgtccaccggtcttttatactgagcgtttaggttttgcttatgcagcagacacaggaggcacactcgagtgatgcaaacgggttcctgtggtagcaggctgagctgttttgccattttattcaggcggctcacgtcaaagggaggagctatttcctccccctgccagtcacacacatccgcatagagggcatacacacacctggtgaggtgtgccaggaaggcctctatgttggctcgcggcgtgtagatcgcagtaggcagcagaatagggcccctcctgccccgcgccgccgagtagacgcagtgccgctcctcgcagtgggccctgggaccgtagaagagtgcccataaccagggaagcgggtcttcaggcaccagagaggtccaggtttgggagtgggccaatatttgcaaggcctgacctataacctcatctttgttccaggtcagcgcaattcgcatcaggtccccgtcgtaggcctcaaaacacagacccgagcccttctgcacctgaggcggctctgcgggtgttaaccccccctgttcaccgaaccgccattcttccggtaaacgagggttgaggggtgagagctccaaggccaggctcgagaagtcatagtcatccccggcctgctgacacgggtcccgggcctcctgccagggcctggtgtcctgggggagtatattggtcaccaacagaaagctcttgagcggcgtctccaccagtttgaattgctcgggtgtgtcctgacaggcctcaagggccagttccagacactgcccatacctgcgggcgagcattgggtcatctggcatatcggccttgaccgcgttaaacatgctgtacgcctcgcagcgtggccggttggccgagaacctcagaaatgcccttcagcaggacagcgccacccaaggctgtctcggcgccgagaccccaagtatcatgtgcacgggggccaagtcagacaggtgggcgcatcccctggtgggcaccatccactccagtaatttatattgcccaatgctgagagcttactgccgccagtacggccccaggcccgtgtttgtaggctctgatgcaacattacccatgttcggagcgagccacgcccctcccgccccggcccaggcgcagatgtgcctcctgccggagctccgcgacaccttgcaactcctgctgcccccacccagcctggcagactccgaggccctgactgaattcaagaccagcgtgtcctccacccgtgccatcctcgaggaccccaggtttgtggagatgagggagtttgtcaccagcctggctggctttctggggggacagtacaagcacaagccggcccgcctagaagcattccagaaacaagtagtgttgcattcattttattttctgatctcaattaaatcattagagattacagacaccatgtttgacatcttccaaagtgcttttgggttggaggagatgacgctggagaagctgcacattttcaagcagaaagccagcgtgtttctcattccccggcgccacggcaagacctggatagtcgtggccatcatcagcctcatcatctcaaacctctccaacgtgcagataggctacgtcgcccaccagaaacacgtcgcctccgccgtcttcactgaaatcatcgacaccctggcaaagaactttgatgccaagcgcgtggaagtcaacaaggagaccagcaccatcaccttcaggcacagcgggaaaatctccagcaccgtcatgtgtgccacctgcttcaataagaatgtaagacctgacgtttcagtacttggcagttgtagagcattacccggctgtaaaagtcagaaaagcgtagcagggtccagggctgcggcgtgcacgggacctctttcccattagccagaactccctgcagaacacgagacatatccgggtgcatttttggtgggctgaatctcagtcccactacaaagggggcgtcctctggtttaaacatgagccccaggagggccctgtgcccagttaccggcacataatcattgtttagggcagagcagggcagcagacagggacagataccggaggtgccagggctgtcatctgccgtccagccactcaggacgttcacatgccccccggatccataacaagtcacacagtccccattctcacatcgcgtgagcaggttgataaaatgggtcagagatgggaatgttggcatattggggcagcacattaacatgtccatgttgatgaaaaacatgtacagggccccttctgcgtaccaagccccgcctcgcccaagtggagtaatctccgagggtgtgagatcttgcagttcttctactgttttaacggcggttgaagtggcgaaaacatgggaggtcgtcagctcctgggagctcagtattgggttgcccctaacctccacaggcacagcctcaccctccgcatctgaaaacaccccaaagtacataagagtcaggttgtgtggcccctggacggacttggtgaagagaacctccggcctggccacggcggccagggttccattgatgtacaccgtcacatatgtgggcttcttcttcggcttgagcacgatgagggtgacgttcatgtgggtccgcggggctccggctatcctgggcatgtacacggccgacacggctgtagtggccgtgtaaaccttcatctggttcgtggaggcgtcgctcggcacccataggcattccttgttaaggaacttgcgcagcagctcccgctcattgcctgcggcggacgccatttcgggccagaacgtatcccctctcctcgggggcgagttccaattggcctttaataacaaagcccccgggcagcaccaaaaacacctgtccgtctgacgtggcggccaggtggacgcagtgcccgtccgtaccaaggtccactagcccggaggccgcttcagccagcccacccggggttccggagtcgataaccttaccccaggccgaggccccgtcctcgtacagcgggtggctatctacccagaggcgagctgacggcacccccggggagccggagatagctttcacccaacaactttcccagctgacccgtgtctcaatagtgaagaacgcttccctgagcagggttgattttttatagatactggggtaggaggtgggaataacaactgggatttcttgtttggctgtccagtcctgtgcagccagtttttccctgaggctggcagaaattctgagggccatagaaatgaggaagcgaaactccctctcaggagctcccaaaattgaaaccttagcaagatttgtggtcagcgaggtgtgggtgacagctgtcatcctgtgcagtctggcctgggcactcagatctggatatgtgacaacatagagagaatgggggctgaaaatgtgcaccgtcccgtggaccagggccctggagcccctgatggcccgccgggtctttgagaaggttaggggtacccgagggcgcgcccccgtggagacctctgccccgggagtcccggcaccagccccactgcccccaccagcgcccctaatgttgctagggttttctagagcagggtgatgtttgggcacagaggcctcggccgctgccttggtttcggtcccggcccttagtctgaggcccagacagagggctggactggcggcggtccccaacccgtgcccccctctctccacctcctgcgttatttccaatagaatgtgcttcaggtcgtagctgggagggatctcggccccggccgcggccgctgcagccggctcttcctcagcgtggccctcggggtggggaatagacagcgagggcgtcccccggacaccggaggcctccctgatcggacggccgtcggtcagggcccgtttggccccctcaaaaagagacaggtagtagagctggagctcccccacgagtccaccactgtcgtgaaacctgagggcggccacgtggaagggattgtagagctccggtcctccctcctcgcagtacacgggcacgctggtgaaggtgcctcgccggctaggccacccgggcaggacgccgcgcgccgcaaacacgcgacaggcccccgtgagaccagcccgatcgctgaagagggtctgacagcagaccacctcgcagtccggtacccgatcggggcagagctccgccatgaccgtgtctggcacaaaaatgtggacgaggaggggggttcccaggccgctcagcacctggttgtcaacgtggacatccatagctctctcatgcgcttggctacagagtcgtagcgcttgtttcttgtcgatttaaataacagggccccgtagacggtcttttgtgagtaaatggagatgatgacatggatgtagaggctgaggaccacatccaccgccttatcggagtaggcccctcgaaacagtgacaggcaacagggaaacacgaaggagactaaggccgggatgtggggcctcagggctccgtcctgatcaaagatggcctcgctgacccccgggataacattctcgttcaaaaagtagtgatagaggtggttgacaacggtccggaccaggccctggacctggtccggctcccacttttccctttgctcctgcgtgtcttggcggatctctgtccagggcctcagcgccggttggagatgcggccccatgcagttgcccgtgagggcacacaccaccccctcgtgggtctcgatgagggtgcactcgggggagccatcgcagacgtgatactctccgcagccccagcaggcaaacacggaggccatgctctcaggtaacgggaaatgagactccagcttactgtacgagcataggtggcgagaattgggcccagtgggagcaccctcgccgtcgggggagctggccccccggcctctcaccgctccccgcggcctgccgctcactcggccatggtctgacattccaccccggccaggtccaggagggtgcagatgttctccaggcgctgcacctcggagacctcctgttcaaagaggctgcccaccgcagagtagacactcgccacggtccgggtcaggtcggtggggtcccagctcagcagctccgcaaactcccgctccccgatagtaactcgcttcttctccttctgtgtttcagagcatccggggccagacattccacctgctgtttgtggacgaggccaactttatcaagaaggaggccctgccggctatcctgggctttatgcttcagaaggacgccaagattatcttcatctcgtccgtcaattcggccgatcaggccaccagcttcctttataagctgaaggacgcgcaggagcggctgctgaacgtggtaagttatgtgtgtcaggagcatcggcaagattttgacatgcaggacagcatggtctcgtgcccctgctttcgcctccacatcccgtcttacatcaccatggatagcaacatccgagcgaccaccaacctctttctggacggggcctttagcacggagctgatgggcgacacctcctccctgagtcacgggagcctgagccgcaccgtgcgagacgacgccctcaatcagctggagctctgccgggtcgacaccctcaacccccaggtggccgggcggctggcctcctccctctacgtgtacgtggacccggcctacaccaacaacacctccgcctccggcaccggcattgccgccgtgactcacgacagaactgccccggatagggtcattgtcctgggcctggagcactttttcctcaaggacctgacgggggacgccgccctccagatcgccacctgcgccgtggccctcatctcctccatcgccacgctgcacccccacctggaggatgtgaagatagcagtggagggcaacagcagtcaggactcagccgtggccattgcctccatcatcggggagtcctgccccctgccctgcgccttcgtgcacaccaaggacaagacatccagcctgcagtggcccatgtacctcctgaccaacgaaaaggccaaggcctttgagaggctcatctatgccgtcaacaccgccagcctctcggccagccaggtcaccgtgtccaacaccatccagctctccttcgaccccatcctatacctcatctctcagatccgggccatcaagccaatccccctgcgcgacgggacgtacacctacacgggcaagcagcgcaacctatccgacgacgtgctggtggcccttgtcatggcccattttctcgcaacaacacagagacactcgttcaagaaagttaattaaaatttattgatcacacaaatcccttgtaaagagacggatcacacaaatcccttgtaaagagacgggtccctcggggcagtgctggtgagcagggcttcgttcagggacgcgttcttggtggccatcattataccggccgcaaagatcagaatcagcatcagaaaagtgaggcccacaaacaccagggtccagagagggagaccagtcagcgaagacggctgtttctcgtcaggtggggtgggcagctcagcggtggtttcttcggtggcgtttgcagtgacattgcttggctcgggggtggaggtggtgctggtggtggcctcggtgctgacattggaggtagcgttggcggcatgtgtggtggcattggtggtgctggtggcctgggtagtattgttggacagggtggcggcctgggtggttgcagttgttgatatggtggtagcattagaggtggcattggttccagtagtggggggctgggttgatgcccccgtagatgtattggcgctggtctgtgcagtggcattggttgtactttccccggttgttggtgtggttccattagaagttgtggacgccgcaggggctgccgtgcctgtggatgtcaccagtgttgtgtttgcgccccctccggccgtggtggtcactgggcttgtggtcgttgtctgggttgctgtctggtttgtgactgccactgcagatgatgtgcctgtggcatttgcaggcgctggtgtagacgaggtccatgccaggctggtctcacagatgagcatcacagccagaacggctcctgctttgcctccggtgattgccatcgtgtctgtcagcagaaggccaccgggccccttttatagcccttgtccacgcccccggtgtgcgctactgaacaaccacatacatcacctcctccaagtatgcaggaaatgagcggtccgagagccggtcaatgtgacattgctttggctgtgtgcgcttggtgtccctctgctgcatcttaactttaccggccggggccaatgtcccggtgcagttagtcccagttgagctgccgccacttagaacatctcggaagtttggctttggaattgagccaaagtctgggctcgtcctatcgaagagataatacgtctcgtagtggcagtggtgatgcagcaccatggcggcgccggccagcgccctctgcatgtcctggacaaggcgccgttccacccggctgtcggcaccggtaatgttgaatgtccttttcctgtttgtctctgcctggaggttggagcacccggcctcgaaaagcgtggcaacatgcagggcggctgcctcaaatctcccacgccccccgaggctatccgtgttgttggggaaataagtaaccccgttgatgaccacggccgggatgagagtgtagacttcgcagaagggattggccggacacccataagacaggggcacgccaaacttgtggcccttttcctgtagcaccttggcccccaccgggacaaaagtgggaaggaaaagggggttgaagccgaagctgatcagccccagtagcgtcaagtagcccaagtagtagatgaacacgaagctcatcataaaccagcgcggtggctcggtcagggtctcggcgtctccatcacacacgtcttctttgaatctcatcttcccaccgcttgtcacctcgctcttcatccaggagggggccatggccgccattctaccagttaacgaggagagagagaggaagtccgcggaagttggcgctccatgctgaccctcgggagaggccgggtcgccagtctctccgtcgcctgccccggcgctggatgttgtctcgcccccgttgcgctgatggtcgacgagaggtggctcgggcccgggcccggcctccggcccggggacggcggcggcagccgggacggtgaagtcgtggaaggtagcggcccgtcccgtccggggctgcggggtcggagacttgtaaaacacttccaccctctccccgctggccaggacgcgccgctcgtggactacgccgtcttcctcccgggtgatggtatggctgacgatgccgcactccaccgccacctcctcctcggccatggcccccttctatcttaaccagcaggtggccgtcagggtctcttgagagtatgctgccgtggccaggcggggacccaaattaaatagtgacgctaaagactgtaggtaggtcatcataacacgcatgcgtgataaatcatccgccacagacagggcatccaggtctatccgggcaatctcatccggcaccatgtcctggaagagattcaaaaggtcgtggtgttcatgtcggatgaggctccggaccaggcgcatgctggccctgggcagcagggtcaccatgatgcaaaagtagaggctcaggttgtccagcagggccagccccagggggcccggcacctcagggagggccagctcgtagtggtgccccagataggaaacagagccaagatgcatgcgcacctcaagcatatccgcatccccaggcgcctgcatcaccaccctggagtagacgttaaacaggagaatcttctgtaggacctcctccgcgatgggcgtgggcagcaccatggggaaaacaatgtccacgtcattggactctagcttaatggtggcgtgctctctgccaaataccgggggcatgacgcttaggctcccggtcccgtgccactggaagaagggctggtatttgttcttgatggcgtaggtctggcccgggacaatcttggtcagcatcaggctgtccacgctcacttcgtccagcacggccagggtgcaatcggacaggtagttgtacatggacacgtagtccggcacggtctccagagagtatacctgacccagacccaggccctgcacgttctgcgttccgtgcgtggatgccaggggcagaacgcagcctatcctctgctgcagcttggctatctcatcggtatagagacgagaggacagagacactaccactttcaaatccatctttattgacaattatcaaaaaaccaccttatttccaaactttaatattcttcgcaccggcgccacctcttcaattatatagtgtccgtaatggatgggggcgtgtgtctgtttgaccgacataaactcatcgatgagtgccctggaggaggcagagagggcggggaacgcctcctggaggaagctgcatggctgctccaggaacacgtcagtgcctgcaatcaccacaaactgcacctcggtgttgctggtggccgggtgcccacccaggcgctggctgtactcattgaccatgttatagaggcccctgttgttgcgcagcagctcctccttgttgaaaaacgcccggcagggactgtaaaggcctgggacagccgtctggcggtaggaggagttgtacatgatgtcacccagggagcccagctgagaggcccagggattcaccgtgctacggtactcgtaggcggcatccgggcgggagtggtcatagataagcccctcggcggcctcctggttgtagttctcgcaggaaactacacaggcagcgcgtcccctcggggagttggacttttgaaaataggccacgtccgccgtcaccggggtaactataatctcacaggtggcttgctgcccatgacagagtcctggagctccattgacatttgtcatgccagccaggtacgtcctggggtcccggagcagcgtgccattgcgctgagctcccaccttagccttaatgtagtcgttgacctgctgattggcaaatgcctcggccgggaagacgctgaagaagtcctgcgtgtggatgcccatgtcagtagtgatggccgccacccgggcgggggtcatggtggagctgtagctgagaccggtgtcgatggaggccatctcgtgatgcacctcaaaggtgacagcgtccaccctggcctcccggcggctgacattgggggtcccgataaacatggaggtggaggccctggagctgaacaggatgttctccgagaggatctcgtccgtcctgaccaccgtcatggccaccccggggtgaatcttgagcttggcctgggcgatataggccatgggggacatcttgatgtgcatggcagtcattccactgattgaaacgagggaaggaagacattcagccgcgtattttgccatgggagagcggtgccactcccggtactcagcaaagagctcctccggccggttaaaggcctccacggcccgctgctggggatggcgcatgacaaatgtggcgacgtcctggtgcatggtggcggccacccgagggtccccgtaaaacaagtggaaggggacagcgtggaagagacactgggtgacggccctggtccgctcagagaaggcaaatcccaccagcccattcaccaggacagtctgctccgtccgcttgtcggcgggattcggggccagctgctgggtaatgtcgttgtccacagacacgcgcaccgcacgggtgaatgtggggcaggtcaggaaggaggcacagaggtccctgatcatgcccacggtggggcggagatcggagatctcaaggaggtccctgagcgtgccattctccaggttatcgaggatatcctcgtccctggcaaagtggtggctgaaaaccggcccattgtaggccagcgtctgggccacgttctgaaagtccacccccaggccgcacatgtgggcgttggtgcaggccggtaggaagacgtagtaaaagatcttctccaggacgtccgcgtgcccctcatccacgtaggggccaaggtgcagcctgaaatcatggtcgtggtctccgttaacgcggtaaccatacatggccacaaactgggcggccatctcgtccagatttcccacgttctcaataaactgggctgtggccaagggatccgcatagacctcattcccaatgaatatctggggggcccggtcgctgacgttaaggagatgggtgaagatgtcagtgtaggccacgggggggagcaggttagggtccaagagcgcgcaaacgtactgacccacgctctcgtcctccaccacatccggcccggccaggcgcagcagtgcctgctccagggctatcagttccccataaattttccgatacatggaataggcctccttggtgatggcattgttgcccatgtgacggcagatgaacttgatcatggagaagctgttcacaaaagccagccgccccgcccgctcccagtaggtgttgatgcacagggccaccagaggtacattcataacaaacttctcctcgaatccgtgaatcatggactcgactacataaaagaaggctggataagcagtgtcataggcagtgtcctgcaccgtgtcaatcacagcctggtccaccacatgtgccagagaggtggcagtctcaaactgctgcccccgggcttcctggaaagcggccggggccaggggggtcggcaggttccccaccatcagtcggtgcacggcccggtgccgcgccctttccccggcatccctgccaatgtagatatcaaacagtggatgcagctccaagcgcaggaggtcataattggacgggtggaggaaatcctcggtgggcattccgcacttgagggcaatatccgtgacgggggctgcatacttgttgtcatagaactcatccacaaccaccagcagattcatatgatttggcctcctggaggccagggaataggtctcacgcctctccctcggggcctgggccgcattcaggctgttcagggtatgggccggcgtgtggagtcgggggtgacagagaaccttgagggcattctgtaagttaaacgccaggagaaggttgttcttgttcacgatccaggcctccaccggtagctgctgcgtgggattctccagcattttgatggcagccgaggttgtgtacttgggattgggcataaacaggcctactgggaaatagtagctgtattgcattctcctgtttagcgggtagggggactgagtgtcattatacatcttctgcaggctctccacggccaccgcgttgctccccaacttgatgacagcggctgagatgggcacccgaggctggtcatcgacccccgcggccacggccggcaggtccgcctgcgcactaccggccttctccggcgagtccacgatcctcgccatgaaattctcaaaggtgcgcatcacacggccgtagctcacggccgtgaccagattctccccccgaaccacaaaggaggcgtagctggagggccccatgatctggttgtcagcctcttcacccaggaaggtcaggagctggcgcagaacgttatctgtgactatgaacactcccccaacagggtctccccccttggcggtggtataggtgctaacccccttgagcacattctccccggacacggcagcgaccatctcggagaggcggctgcgcacgtactgggggccagagcccatgttctcggcccggtccaggaagaaggagtgctccagcaaatggcgcttgaacatggcaatcaggtcagacttgacagtcttggagagccccctctcagtgaaggtgggatcagccaaggtctgcaggataaacatgggcggagcatggcgcagcttcacgctcaggaccgtgttgatgaggcccctctccagggcgtcgaccccaaactggagggcagaggccacggtcttgacggcccccacgtactcggcgtactcgaccggggtctcggggatgctgtgcagaatctccagatccagcatggagagctcaatttccgtactgatgtgatgtttgtggcaattcttgaccacgatgaaggtccgctgcttgctgggtctccggccgtctccatgagcaatggtggggacggaaatgcgaaattgaatcttgccatccgtcatgcgactcaggtctttgaattccgtgtttacgcaggacacggccagagccgtctccaggaatcgaacgtactggatggcgttggtgtagaccccgagcagcacctcgaacttgatgcccgcctccctggcatccttgcccacaagcagatcgaagctatgaaacagcccctccgccgcggactgcctcaggttcgagagcaggttggcgtccaccgtcaagtaggggaagggtctgttctccacaccctcgtttgaggccatgtctcacggtaaggaggagctggggggaggaagcgtcaccgggcttaagggcttctcttcacagctgggtctgttttacgcactggcctgcaaccgcagcccacctccggcgcttcccgaggaggccgccctcctgatcaagtggctggactcggctctgggcagagaggccaccttttacgcgtgtcgggctatgcggcggcttttgctcggcgttatccgaatgaatgacggccgggagccgccacccggtttaataattctgagtcccggcaccggccctggccccctgggagtccagagcctggagcacacggacgccgaagtgtggtacgctggcccctctggctcccccgggcccctcccggcggcgcccagggtcatcacgtacggcggctgccccgactccgtcaacacgagcttccatgtttcgcctgatcatccggtacctgtcacaccaccagttcgagcgctgctacgagcagttctgccgcgtggtcccacgtcggtttactagccacctgcggtcgcaattttgccgggatgctggctcatctgaagcgggtgaccaagatcccgccctatccctccttcgtcgggcgggaagacagagtcaactttcacctcttctcctggagcacgttcatactgtcctggcccaacaacgccaccctccgggagatcaggacaagggccatcaccaacctcacccgccaccccggcctcgtggatgccctgtaccacgccagcccgcagacccccttccacacccacagcggcgccctctgccgcttcgtcacctgctgcaactgcaccctgcccaacatctcaatccggcagcgcaaggcgggggaccggccagaggacctggagatcattctacaggataacagcgggggaaggccagcgagctttcagttcccattctccccccgtggcgccctcttgcggtcagtgttggcagcggcgctgctgccggaggtctcggtggggggtcaggagacgcctcggagacacatcttgggcacggaggcccaggccgcacccgcggccagtccgggcacagaccccgcgcggagactggtggcgctgctgcgccgggaagacggagcccccaaggatccccccctgggaccgtacggacgcccgtgtgcccacaccaccgaggagagcgaggacgaggacggcccccttcccgacgccccgtcgcccccgctcaacgccggctttcaggcctgccggtcggtgcctgtggggccggggtttcgtctgctcgtgttcaacaccaaccgggtaatcaacaccaagctggtgtgctcggagcccctggtcaagatgcgggtctgcaacatcccccggctcatcaacaactttgtggcccgcaagtacgtggtgaaggagacggcgttcaccgtcagtctgttcttcaccgacggggtgggggccaacctggccatcaacgtcaacataagcggcacctatctgagctttctattggccatgacgtcgctgcgctgcttcctgcccgtggaggctatttatcccgccgcggtgtcgaactggaactcgacgctggatctccacgggctggaaaaccagagcctggtcagagagaacaggagcggggtcttctggaccaccaacttcccctcggtcgtgtcctgccaggacggcctgaacgtgtcctggttcaaggcagccaccgccaccatctcgcgggtgcacgggcggacgctggagcagcacctgatccgggagatcacccccatcgtgacgcatcgagacgcgaaaatctcccggatcaaaaaccggctctttaccctgctcgagctccgcaaccggagtcagatccaggtgccgcacaagcgcttcctggagggcctgctggactgcgcctccctcctgcgcctcgaccccggctgcatcaaccggatcgcctccgacggcctctttgatttctccaagaggaccatcgcccactccaagaaccgtcacgagtgcgcgctgctgggccacaggttatcggctaacgtgaccaagctggtggtgaacgagcgcaagacccgcctggacatactgggccgtaatgctaactttttaacgaggtgcaagcatcagggcgatcgacgacagccacccatcttcctgcccctcctgagacacatcagccggcgtctgggcctgggccgcgcgcccctgaaacgagaaatcaaccagctcctggcccgactgcaaagaaagaaagccccgacccgccatcggaatgctcaagtgtaagcagccgggcgcccgcttcgttcacggggccgtgcacctgccctctggacagattgtgttccacaccatccacagccccaccctggcctcagcgctgggcctcagtggtgaaaacgtgcctatcccagccctatttcgcgcctcgggcctcaacgtccgggagagtctgcccatgaccaacatcagggccccgatcatctcgctggctcgtctcatcctggctcccaatccctatattttggaggggcagctgacggtgggcatgacgcaggacaacggcatccccgtgctcttttcccagcctatcatcgaggtgaaaagtgggcctgagtctcacattaaagccccctcacagctcgtcatcgcggaagactcctgcctggaccagatcgcccccttctccgcctcagagcatccagccttctccatggtcgagtcagtgaaacgtgtccgggtcgatgagggggccaacacccgccgtaccatccgggaactgctggaaattcccgtgaccgtgctctcttctctcaatctgtcccccaccaagtccattttgaagaagccgcctgagccacagccggagcctcagccctccttcgatccagccccctacgctcgcgtcttctacgacatcgggcgtcaggtccccaagctgggcaacgccccagcctcccaggtcagcaacgtgctcatcgccaatcgctcccacaactccctgaggctggtccccgacacaaacctccttccccttcagcacctgtttctcaagcacgtggtgctcaaaagcctgaatctggagaacatagtggaggactttgaagccatcttcacctcccaggacgacaaggtgagcgaggcggagaccggggccttcgtgaagctggtggagacggccaagaccaccatagagaacatagtcttctgcctcaacagcatctgctctgcctcctcactcccagacgtagtccccgatgtcaataacccaaacatcagcctggctctggagaagtactttctcatgtttcctccctccggcaccatcatgagaaacgtcagatttgccacccccatcgtctggctcttgtgccagggggccgagctcagcaccatggctcaattccttggaaagtacatcaaaatcaagaaggagaccggaatgtacacactggtcaagctttattacctgctgcgcatctagaagaagtacatgacaattttgtgaaccagaaagatccccagggcaaatgcaataaagtacaggatgacagccaaaattacgtgtgctctttcttcgtagaggccagcaatctccactaccgtcccattggtagtcaggagcagatagtggacgtggaggttgttaaaatcaaaatagttggaggtaaagatggagttctgtacttcctgggaggtaatgtaggtcatagtctgcagcccctccttctcatcgtaactgagcaagggcaaaagccgcaagaagacacactccttctgcgttctggtgaagttatggatcttggggatctggcggggctccccggcgacagagtctcgcgaacatttattcattataacggggaagagaaagagagagctgctgagataggtcgtgctggcttcgtacagtgccgagcctcgcacctcccggttgctggagacaatgaacgtcacgttgataaggggaatgatcatgaggaccttgtcgagcctgtccacgcagcgggaggcggggaggtgccacacatccctgtcctccctctccagagagaggcgcccaaggaacccgtccacggccttggaaacagacgccgggtccagggacgcgtcctgcggggccatgtttagcagcttttctcgcgtaaggtcaaatcgcaggctcaggtagcacggcgagaagaggccgctctccgtccccagggccagcccctccaagacctccccaatctcgagggccgagcacagggcggtggacagcagctggtacagggcgaggttgggcccctgcgtggtcacgttcagcctcagctcccgtagcaccacgtggctgccgatgaacagggtctccctcatcacggtgtgcagctgctggaagagggggtgacggttgtaggccgcgaggaggaccgaggcggcgcccccgacactgccgctgtagtccccggccttggggtagccgacggtgagcagtctcagcgcgtattcctggttctctgtcgtcaggtgccccagctcctccatcctaacggtggtcatcagcatggcggccagacgctccaacccgtaggacctcatgcccttgacggtggccccgtagcacgtgccgaggatgtcttccaggacaacaagctcaaagaagctcttggccagccagcggaggtccacgcagccgtcgccggtctcgctcacggcgtgtcccaccttaaagaaggccatagaggactcaaacagggtagtcagcgtctccgtgtccagctcgggctcccgacagccacccttcatctccaacagaaccagcttctggagaacgtagcgtgcgtagctggctgccgtcatggtgacggctctggaaaacatctccttcacgttgggcacaaagtagttgtgaaagttggcatagtggacaaacgtggtgactatgaccagagagtagtctccgctctggacgctggtgagagacgggtaactgaacggtcccctcagctccggcaggtccttcgtcttgccaaagagcaggctcagcacgtgctcctctcccttctccgtcacccgcttgtaggtgcccaggagaaaattgcgcgtcatggccccagtgtactgaaacttgttgccgttgatggacagggccacgtaagccagatgacagcgcagctggtagaagacgtggctgtgcggtggcgcgtttggcagcatggccccaatgtagtagaagagctgattcgcaaggggggcgctgagcaggcaggtgggtgtattcagtccactcacgatgccgggcttcaccttggaagcgtccaccttctttgcctcatccggaacggccgggacatcaacaggctcttccagggccaggccaatggtgctgggtgtcttgtagattaatttgtagcggtttatcatagctgccaaatcttcagtaacattagcctccgcccagagtgcctccgggctcaagcccggcacctttgccatcaggtcatgccatgggatggtatactgggaagtgtgcccctctatgtccagatgcaacttgacctcgctgaggctagccaaccccctctcacacagcaacagcaggcacaaaacgcagagcagcttcatcctagtcacgactgcccctctcaaagtcagagatgaatttgctaaagcccgcccaatcaataaactgagatcgaatagccgggttcttcaatgcctgcatataaatctccgtccagcacccagggagatcgtcctgaaactccgagagatccacaagcacaaactgcaagtaggctagcgtccgggtgaacgcgagacagacttccaacaaaaccgcgtcggctcggaaaggctgtatgacttccttaagtacactaaagatgctgttcttatacagcttctcggccacgccacttcgaattatgggggtgtggctttgatgacataccgtggtgatagtggtcagaccggcacacaccttcacaatgtcctccggagcaaagtactgggttagtagccaggcacagtaaacagcgtggtatgcatcattcacactcttcaggtagccggcgtccggccccgattcatgtttcctccctcgcttcttcaggcgacgcaagttctcctccccattcagcttcatccagactatggtgtcgccggggtcggcggtaaacgtggccagtatctgaatgaagtcgctgtaggagaggagctgcccccggagcagcatcagaggaaagacaaccgaggctgacaacaggtggcggtcgtgcaggatccagcaatccaggggagccactggcctggcgccggactctgtactcagcaaactcctcttcctagaggccagcactctgaagggcgtggtaaatttcatctggcatgccaaaacttcggcggacgtgtcttccctcccctgcctggccctggtcacgttcttgtgcatggccttgatggcattttcatacacgtgagtccagtagcgcattggctctggcactacgatggtcaggtccccaaagacagccttcaggtgattgagcatggtagtttttccaacccccggagccccctccagaaacagggaacaggcgggcttgataaccggaacatgatttgtcaggtgggtgacaatgggaacccgcaggcactctttcctctgggtcttggtctggcgggcctcaggggcatcatctagattgagtatattcatcacactcccactcagccgcttcagctgggcggcgtgctttgataatttactaaactcgcgtcgatgggaggccaggtgctcaaagagaccagcgggcttgcccttgctgccattcttctgtttcaacgcaggggaagaggaaggcacgcggaaattggcgtcgtcctcagactcgcagtccgaccggtcatcaagcccaaggcccgcgaggccctcctcgaaggccttctggtaaacaaagctccggctcgtggagtctgcgcctccgtcggcgcacgaggtcttgcggagccaggagaaggggtctggcgtcttgctgggtccgcactcgcggctgcgaggcttgggagtgggggcggtgggcttctgctgctgggccgggagggactgggctcccttgggtagggtaaacgggcacgaggggcgccggccccctacagaggtcccagtattagacgctgcgcccccctgagcccggcacggcaccctctcccccgagagcccgattggagccatcaaggggggccagtgggcaggcacctgatagagtccctcatcgtcctcctctccgggcccggagtcactgccgggcgaggtgagttcggcgggggcaaagtcgtcaatctcggcgtaaaagttctcctgcctctgttcctcaggcgactcgtcctcctggcatttccgccagccacccggagccccggccccttttccaggatatcctgccatggatcccaccaggggcctctgcgccctctccgcgaacgagatggcgagatttcacagcctcccggcggcgagaaaggcggcaggtaagcgcgcgcaccttcggtgttactccaagctcctgtctctccaaaagtgggagcaggtagcctcctttttgtctctgcccccggggcccgcggtcacggactatcgactgtttttcgaagtcacgctgggtcggagaatcgcagattgcgttgtggtagctctgcagccctttacccgctgttatattgtagaatttaagacggccatgagcaacgctgccaacgcgcaaagcgttactcgcagggcgcagaggctggagggcaccgcccagttgtgcgactgtgccaattttctgcgcacgtcctgccccccctcctcgccgggcagccggggcctggaggtttccgcggcgttggtatttaaaaaccagcgttccctgaggacgctccaggtcgagttcccggctttgagccaaaagaccctccccacatccacctccggcctactcagcctcctctcccgctggcaggatggcgctctccgggcgcgtctggatagaccccgcccgactgccgggcgacgcggtcccggagctcatgtgggctcccagcctgcgcaacacgctgcgcgtgtcaccagaggccctcgagctggcggcgcgggaagccgaaagggccagggcggagcggtgggacaggcacgcccagatgctaaaaaataggctgctccgcgtggagctggacggcgtcatgcgcgaccacctggcccgggcggaggaaatccgccaggacctggacgccgtagtggccttctcggagggcctggagcgaatgcgagccgggcccctctccccaggcggccacgccccgccgcccactccctcttcccccgacccgacccggcctcccgccccgctccccgggaacgctcagtacgccgtctccgtctcacccacggacaccccactgatggtggccggcagcctggcccggaccctactgggaaatctgtacggcaacgtccaccagtgggtcccgtccttcggaccctggtacagaaccatgtcggccaacgccatgcagcggcgcgtgtttcccaagcagctgaggggcaacctgaacttcaccaactccgtctcgctaaagctgatgaccgaggtggtgtcggtgctcgagggcactacccaggacttcttttcggacgctaggcacctgccggacctccaggccgccctgtgcctctcggtgggctatctgctgctccaggggggctcctctcaccagcagcgccccatccccgcctcccgcgaggagctgatggggctgggcccggagagcctggagaagattatcgccgacctcagggccaaggcacctggcggaaattttatgattttaacgagcgggaacaagggagcgcgccagtcgctggcacccctcaaccgccaggcggcgtatccgccaggcacgttcgcggaaaacaagatctacaacctgtttgcgggcgccgggctgctgcccacaaactccgcgctcagcgtgcccggggcggagggcagggaccgggacctagtgtaccggatcgccaaccgggtctttggggaggacgtgccccccttcgcctcccaccagtggaacctgcgcgtgggcctggcggccctcgaggccctgatcctggtctacacgctctgcgagaccgccaacctggccgaggcggccacgcgccgcctgcacctgtcggccctgctgccgcaggcgatgcagcgacgccgggaggcggcccaggggggcgccgcggcgccgggccagacgctctttcggcgcggggagctcttccgctacctctgggcccactacgtgaggcccacggcggcggccgagccccaggcttcggtcagctctctctttcccgggctggtcctgctggccctggagatgaagccgacggacggacaggccccctcccagtacgccatcaacctgaccggacaaaagttcgacaccctcttcgagatcatcaaccagaagcttttattccacgacccggccgccatgctggcggcgcgcacccagctgcgtctcgccttcgaggacggcgtcggggtggccctggggcgcccctcgcccaccctggcggcgcgggagatcctggagcggcagttcgcggcctcggacgactacgaccggctgtactttctggcgctgggctacctggcctccccggtgtcgcccagctgagccagctcctcgcgccggagggggtcgttggcaaaaaggtaaataaagtcatcactccgggtttccgtcgtctcgtctctcgtctcgggcaggggggtgagtccgtgccaggccgacacgctcagggacacggcgtgccagaggccctcgtagtcgtgcgccgtccgcgaaggacggcgcggtccaggagcaggtaagcccggccaggcacaggaagggccacgaagagggggcggaggcgggcccgaacgcgggcttcgtgctcgtccgccccccagtggacgaggcagtagagcacactcacgaccaggcggttgggaagaacgcccccgagattgaagagcaggttgccgtccgccaggcagacgcgactcaggtccggggcacggcgcagcccgagctgggcccagacgccggcgcagacagcccccgcgacatccggcctgtcgtgcagaaaccggcagagggcctgagagcgggcgcccgggcgcagggcctccggcgggaagcccacccgtctcagggcccggtagagcaggtaccagtactcatccgggaccttgcgcgccagcacccgattcgtgcggtttccagtatttatcacggcctcgcgccacaggtaaaagttaacccccagggtcagcagcttggtcagggacaggtgcaggaacctgagctcgtcctcgcgcagcgcgcggagcggccggttctcgagcagcctcaggaggagcccgcggatcccgggcgtcagccgggcgtcgtccccgcgcaccccaaggcccagcaacatcaccgccgctgccaccgccgcaatggtgcaagctagcgaggagagccgcccacccagcgcggccgtctacgtctgcgggttcgtggagcggccggatgccccaccagaggacacgtgtctccaactggaccccctggtggtgaagggacagctccctctgaaaaagcccctgcccctcaccgtggagcacctacctgacgccccggtcggctccgtctggggtctgtaccagagcagggccggtctcttcagcgccgcctcgataacctccaaagactttctgtccctgctggactccatctaccgagattgcgatattgcacagagtcagcgcacgcccctcccccgggaccctaaagtggaggccctgcacgcctggctcccctcgctgtccctggcctccctccaccccgaggcccccagggccgccgcggacgggggggagatgcccttcttcgaccacgtgtctctctgcgccctggggcgccggcgcggcaccacggcggtctacggcacggacctggggtgggtcctgaggcactttagcgacctggaaccatccatcgccgcccggatcgcagacgaggccccggctccgtctccggaccgcgagccgggcctccaagagcgccccctgccccttaccaagctcatggccaaggccattgacgccggctttctcagaaaccgcgtggagaccctcaggcgggacaggggagtggctaatatcccggccgagtcgtatttaaaggccagcaacgtccccgacatccaaaggacgaaggacagcgcgcctcagagcccaccaccagtctccaggaaccgggccaccatgctatcgggtaacgcgggagaagcagcggcggcatgcggaggttcggccgcccagggccaggacctcatcagcgtcccccgcaccaccttcatgaccctgctgcagaccaacctggacagcaagcctccgaggcagagtccgctgcctttcgcggcccctctgccccccttcacccaccaggccattgccgcacccgcctacggggccgctgccgccccgggtggctactttgcccccgcgggaggctactacgcgccgggcggcgaccagggggccttctccgccgcagatccccacgcctaccaccacggctaccctcctccggcctactttggcctgcccggcctctttggcccgcctccgcccgcgcctccctactacggatcccacccgaggtcggactacatcccgggcccctcgcgtgccaacaagagaaagagagagcccgaggaggacgaggagggcggcgggctcttccccggagaagacgccgccctgtaccgcaaggacatcgcgggcctctccaaaagcgtgagcgagctgcagcacgcgctgcaggccctccgccgggagacgctgtcctacggacacgccggagtcggctacggcccccagcaggccccctgctacgcccacaccggccactacgggccgcagcagcctcagcctctccttccccagccgtgtcatcagggtcaggaggcgcccagatacttcccccaccctcctccgtcgtttcccgccccgcaggccgctcagcctcagcctcctcccccgggcacacagcccccgccggcccactgtgcggccgaggccgccgggccgtcaacggccgccgtcccagaagccgggagctccgggcccaagcctgactccaagccccagcctccagccgaaacccaccagcgcgagaagaagctggtgcaggcctctgcctccggagcggcgctgcaggcccaggagcccgccgcctccaaggccaaggcagtctcagcccacctcaagtccatcttctgcgaggaattactgaataaacgcgtggcatgaacgtaaagtttattgagtgtcagtggtgtccattcacacgggtatccagcccttgcgccgcctcccccagcccatcagccacccacagcaggcaataatgatgactaggattaccggagccaccacggcacaagttatgaggagcagggcccagtgcacccaggtgggtctggggtgccagggatcgaccgggaaagggcccagggtaaccgggcgccgagtgggacgtttggtggtgggacgccgggtggtttggtgactggtgcttgtggcggggactactgtggtagtgggggctgtggtagtgggggtgatggtagtggccgggataactgtgagatgcattctcgcggttagggtccggttagcatggtattgttcatcgtcgtcctccgccgtgtaattgcagtggaccccggaggccaaactgcaattcaccagggtcacattacatatgtagtccccctcgtgttcacttgttaggttgaaaaaggtcagggtcactgaagtgttggtggaatctgtccattctagggtgagagtgtaattgtgatgcccactataaatatgagttccattgttggaagtggctacgcctagtgttggaattccatcaccgggagtggatcccttgcccacctctatatgagaaacagtctcattcttgggaacactgcaggtgatgttgacttctgcacctgcaggggcggtgacattctcaaacttgaccaagtcagagaaaaaggcacaaacagaccccacacagcagcacaatagaagagctaaatgagtcattcttaactgacagttttggaagccccatcatctcaggcctaaatacgtggtggggtgagattaagagcactttcatattctataatcgcaggcttaaccggaagagaagctgccggttgccatttttcaagcagcgcagcagagaaaggcagcaggggaagaaggctcctcaaacagcatggatctcctcgggttaaagaggcacgggcaaggagtcacgtagacacgggccttttctgtgaggtaagaaatcactaaaaagagcccaggatgtgtaaacagacttttgcgggggatggtgggccagacacggtgggtgtggaaaaaggggctgaaacacgcgagtcgaaagggtcccaaaatacaccaggcatcaggaattgctcaagccaaagttgctcctctgatggctataggcccaaagtatacatttgcgtgcttggaactttgctaatgtgcactgtgtttcctgtccgcatgcaaatactctactccctcagcctagtctaagatgtctgccacagagccctaggtgcttagccagactgtcattctattggtaaaacgggcagaaaacagaaaagacccgcgaagtttgggcccttcacgatgcccggtggtccccagtagccccggttctccggggccggatgccgcggtcccgcacggtcgctgcaggctcgcagctgctcagcagctgcaggcgggggatgggtagcagcatcttagtacaggcctctcatcacaggagaggtgggggtgggggtaatgtctgaaaagtgagcaacgtggcaggtgccgggactgaaaacacgcaaaaaaacacccgcggagggggggggtggtggattatctctgggtcctcggggggcggtggagaccgcgggtgcggtgattgcttgaccctcgcacctcgccgtctctactgctcgccgggtacgtctggcgtctccagggtggttccgggtgggggggtggtcccgctccgagagctttaagccgggtagaaacaggagaaatggagatgctttaaccttggggggacctagtaattgtgcggtgttgtgagtgttagaaacaccacacgatccactaggtctcacctgggctccttgcctcgttgcccccagcccccctaccctcaacaccaatacaccaaataagtggggcctctttcgtgggacttgctgagtatgatgcactgtgaataagtgtcaagatcacccctagccaacaagtctctccgggtctgcttgcctcctgctcccacacaccagaccaaagacagggggtctctataaccttcggtggaacctagtgccggtgatgtgctgtggataggcagctagcgcaccacttttcactaggtgtcaccggggctacttgcaccgcggcccctccgtctttataacagtctggaagtgaggccctttaacccttggtgggttttagtggaagtgacgtgctgtgattaggtacccatagcaccgctatccactatgtctcaccagggctactcgccatcgtatcccccattgcggatacggcccaactgcacctggtgtaattcaagcaagactctgtcagtatcgtcgtgccttccgctaatcaccgtgtgccgcttcgaggtaaacatcggcttactgaagtctcttagtaaggtcagtgggcctgtttccctcacagagacacatggcatgccaactgctcaggggagggggtgaggatggtgggcccagacgcagcctcttcattctgattgctgcggcaccgtaaggtgaatatagctgcccattgatgtctgtttagaacccggtgggccgcagttcacctacagtgacgcagtgactgccagccacaacgaagagcatactcaacgttcaggtgtggaattatagatagcgtgggtgtgtgaccttgtttgttacacaaagatcaccaccccctgtctgctttccactcctgattagacttcggaggtccagccgtggaaaatgtgagctttctgcccctcctaacacctagggccacccccttacgcggcctgcggaaccctaagaggggggcctgcacgccgaggttatttcccagcccttcgatgcatggtccccccttagtggactgcaggcccatgtagcctcagagaacccccgcgccttcacacaggtccccctacgttgaatgacaatcatgggcccatgttgcattgaccttgttggttaccttaaagggtagtgtggttcacagcagtttgtgaaaaacctgtggatctaacttcccttagtgtaacccagggtggagcagcacacgtgccactgctactcaaacaaggctgtagcctttagatggcgtaacaggcaacttgtgtatgaggccaagttagcccgaaccgctatccatatgctgctgctccatctcatcagatgtccaggatcgtcccaggtgtggaacccatcctcagcaggctgagctgtgcagttttgattgtggggggagggctctcttatctaggctagaggcctcttctcacaattctaatttcacagctctccagaagccgccctgcctcctccccggccggcttgcgctcccggggagcccaggtgtctgagaacttgggccatacatttacagggcccagattactgttagccagtttaagtagtgcccaggccacaacccccacccagcagcgggtcccccttctagtctccttgcggttgtgcttgtcctagtgacagcctggcattctatcagaataacgtgaggaaaccaggggaggactcaaccactcaaccctccccaggtatcccgctgacattgggccctgcgcaacggcaggtcagcccggaatccgaaacatcacacacccagcccgtaggaaacggcccaaccgtcccgtggtccgcgtcaccccaccacccccactccacaagcaccacagacacgagatgtaagtttgtacactttattgttaaacacacgggcccctcgctttcccagggtttgggtttgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcggggccgggggccgggtgcccctgggtccgctgccccgctccggcggggggtggccgggccgctgccgggtccgctgcccggtcctggagctcgggccgggggccgggtgcccctgggtccgctggttcggttcacctggaaggcaagcgggttcgtggagcccccacccccggaaccccagccccatcccggctccccctccggcctccccccgggggtgcccgggcctacccggttctggagctcctgggggtccgctcgtccgctggtccggtccgggtgggcctggtccgccggatccgcttcgggggtccgccggtccggtgcacctggaaggcaggggggctcggggtgggtgggcgtgtcccgggccccgccccgggccggcagggggtccccgtggacagggccgggggttccgggggcagccgcgacccagcgcgccccgttcacgggggaggatcgcggccgggcctcccgggggcccggcggggtggggggtgcgcccccagccggaccctggtgccaggcagggacctcgcgccgcccgcttcatggggggggaggccgccgcaaggacgccgggccggctgggggttgtgcaccccccgagcgtctggacgacgctggcgaccccggccagcgcgccttcttttattcactttttgggggcgctgtgcaataccttaaggtttgctcaggagagggggcttcctattggttaattaaggtgtgtcattttagcccgttgggttacctgcaggtgtgtccacaggtgggtggtacctggaggtgatgctattgggattaacgtgtgggggaggagctaaggtccgcgagatttggggtagcggagcctcaggagggtcccctccatagggttgagccaggagggggagaaattctgcgccccaatatacctagtgggtggagcctagaggtaggtttccatagggttccattatcctggaggtatcctaacgccgcccctatattccaggtgggtggagctaggtaggatccagctaggtttctaccgggtgcccccctatcctacctcgaggtgcgccacccttcctcccttcgttataatggtagcataacctataggttattaacctagtggaggaatagggtactacagctgggtatatacctatgggtatatacctataggtatataggaacctagagaaagggaaccctatagtgtaatccctcccccccctacccccccctccttacggttgcctgagcccatcccccaccccagcaccccggggtgacgtggcacccctgcgtgcttttcaggtgcatcacctttgcacatttggtcagctgaccgatgctcgccacgcccgggtcatgacctgggccagcgcctcgtcccgaggcccctctgtcctcctagcgtgtcgcctgcctccgggatgcatcactctgagccactaagcccccattactcgccccgcttgcctcacggtggctcactaatcccccgctaatccatgtgctccgcccttaggaaacaccgcatcccaggggcggaagcgtggacccagagggcggaagctggactttggccacgggcctcgcctggagttgggttgcgtggggtgtcaattgggggctgtgggccgaacgggggctcccgtcccccgggggggcgcctgtgaggcggctgttgttgtcaaaggggcggtggcagccgcggtcccccacccttccctggtccctgggctgtttgcctgaggtgtgtttgtgactgctgaggtggcagagggcccggccttgagcccatcccccgtggtgtcagcctgtagtggccaacacccttggttttgttttgttcctgttgctggcaactcactggcagcagagcagcctctaatggtcgcctgcttttctctctctcgttttatttatagaatcacagccaggactggtacctgagagctgagagtctggtacgtggtttgagaccccaagatttgacatgcacacctgttggtattgcttctattcttgaacgtgagagtacttagtaaaaatgtctgcaaatatcactaatttctcttaactggttagggggtatttaagaaaaggcaaaagaagcctctgtattgcttggccaagtcacttctgtggggctaacacctcctgttcctgttgtaaaaggggccctcagtgtgccaggcttgtggttaaggtcccttctgtgccagctgagagcatttttctgtgtggtgggggtagctgcagttcagacaagtaatgactgaggggctgccattgtccattgtgaactttttaatgacagcagcatattcttcaaacagctaaattttaatttgcctaagatagcaatgtgagatgtggttaattgaccattttcctgtctggcataaatgcatgggcgatcctgggtccatggcacatatggagataaacatagatgccgtctggctcatgggcaggtgtgggcaggtgggggtgttagtgtgcaaccctgtttctggcttccaaattgggtagggggtgctattttctctatggaattaaaatgctgacattagacagtggagaccacactatatgtggggatgtttgtggcttgtcatttcctctttgaaggtaatcccccaacttaacttccctttaaattatgatgcaagccttgggttattttaggatagttatctaggcttgatattcgtggggggaaacggtggattttggtgtagttattattcaccgtttcatccccacgattattcggtttcaattgctcttctggtttcacgggataactttccccgaacacacctttcccttcattgtctgcagtatcccccgtcataacttcctcttgatttgttcagagagatctccaggatgtcagctctgtgttggggcccgttggcaggaagaggttccagtcttgtgtcctattttgggactggctgtgggggctgggtattcactagtgctggcacctaagataactagaattgttagttgtctgcactggagagtattcggtttcagaagtcatcacataacacgggagggcttctcctctcatcctttgccccctgtttgttggcagcaccagcgaagcccaggaggggaccacggcgctccttggtgcctgtggtgatgctctctttaatttatgcccttctctaccatagtgggggctggttctggctcctgctcctcaatgtgcccaaggtgcaggggttgccgctctcggatgcgtggccatgcccacccccctgcatgggggcgctctgtgtgccaaatttcgcgagctaccccttgttttccctcgttatctgatagcatgacagctaggtcacccaaagtggaagccgagggaggtggctcttcctttttaacatctagagaactgcccatgttcctgctggagttcctgagtgtttactggcagtggtcctctgacatcgggtttccgctgtacttgcataatgagaactttgtagtgcgccggtgacctgatagcagcggtctcacaaaggcatcgtcgtcttgtcaaacgtgcgggtgccctggctcaagttctcatttccaatacagtttgagaaccttgtatcggagataggacttgataccagtttgcccgatgcttccagcagctgcggtggttggttccctctttatgtagacaccggggccttgcctccgtatcctatcaagtgtgacgtggctttgttcgtgacactcgataggatacgggggtaaaggcgcctgaatttcagtttagctgggagcattggcgggttcagcctggctgagcccccggggtcctgtcccagctttgatgtgtttttaggtttcacggggcagggctgggtcaaggcacttagccctcagtttcagggtacgctcagaccagaccatgcacagtgggcgattaagacattgctctaacggtgcatggactggctagagtgttcctggagtatgctggaggtctgccacttgccttgagcctaattctgtgtttttgcaaatgggagcagcgcctcacccctcagccccctctgttccttggtgcatgcatttcttatcggtggtgggagcagcctccggaatgccattttcgggggtccttttaggcagcggttttcaggtgacctatctggagcagatcccgtgtcttgatcctgggcatgggctatgaaacagttctaacaattggatcatagccagtgtccagggacggtgcaccgggaaggctcagcgtctccaggctgtacacggttgattgaatttttgcccctctgtgccctctgcacacctctgcttttgtgggcctctagtctcacagagtcgggggacaaccgggacgtgtcttccttggtccgtgcctaggagcgccgctgttccaggacccggcatgggtttctctgttaatttgtcttcagcgctgccatctatacccctcgagtgccaattttagcgtgccttcctttctcctctcgttaacctgatagcatgaagggcaggttacacaggctgcttactgtggattgtaatctgcttatgacagcaccctctccactgaaacacggaaagctgggctgccccaagtgagccctcggccccagggtatgctcccagtggctgttgcgtggccctttggggatgatgtggctgctgctggtgctagaccctaaattggaaacaataatagtgattctgtgacaatttaatgggtctagtagttgcagtctggcccctaacactcggaaataaatgggctgatgttttcagtatgttctccacagggcaggtgtgaagcggctgtcgctggcgctacacccgggattcgaagcagtcattgggcctctgctacgattctccgggtttagcagttgcgctcccgacttgcaactttgagatgagggggtgctgctttctggtttctaagcaggcggtgtggtggggctcccgtggagtgacccggagccgcctccttggttctgtccacattttgttgctgcacataaccatggaggtggttgcggtgcagcctaggctaggcggagcctgcgtgcggattggcctgttgattctcaggttgaggtccgcatgcccaatcctggcctcaagatgccccgaagcccatggcgtgctcggctatcgcagccacctgccctccggctgggtcggacggtctggtgcgcttgatgtgttttcggtaccatcgcacatcaggctgaacgactcagaggagtggcccttgaatgcatgcgggggttgattctccggttcatgttagcattccccacccgggcctcaagaatgccccgaagcccatggcgtgcttggctattgcagccacctgccctccggctgggtcggacggtctggtgcgcttgatgtgtttttagtaccatcgcacatcaggctgaacgactcagaggagtggcccttgaatgcatgccagggggcttccgccctagcccttggccgtgggagcagctcccggtcccctccgcttgcgtgccaattttcgcaggccgttctcctttctcgctcgtttatccaatagcatgaaaggcgggtagccgagcattgttagtttcctggagccggtgacctgagacccacccttctcgacggggcaggtgttataagtgtcctgtagagtatgggtgtggttttagtgcacgcgggagtacgtggaaatgatcttgaacccggtgagctctgggtgccaagaaaccaggctggcccggtgacgctggcccggaacttccctggccacccagtggacccggagagtctgtcttggaaggcgttgctttcagctgtttatatggttggaggaaagtatcgccttctagaattggcttgtccatttcttcgtgagcctgctgagtccgtgtcctaaaaccagtcccagcagacaaaacatagtatggtcgagtgttttgtttgcttgggactgcagttagggcgcgtaaactcgtctctcggggtgatgccacgcgtctgtgccccggttgctgctgtccagacgccccccggccttgccgcgccacccgctgtttttggagggccagttgaagcaggcatgtcttcattcctgatttcctgatggcatgaaacacatggcctgttcctattggcaccttcactcacgccgcgtggagcatcaggaagcccgtgtcctggcaccggtcctttgaggtacgaaacattgtgagtttgtttcgttttcgaaggtcggctgtcagtgcatgtggagattttcgacgctagaatgctaaccagggctccccggtcaccggctcacagctagtacggacgattattggctctgtaacttgtcgtgggacgggcatcgatgtgctcggcctaccacgttcgacactctcgccgttcccttcgttgcccctgccgttaattcactgccgggtaatgcccggtcgcttacgccgaccgcgtaacttgtggtatattgttaggcgtgtcgtcgtaagcagccgggcgacccggcgctcttcttcactgggatctgcgagggggctcacagtcggctcgggcagtcattctccccacgttactgccccaacgcctaagctagaactggtactggcgccgcaacctgggacgtcgtctgcatattcacccactcttaacccccaacctgcccggctaagttcagatgttctctccatgttccccttgttaaggaatagcatggctgtgaggtttgttctccagggctgtttattgagatgcggggagacctcagaggcactctcggatgtggggtgggggatctatccgtgcccggtcagcgttagcagacgcacttgccccactacgcccaccaggctaacgctgtggctgtcttgcctgggtgtgtggggtcccaggcaagcccacacatctcaccagacacgccaagtggtgtcctgtaacttactactgccgctttacataacgtagcttgtaaatgcgagcagtagtaggcgtctggacgctagacttgttcctcttcaaatgatttgagttattgtggaggttagctagttttggtctccactgcccccttctccaccttgatttccacgcctccagatgccggcccctccacccaggctaattccgccacccgtgcccccacctctggccaattttggcctcggtgcttctccacgacctggtcttggttcccaggccgccccctccgacctaatcccctccaggccgcttaagcccccgtgctctttccgaccgggccccggtgcctttccatggaccaattgtggcctccgtgcctcccctcccggctgtttccggcccacgttgcggcgggccaattgcagcggctctcccccttttccccttgttaatagatagaatgacgtggaggttacaacccagtggcagctttattgtgagaccatttacagagtgcccttggaggcgacgatgttcccgttaaaagtcttgttgattctctcgaaggtgggcagggaggagctctccaccacaatgttcccgggcagcgtgagcgcgcagccctggcgctggatgtatctcctcatgagggtgccgagggatgggtccccggcgtagatgaagaaggcctgggccaggctctggccggtcactatctctatgggtttgttggagatgttgcggacggttagcttgaggggctggcccggcttccactcctgagggtagacgtagaagaccgggttggtggggtgggacacgaccagcgccgtgatcttggagctgaggggggcctcgtaggtgttgttgtattctagctccgtgatgaaattgggaggaatgatcacgggggagccaaagtagcgaatgtctgtggactctccatcccagcgccaggtgctcttggggtaggggttgtagcggaaggcaatgatcacgtcgtccaggagtgtcatgcccaccttgacgtttagagggccctctcgtttcaggtccggcgtgtccacggagactctgacgtagcccttgccgcggcggacggcgttgaccggacacacctttcccgggaacgtgtggatgcgcgcgtaggacttcaggaacggagtcgtgtgctgcgccagcaggtaaggcaggcactcgtcctggctggagacgggcgagccacttaggaagatctggggctcgctggtgttcaaattgtccccgctctgggagcaggagcgcgtcagcttgatgtcgctctggccgggcaggatctggaggtagaggtaggggttcttgaccagtctgatgggcactatgtaccaggtgagccgccccttctctatgaagaggcttcgtgggttcagcacgctcaggacgatgtcctggtccgagtgcatgacaaagaagggcttcaggaagacctcgttgtcctccgccccaaagaacagaaacgagcacgtaaagtagcggctgccgtaggtggtcgcgttgaaggagaaggaggggaacacaaagctgattatctggcccaccgaggggcaggggggcagctcctggcactgcgcgtccagctgcagcacctgcttgttggtgacccggacgtacgaggcaaagatctcgtatttccacacccccctcacgaagctcgtctctggtttttcagcgggacgcaggcggcggaggctgcttctgaatgacgagcgcctggacgctagtgctgcatgggctcctccggggtaagcggcggccatggctgcagttcttccacgggtaaggttagagtcggtggacgcgcgacgtggcggccccagtggagctctccgctcgttagctggtagaatatccgtccggcgtctagactgtcgctcctcttggccggccagaagagcgcgaaaccgagcctgactcccgggcctacgtgcagtggcttgtccccgcagttgtagatggccaattttatgggcgagtgggcggtccacacacgcgggcgcagcacccattggttgcaggcggcctcctggaacgtaaacccggagagcgaaggcgccgcgccctggagctggccctgcgccactacttgtagctcctccctgggagggacgacaacgtcgcgctctgggtggaggggaaacagcgtccagcccccggccagggtcacgaggcctgggtccatggtcagaaaacaaccctcccgggcggtgggcggccccggccccaacagaacctcgcaggcccagccctcctctgccctgtccacgtgcacgtctaggcggacgacgccagaggccgagtcacgcgacaggtacaggcccggtgccagccgccctcgacccgcgtcccaggcgaggccgcgccagcgtagagcccgggcctggctcggggccgcgtcaccccgccagggggacgacgcctcgccggtctcgagttcctcgcgcgtcctctttcgccccctctccggctcccccacccgacccggggggaaagtcgcccgcccttggggcaggaggggctgggcgcaatcttcgccccccatcgcttccctcctcgttagtttgatagaatgacggtaccacgcaatgggccaggtcccggcgtagggaaaggccagggagagagaagtgggcgagctggggctcagcgggccccagacgcgcaggccgccattctcggccgcggcagttgacccggcggcggcgcagaccggcgccccttcgcccggtgcgcagaggagggggagatctaggacagcggcacccacgcaaattagacacagacacagatcgttggggttgaggcgacgcaggctagtctttatggttacaaacatgggagcgtgcacctggaagacgcagctggggtagatcttgacatctttgcgcgtgtcggcggccgccagacacgggagtcccgagttcacggcgggcacctcccagagcgagcccaccagcccgtacctggccacggccagggcctcgtaggccgagacgggcagccggagcttgtggtactgcccctccggcaggtccagcggaaggcagttagagaacgccagtcccctagaccctatttccacgcgccaggcgctgaggtcggtctgcagggccatactcgcgctcaggtggactgggtggacgttcctctgaagaatttggcggtgactgataccttgttcttgagggagtccataaagtggttgaagctccaggcgtagagaaggatgagcaacagggccaggaaggcggcggggcctgggtaatagcgggccgcgagcgactctgtgcaagcgaagcccaggctcccggcctctcccaggaagagcagcggcagtgacagcatgaaccaggagaaggcgcagatgaggccggtgaaggcgacgttgcagatcaggcgccactttcttccgaattttgtgtgcaggagtttccagatgatgatgagggtgaggatgacaatgaggatacccgccaggagatagcagccggccttcattccctggatggtcgtgtgcatggccctcgtggggccttccctgcacatgttggggcctctgttgagatcggcgttggggcccatgatgatgaggaggatgatgatgagcaggagcaccagggcaaagaccccgcccaggtagaggtacactgtcctgttggagtgccgcatgggcttcgtgctggtcagggcccgggtcttctctacggccatgcccacgtgcgtccagacggagcacatcaggcccaggaacatcaggttctgggtcagtatgcagaggccggtggttgagaattcctggatcattttccccagcacccagaacaggagctccagcaggagcgcggagagccagacgtccattctatgcgcaaacttcctaaccagggcgatgaacaggaggccgaccacagttaggagaatgagcagaatcaacagcaggctagtcatgccactgaggaaggcgctgtatgatctggtgcaggcatcgtccgtggcattgacgaaagtcatgttggccacctgggtcaccacggtggaccccggggccagggtggagagcatgctgctggccactgccatgatgggtggtggccggtttgatgtgtgcttgaaaagaggaacttgcctctgcagcccaagaggcactttgcatgccaccctccctgtctgtggacttccagaccattgtcttcatttgcccttgcgtgtacatgactgcaaagagcaaatggggggaatagcctgtgagttccgcttaatcgccggtttccatagcgaccttgccagagtagggatgagccggggcgcaacagcacgatccccggtgcctttcccgatgatgacacccgccgcggctgtctgaggcagctgaggaaattggttgagattctgacgtgggaaaggcttcggtggtcttgccacgcaacaatcccttggggtcttgccctccaccagcttccccgccacttctcggctggaatctttgcctccctaccccgctaggggttttggcctctgccagcttccccgcccctcctcctcggcgggaatcttcgcatccttccccttgtgggggtggctcggctgctctccttcccgctcttctctcgttaaaacgagagaataacaacgggggcgcgcgcccggcatcccgctgggcttagaatggtgggcgggcggtgaaattctggaggacggagagggtggccgccgagttgttgtcaaagaggcactgcaggatgttggccgccccctgcagcagtttgtcaaagtagtggtcaatggccacgggaatgccgtgccgctccgcgtaggccgggtcctcggccatctccgtttttcttgcccccttgatcccccccttaggctccacgaagacgtactggatacggtcgtgaatttggggcagctcctcgttgcgctcgacgaacttctggtagacggccaggtgaggcatctgggtgctcttgtaggccgagagcttgcggctcagctccgtggagaagcagagctcccccatggggaccctgccctctctgaggtccgcataggcctcgttgaggatgtcgatgaccggcaggaagcccgccggcagcccctccgagaatgactcttggaaggggcggcgggagaggaggctggccgcctcctttacccgggcgtccgccagcaccaggtcgagcacgcgccggcagcgcgcctgcacgaacttgcaggccgttttcctgaccagctccacccccttcatcaaggtcttgccgtccgtcagcacccccacgtatctctttttggtgatgagcatcaggcaggagaaggtcttctcggcctccagggagatgggggccacaaacaggctccgggtggtgtgggcggccagggcctcggcgaagcgcagcgtctcgccctccgagaatccccggcactcgataaacagcgagtccgtgtccccgtagatgactcgtagcctgccctcggggttgaggggcgcccaggcgttgggagtgggggccagggcctgcaggtcggccggactcagggcctccacgaaggccttggcccgctccagcatcgtgcgcccctgcagcgtcacggtctcggcgatggagaggcaggggaagaggccgttggccaccccagtgaagccgtagacggcgttgcacgtgcacttgatggccagctgctgcttgtccaggatggtcttttggcgcgggtcctcgcaggccgccagcagcttcttaatggccttgcgcttggccagccaggaggtcagcagactggccaggaaggactcgtgcacgtgcttctttacaaagtggtaggtgcccccggtgagcctgaaggactcgtagtcctcgcccgggcgcaggccggctagtctgtgctcctcttccggcgttatcatggtagaataacagagattatgagcctgaatgatgctcgggtagaggctggcaaagtccaccaccagcaccggggagttgtagaagccggacaggggctggatgacagtggccccctggtagccgtccctgtcggaggcggagggcatgggtaggataaagttctccttctgggccgcggccaggaggcaggagaacacgcggatctgctggccatcgtccagcacccgcctgcaggggatgtgggcgatcttggcgatctcggccacctccacgtggatcacaaaatggttgagcagatccatgaccagggccgaatcctgcacgcagtacatgcccagcctcctgcgcccccgggggccctctgcaaagaggcgagggatctccttgtagtgcacgtcctcctttttggcccccagcaggtgccgggccaccgtgtccagcttgtagtcagagaggctgagcttgtccctgcacacagcgtacatgtcaatggggatgaggccggtgatgcggaccttggtgttggcccggaggaagcccttcccggcgtcgtggggcctcctgacctcgcagacgcccccggccctgatcttgcccagggaggccggattgatgccgtagatgtgccgggccctgtccaggacgtagggccagtcaaagttggccacgttgtagccggtcacgatctccacgctgaggtccctgatgagctggaagaaggcgtagagcatgtccagctccgaggggaactcgtagacctcaaccccctctatgtcttcgcaggtgcccagcgtcagcaggatgcggcggtagcggccggcctcctcccccgtggaccagaggacgcaggagatttgcaggatcaggtcagcctcgttggtggccgtgggaaagccctcctcccccagacactcgatatcgaaggccagggccgtgtaggagggccaggagctgtcctcgcgccggaccgagaggtcgcccacctcgcagtcatactcgagctcggcgtacgagtcccgctgctgcaggcgggggatggcgcggcggcagctgtaccagccaaaggtcacaaagtcgttgtccaggacgaagcggcgcgccgcgtccacgttggcttcgaagatctggcacccgtgcttgtcctgcagccacgtggccacgtgagacacgctgttggggtgggagagggtgattttgtgatagacgccggcctggttgccgtagcccatgatggagcggcgcgtgacccgctccaccgagacccggcagggggtcttgcggtcaaaggtgcttgccctgagggcgttgaggacggcaaactccacgtccaggccctgaggcgcgcctgcgtagaagtaggcctgctgcccaaacacgttcacgcacacgctggccccgtcagccttgcgcctgcccagtagcttgatgacgatgccacacggcactatgtacccctgtttatctgatggaatggcggcacatctctcgtgcgtgtacaccgtctccaggatgtcgtagacgtggaactcgagggggcccccactggggtctggctccggccctgccgccctctctaggggccgctggcgccaccacatgcccttcccgtcctcgttaccccccaccaccgtcagggtgtctcggtagaagcacagtgggggctgaggcccccgcacgtccaccaccccggcagcgcccggggtctggaagcacttgggaatgagacgcaggtactccttgtcgggttttttcaaaaggcctttattaggtcttaggaaagggttatagaagagtcccccagacatagttaaaactcagtctctgcctcctcgagcagcccaacagcggtatctggatcgtgatagcgtcgcctgcgaaggccgggcatgcgatccctggctgcctggagtgctctgctggccaccgaaggtttggtcgcccgctgagctgctctcttttgctcttggttttgctcatgcagcgctaacatgatggcatctaattccggcttactgatgggagtaatgttttgcccctggccctggcccatggcatgttgacgagcgagctcgtcgatccccgggtagagcatctgcaccggctgctgcgacatctggcgcgtgcgccttgtgagggtgatgactaggatcaccaccccggccaccaggaccagggtgagcatgccaccaaaggggtttttaaagaaggagatgaagcccgagaccaggttgctaaacagcccccccacggtgctgactagattggtaataacctgacccacgctccccaggctgtccatgagctcccccaggccgtccacaaattgatttctgttgtttgacacagcattgtccaaatctttccgcagaccggcgatgttctgggcttggaaattgtactctctgaagatgccctccaggtcgaagacgttggaggcccgctgctcgtcgcgcgagtacagctccagggaggcaaagtcgatgttctcgatgagggaggtgtttagggcgatgaaggtctgcagggtggcgatgccgtccagctcgatggtcttgaagtgttggtagtcgtggtagacgtggatttcgttaccagactggaagtagtactggctggacgccaggcacatctccgtcatcttcttggtgaggaagatctcgttatccgtgcccagctggccctcgtaggtcttggtgtcgttgatgaagctgaaggacaccagggggcgcgagtagcacatgctctcggagcccgggaccctcatgctcttgcgcagtgtgaccgtggcctggttgacgggcacgcactgggatacggagattacgtcccccaggcgcttggccgccacgggccgcccgtagatgccggacatgacggtggtgggattgatcttggtcagctccctcagcaccatgttctgccttttctgctccaggcaccaggcccgggccaggtcgcccagcatccgattgatctggcggcgcagggagtcataggcaaattggatctggatggtggccggattgttgagggtgccaagggagttcccgggggcgggtgtggctggctttggaggggccggtgttgtgttgttgccctccgcctgcctccgtcgacggcgcacaatggccgctgccgtgcttctggaggctgtggatggagtggggggcgtggggctgcttgggaacggtggagctgccgtggtcagctccgtcaggttcttgacggaggccaacgagcgcggggtcagaggcagccaagccaataacaatcctccgctcgttagaaaatatgtaatgttttcctggtccttcacatagcggtcctggagggcctcgtacttctcgtgcatggtcttgttaacctgatcttcgatgcacttgaaggctccagggagggccatgcccaccgtcatgttggtgacgaagctggatgtgccctcttgggtcacaaagtgagttgacctcaccgtttctgtggcgattgtcgattcaaaggtcttccagtgttgcagtgggcagtaggccgttctgttttcgagcttccaggagagggtgtaggtacccttgtccaggaaggcccgccgctccccgttgggcacggtgcctcgattgtcgtagtcaacaatcttgtaattttccctgacgtggaaggagccggcccgctcgttgaaggtctccttgtttgtcccgtcgtagaagggagacatctccgcggtctgtccgaccgtggtcacgaagaagttgaaggggctgttggacttggccatcatgtcggtgaccacgcagttgacggtggttctggttctgtaggtccacagtatccaccccggggcgtcgtggacctgcgtctggctggcgtaacgccgcacaccgttagccaggccgctggtgggcctcaggttgacggtgatgttggtcccatcgttgtccacgtacacccgctccaccccgtcctttgtcatcctgacggcgttgtggcactggtagatgctgtccatctggtcggtctcgtggctctccactgagaacatctcctcgtgcctgttggtcacggcgtccgcgtaccagccgttgtagacgagcgtgttggtcactatcttggtgtaggagcggaccttaaaggagtagggaataatgttgtctttgaacaccatcaacaggccctctgtgtgtttctcctgcgtgccggacgagggacattggatgtccgaggagaagcggaagaggtcgccgtggctggagagctcgcagacgcggaaggggaagttggtcttttggcgcgctgccgccggctgcaccgtggtggcggggggcgctggctgctgggtggtctgcgcccagatgcagcccaccagggcggccagcagcaagacaacgctcggcttcctacgcggagtcatcttgaaccatctggaggtgcaggtagagcagggcatatagatccttaaatacccaccccctgcctttatacaggagaattaatgggcggtcggaatcgtacgtcaggtagagcccgtcggggggcagggcctcgcccgggctgaccgcgtccgcccttcgcaggcgcagggaacgcccccaggtcttgttgtagagggagacggtcagcacggcctcgcgcagggcccggcacagaagttgctggctcgaggccagcgagccctccggcacgctgtagaagctgttgaaggaggtctctatccaatcgctcggctcgatgccgggccagatcagggaggtcaggaacaccttctggtggggcaaggtccccgcggcgtcgcagcagcgggccagggccacgttgctgggcggaggaaacagccccctctcctcggccagtggccccgtgatgaaggtgtacaggctgtgggtcagggcgtgcaggtgctcggcgctcagcgtctgagtaaacaggtgggtcttgatgtacttggaattctcaaaggccgcgccctcgccgacgcgcctgtcctcccagggacccgagacgaaggcccgcctgtagaggaagtggttgcgcaggcaggccatctcccagtacaccacgtccccccacacgcgcaggcacagcgtctcggtcagggtctcgctctgctgcgccaggcaggactgcagccgggccagcccctctgaggccacctggcgtaggtactgctccttgcgcttcagcgcgtccgagagggcgttggagccgggctcgcgcggggcgcccgccaggggcacaccggggctggcctccggcccctcctccccctcgctccggttcagccgctgcatggcccggttgagccgcgtgtacagctcgttcctcttctgcaggatggcccggtactgcgggttcgaggtgaaggcggccgcgcagtcggccttcagcgcttccacggcgtcgcccgaggagctgtagaccccgccgcagaagagccgctcggtggccctgggggccaccgagtcaaacaggtgagtcagcggagcccccgccagtgcctcctcgcaggcccggcgggccctggccaggcgcccctccctggcaaacagggcagagagacgggaatggccgcagccctctcccttgcccgttgaaccgatagcatggctgccagagttccagtagagcagctccgagagctccgccacctccgggggcacggtcgagaagacgctgtagttgtccagctccctggctgccccctcggcctcccgcggccccgggcccgagaccgtgccctcctccggcccggccggcctctccttcccctcagccgccaacaggtgcccgagccccgcctgcctgacctcgttctcaaagagccccgagaccggctccgaattcaccgggaccgccaggtggttgcaggagacgcgggccccctcggccgtggacggggtgccgtggttgggcagcaccatgagctcccccacgcagcgccagcagggcacagaggtgatgtagaggcgcgggtccgggatggggctcacgccccgaaagcggcccagcagctccagggcccggtccaggctctccagccccatgctgtgagacatgcaataaaacacgctattgattctcttcattaagatctcgatgtcgtttattaggtacagacttacatcggccctgtgccccccgtaaaactctacggcgtacgcggcccggaggggagcgggaggcgagatcccgggacccgaccccaccagcagggccgcaacggccctgcgtgagttctccagcaggtgcgccagctccagggacagggcgctgcacgcgcggtcattctgtcgcctcaaacggggaaggaggagggcgtccaactcggccagaaggcccgcgttgcgcgccaccgcctccccgtccaggctccgggggtccagcctggcgcagacgctcagctccacggccagcgcgtacacctggctgtaggccgccgccagcagccccgacatcccgccctggggactctagacctccagacccgggaggaccgcagccagccggcgcttgcgcctgcccccgcctgctccctgccctccgccttccagcaccagcccggccgaggcctgggtggcggcgctgacgggctccgctacgctgataaagttgtcctgggctcccccgggcccacccgccactccttccaaaaagtcacgcgcggcctcggccgtccactctatcccgctggaggcaatggtcgccagtgtttccaggacgctgtccgccagcaccgagaagcggcccaataagtactcggcgtcgtccagagtcagcgaggcgcacgcctcgcccatggcgtccacgagtttgcacactacatcgagcacacaatcttcctcctgtttttgcaatataatggcctccaggcccgccctgatgttctcaatctcataggtggccgctgcctgtgtccggcgcttgatcgtcagccccagggtgggggtggcaaagacaaacttctttctcatggatgagcccccggcctgcttgcgcagcccggcgcccggcgcctgcagcaggttcctgtccacgcctcggcccataaagtagcccaggttcccagcctggaagatttggttgttgccgttgacccccgtgtacttgttaatggtcacgggcagcgtgatgaccgggcgggccttgcagacctggctgaggcagtccgtggccgcgcagaccacggtggtcgcggtgagggaggaggtggcctccgcgtaggccgcggccgactccaccgccctggtgcccagcacgtgggggtagtcgcgggcgggcaccgcctgggtcttgggcaccagcccctggaccaggctgatgtagaactgggtctggccgcaggccttcaggatggcgttgttgagccgctgcttggcgtaggtcaccaggttgcccggcaccacgtctatgacgttgctctcctcgtgggcccgggagcccccgtccacaaacagggccaggtccgagtactcctccgcgctggccccggaggggacgggcaccgagcgccgcctggaaaagttgtgccacaggtagaggctcgagagcttggtgtccgggaagagggtcttgtggtaggtgttgaggaatttcatgtagggcccgttaatgatgtagttttccctcctggcggtggacttgatgaagctgttctggagggcggcgttctcccccgtaaagaccacccggttcttaatcttgatgttcctggggcacagcatcagcaccttggacatgcgcaccggcagccgccgtccgtacgtccgcccctgcagggccgcgtccaggtccggcaggtcacaggtgggctcagcgtgcaccaccttggcctccttggccgtcaggacccccttgtcaatggccaggctcctgaagttggtgcacagcgtctggtagtggcccttgagccactcggggggactctggcccagccccgggttgtcattctcgtagcacatgcagatgggcagggagatgtcctggaggatggtcagcagagagcgataaaacagctgggtaaagacggggcaggcgggctgcgcgaaggggttgcacgagtactgcagcacgtggtagcagctcttgacctggtccttgtaggttatgttgttcttggccatgctgttcataaactggaccacctcggcgtccaccgccgcgtccacgtccctgaacatcttgataaagtcccggggtccctggggctccttctccagcttcccgtcagcgtctatgcccagccgagacagccgctccagcaggttctggttcagctgccagtaggtgtagcggggctcatcgtccggcctctgcgcgtcgtcctccttgtcgataaagttgagaaagttgcccaaaaagtccgtctcgttgtaggagcccgaggcccccgagatcacgtaggggtctctccgctgcgaggacatgacgggcgggaagcggtcccgcagcctgaagaagagcgtgttcaggcacacggccggggcccggccctcgcaaagcgagcacatggggctggtggccgcacccgccacgtagctgccggtctcgggcaccggagtcagcgagctcttctgcccctggcagaactgcaggtaataggcgtagcgggccaggaagttgggagaagaaggaggccgcaaagaccaggtgctccaccgcgtagttgccgggccccgttggtgccagtcaacgtcgggcccgccccaagcccgagaagcagggcccggcgcaaggggtcccaccttcccctcctgcagggtccccagcccctgtgtcatgtaaaaactggttaaaggcgcttctccttgccctgccccggtcgtcttggacacgcccgagaacgtagaggacagaagttggtggcaaatatctgcgtggataccgtggggggccaggctggcattatatcggtgcaacgccgccacgcgggcctcggggccctcgcagtcggcaaacaggggccaggagtcgtagttgaggctggccggcgtctcgtgcgccgcctccagcatggcgggggcgtagctgaccgccagctcgcaggccgcgctgtccacgatcatgacggcccccgagtccgggtggctgatggtcgaggcggggaactccttgagggccgccaccttggccaccttggcctggtcctgcaggctctgcttttccagcagctccaccagcttgcccacctgcctgacgcgcagcgcctgcgccaggcccgtgtacagcgtctcgtgcatgcagctgctgaggtccgagttgtagaactggcgctgctggggcacgccttccgggaacacctccttgtcgtacagcgggaccctaaagctcgcagatggccccacggttacctcctgttttaacgatggaatggccaccaggttccccccgtagaggcgctccttgaatgcctccgtgaccaccaccgcccccaggtaggccgaggggtcgagcccatcggggaagaagtcctccggcttggagctgtccctagagagggcgctgtagccgtcgtacccaaacacctccctggtctcctcgcagagggcctcgaggccggggccctcgaagatggggggcaccatctgggcattgtggaacacgtagacgtccctgtggtaggaggtggcgcgcaggagcccgcagttggggtctggcctcctgtggagggccttgacgttgatgctgaagcccggctccacggtgatgccgcagaggagcggcacagtcaggcacctgtggcccgtgtagccggtgcccagcgtggcgacctccctcagggggtaggtggccagggggtagaagtagatgtagccgcatggccccggctggctctggctgcccagattatcctcgccagtctgtgctgcctgcatgatgcccgcgggtatcgccccgggcctccccgtcccacattaaatgttacactttactcatcaggcaccacccactctttattcgtttacaaagatttcaggaagacgctcaggctggccacggtccatgtcacggggaactggcgtctcagcgcccttggcaggccgcccagtctcgcaaaccagagcctgcgatagagggcgaggtagtgggcggtggcccccatcacgaaggcggcgctctcgtggtcatccaggtagttgcgcaccgcaaacaccaccgtgtagcacagcaccaccctgagccgcgaccagtagtcgtagtggtcatcgtacacggctctcagcacgctgatgatgagctgcatgtgtgcgtgcttgcccccgaggtcggccgcctggcatgccagctccgcgtacattctcctgtgcttcctcagggaggccttcaggagccggcacatgaccagggccggtagaggcaggtccgtctcgtcctgggtgaacacagcgtatagggccctgaacaggaggtagctggtctctgccactttgtcgtccgggggcgagggtgagacccacgcctcgaccggggtcctcacaaacaccgaatcggtagacttggctggctgcatggttccgtctggccagctcacctgccccgggttttatgttaaaatgggcgtggcaaaaccgtatatagcgcgtggcctgcgtgaggtaagtaggtcgccatggccaggcttttcgctcagctgctcctgctcgcgggctccgtcgcctcctgcctggccgtcaccgcctttgtgggtgagcgggccgtcctgagttcctactggaagagggtgagcctagggcccgagatcatggtggaatggttcaaactgggccctggagaggagcagacgctcattgggcgcatgcaccaggatgttatcctgatcgaggggcccttcaggggcttctttgatatgtacaggagcgccaacaccttcttcctcatcctgaccacggccaacatctcccacgacggcagctacctgtgccgcatgcgcctgggggagaccgaggtcacgaagcaggagctgcttagcgtggtgaagcccctgaccctgtctgttcaggccaagaggtctgtattcccagacttctccaccctcaacgtgaggtgcaaggtcaacgcgtacccgcgaccgtatgtccagtggctgatgccagagggcgcggaccccgcgcccaccgtgacacagggtgaagctatgaaggagggggatggcagcctctctgttgtggtgtacctgcacctccacaaaccctggcagctgcctgtgacctgcgttggtaaaaatgaaaaggaggaggtccagggtgtttatgtttctggttatgtgttataataaatgcaactaactgagctggtggccttagtttgctgtgggctggggacagtgggtgcgtgtgtgtgctaattggccgtgtgtgatggggagctcctgggagaaggcttgggggtgggtggcacggtgggagcccgccgtccccgaaagaggcaacgccccgtgggagaccggctccacggtggataggctccctgggagacccggctttccttttgggccacgccccgtgggagaccggctccacggtggataggctccctgggagacccggcttcccttttgggcaacgccccgtgggagaccggctccacggtggataggcaccctgggagacccggcttccctttcgggcaacgccccgtgggagaccggctccacggtggataggcaccctgggagaccggctccacggtggagtggcactgtgggaaaccggttccaccgcgggggtgggtgaatgtcgcgtggcggaagtctttttccgcggttgggaaagtgtgcactggggggccgtcccacggtgggagatttaattcccgtaattggggctcacgagacccgtgtgattttggggtgcaaactccgccccacaggtggctcagattggtgcgatcctggacagctgcggttttaacatcacaagcctgttttcctggggctcgctggggcgtaaatccctcctacctcgtttctcagagagcatttctgcctggtgctgcagcccgggctgcagctatgagcgccttggagatggagccccttggcgcgcccgcgcccgggaccccggtgggccaggatggtggggaaggtagcgggagagaaagctatccatctgttttcagctcgcccccgagctctcctggtccccagggtccggactatgaagaccctcctccagacgatgggaatccacctccttacgacgcggaagacgggggggacggtggtccctatcagcctctgcgtggtcaagaccctaatcagctgtatgcaagactcggaggcggtgggggtaacggcacgctccctccccctccctactctccacagagggaaacatctctgcatctctacgaggaaatacatccgcaaaggtaagtttgccatctagcgatgctttttattaacacacacactcaagttacataattgtctatgccgccacctagcggcctcagtcataccaccccagttcacatctcaatgacaacaagtccaagtttttgccagacacccccagggccgggtccagcgggtgccagcgatagatgcagtcctcgcagtcctgccgcgcgcctcctcccaacgcgtttctgcgtctcaggtgctgggacgttggcatcggagttggcgggcgcccctttcccgggtcccccttctcctcggcctcctctttgggccttttggacattcttctgagggaacggcgaagacgagagcagaggtccgacaggtgtctaccgggcgccatggagtctcttagattagtagcaggcagacgagaccaaacagggcgcagagcacgaccaggcagattctcaggttcttgagctcttctttgaccgggcagtggcttggcttctgcccctcagaggagccgggcaggccgtaggcagggtagcgctgctccaggaaccccgggttcagaaagaagaccatctcgggagtgaggagcagactggctcctcgggtttatatacaaccagaacaaggaaagaaggccagaggaatgtggaaagatgagcaaggaggcaggtatcgggttagcgatgggtcagaaaataggtgtggggtggaaacaaagtgccaaagtgtcaaggtgattgctcactcggggtgcccggtcttgcaggttctgagtgggtgttctgggcctagtcacggtttctggcagtggatgcatatgttttgcctagtggcattagttgtgttgcaatttgttttttgggagaagggcgtgggggaagtcactggcatgggcaatgcccagttgaggcctctccccggaaggcgtggggcaggatttttgttgggaatgggacggggatatggaggaggaagtagttgtgcaggcgcgtgggccttggtggggccacatctcgcatgccttcctttagttgtgtaaggtgtgtgggccttagtggggccaattctagcaggccatcctctagttagtatgcagcctgggccttggcgggccaattttagcatgcctttctctacctagttttgcaggagcttgggctttgccgggccaattccagcatgccatcctctgcttagttgtgcaggatgaagcgtgggccttgttgggggccaattttcgcatgccatactccaccccctgttatctgatagaatgtgagtaaggcctgtcaaaatgcaccagagttgcctaataggagcttgggtcagataaggtgaatagctaaacaatcccgggggtgaagggaggggcatcaatggttaccgtcagccgtcctgattccgagaaggaaaagttcacgagtggccacctcagtcatagtagctgatctggattggatctgccggatcacccgggtcaccttccggaccttgtgggccttgattggatggaggactatccgggccattgttaccaccatcagtggggcctggtgggccacctccctcgttgctttcaggctgctgattatgaggtccgccggggccactgccgttgctgttgtcggggttttgtggcccgttgcccccgttaccacggtcatttaggtgaacactgttgccgccgccgccaagggtcaggagcatccaaggaccacccggtcctgtttcttgcagtgggccgtggttgttgttggggccccatggaccccagggcccacatggccctgggccaaagggaccccatggcccaagaggaccggtaccaaacggaccccagggccacgaaggaccttgagtaaacggaccagctggaccatagggacccaaaccatctccaccgtcagaagcttgcactgggtgtggtagatgatcggggtcgccagacggaccctgagaaacatagggcccgaatggacccctgtctccaccaccttcaccgtcttcaccatcgcccccatctcctccgtcaccactgtttccaccgtcagttgcttggatggggtgagggagatggtctggatccccaccgcctccaccgttgcctccatttcctccgtctccatctcccccatctccctgtcctccgttaccaccgtcagttgcttggatcgggtaagggagaggatctgggccctcgttgcctccatctccttctcctccattgccaccatcagttgcttggatcgggtaagggagatggtctgggccctcattgcctccgtctccattgcctccgtctccattgcctccgtctccattgcctccgtttccattgcctccgtctcctacattgcctccatctccattgcctccatctccattgcctccatctctattgcctccgtctccttcattgcctccatttccaccgttttgggaaacatagggacctagcgggtcaccctggttactgtttttatggtgatggtggtgatggtggtgatggtgggtttttgggtcctcgctaatcagaagcacccgcggctggtgctcgtccccctgtcccccgccatggccatgtccctggtgattatactcctcttccaagttagctagagcctctggaattgcacggttgaaatcatggacgtaaatccaaatgaggatggccaagaatagcagaagccaatagagatcgaccaggaccgtaaaccaggccaggtgcagcaggacagcgataatcaggaagactatggccagtaggaaggccaggcagaaggccaggattgtccagaaggaggcaccgtattctcggagcagccacatgaggtagaaccagacagcgagcgctgcagaaatgagaggacagggttagattggaggagattcctgtggcccaagtggtcatcggcggtgggaaatgcagagcttacctaagagaatgcagaggcagaggatcacgagtcccacgaagagggtctgtccagtgagcgtcaggaggttggacaccaggatcacaactgcagagaagagagagcgggggtcagggagggggatgggaatatgggtaggtggttgcaggaagggaaggggggtgcttactcactcataattaagatgatgcacaagagtcccagggggcacagcagccgtctcttgaataacatgatgatgatgatgatgatgatcaggaccactgcaaaggagctgaggactgtgctggcagtctgagttaggtcagacatgatgataaaaaaccagaccagaattgccacgaggaggagcagggatccaaagagtagggatgcatgcggttgtgggcatcgggggtgcctgtggcccccgcccctgcccctattcccctccatgctgctggtgtgctgctggtgtgctggaggagcctgacagtctcagaatctaggcagggaggagacggagggggagggactctgggctgtttgaatgcaccaatgggagagccgtgcgtggggattgcaggtggtctcggggggagtctgatagtgcaaaaacaggctggcagagactgagaatgagtgtttaggggcggtgactggggacatttaaaaaaggggaagttgaaagcacgggtctaggctcagagagcggatgggggtgtgtagattcgcgggaattcgcagccgctgtgggaaagtagaataggcggagcgtgaagaacaacctgtgaattctgcagcatgtgaaccgcgattataggaaatgggcggtgtaggcacgagagaattctcagggaagtagcgtggtagctagaatcgcagcgtggtgccagtcgggcggctgtgccaagcggcagggtccttggcaggttggtaggggtcttcaggcagccgaaagtgttatgcagatgtgtgcactgctcagagaggctgacagcggatatggtagtctcagaatgagtggccgcggacgccagagcgagccgggaagggggagcggtgtggcctggcaggggggagacggggcaatgggaggggggtgtgaggggttgagccaatgggcatgggtttcggacgggaccagcagtggcgaggggaccaggaggggagtttgaaaagggcaatggtgttgttttccgtgcctgtgcttttgtcggggacattggggggatgttcttaggaagcccggcgggaggggggagcggagggcgaggaggagcgtgagtgaaaaacagaggatgagctgtgtgaagcggtgtgcagccagggggagtgctgctgcatgaggaggggaaatagaggacggggcagtggctgggcgcggttgaaggtgcacgtggtggctggggcaaagcaggagtgtcccaaaagtggtccagggcgaccccccttacccgccggctccaaaaaagtgaggggggcgtggccatcccccgcgtgccccccccccccccccccccccgcgtagaaagtgggcactgtgccagggccttcgggacgggaggccggcgcgcgcccggggtcccggggggcggcgcgcggccttcccccgtctgctcccgcggccagtccctggatgtcggggagggcccggggccgcgcgtggggatggccggcgggaggggccggcgcctgcagggggggccggcggggcgtcccgtcgtcacgctcggctgcacgcgcggcggcgccggccgggggctgagggggctcccgagggcggggccggggcctggcgggggccagcgcggggtcccggggcggggggtcgggcgggcatgctccggggtaacgagaggagagaggagggtgtgcctggcgggggagagggggcagggctggcgccgggccgcgcccccggcccctctcctgggaggccacgtgtggaggcccgcggagaggccgtgtgtggaggccgggcctcccctgggggcctcgggggcggaggggggggtcccgcggggcccggcgcgtgccgggggacccgggggcgtgtcccgcgacccgaggggcgagcggggggcttccccggggcccgagcgcgcgtcgggtggggcctgagggggcgcggcgattgtcgccgccctccctctgctcccgttggcggggagaatgacagctgggcgtggcgggcgcgcgtggcccgcccccgggtcttcccgggctccccctcacgggtgggcgctcagatcggcgcgggggagccccggggcggcccggggaccctcgcgggggcaccggcgcgtggagaagacccttctccgacccagccgagcgtgacgaagcccctctcctaaatcacccttgcgtgacgaagcccctctcctaaatcacccttgcgtgacgaagcccctctccgaaatcacccttgcgtgacgaagcccctctccgaaatcaccagaaagtgggcactgtgccagggccttcgggacgggaggccggcgcgcgcccggggtcccggggggcggcgcgcggccttcccccgtctgctcccgcggccagtccctggatgtcggggagggcccggggccgcgcgtggggatggccggcgggaggggccggcgcctgcagggggggccggcggggcgtcccgtcgtcacgctcggctgcacgcgcggcggcgccggccgggggctgagggggctcccgagggcggggccggggcctggcgggggccagcgcggggtcccggggcggggggtcgggcgggcatgctccggggtaacgagaggagagaggagggtgtgcctggcgggggagagggggcagggctggcgccgggccgcgcccccggcccctctcctgggaggccacgtgtggaggcccgcggagaggccgtgtgtggaggccgggcctcccctgggggcctcgggggcggaggggggggtcccgcggggcccggcgcgtgccgggggacccgggggcgtgtcccgcgacccgaggggcgagcggggggcttccccggggcccgagcgcgcgtcgggtggggcctgagggggcgcggcgattgtcgccgccctccctctgctcccgttggcggggagaatgacagctgggcgtggcgggcgcgcgtggcccgcccccgggtcttcccgggctccccctcacgggtgggcgctcagatcggcgcgggggagccccggggcggcccggggaccctcgcgggggcaccggcgcgtggagaagacccttctccgacccagccgagcgtgacgaagcccctctcctaaatcacccttgcgtgacgaagcccctctcctaaatcacccttgcgtgacgaagcccctctccgaaatcacccttgcgtgacgaagcccctctccgaaatcaccagaaagtgggcactgtgccagggccttcgggacgggaggccggcgcgcgcccggggtcccggggggcggcgcgcggccttcccccgtctgctcccgcggccagtccctggatgtcggggagggcccggggccgcgcgtggggatggccggcgggaggggccggcgcctgcagggggggccggcggggcgtcccgtcgtcacgctcggctgcacgcgcggcggcgccggccgggggctgagggggctcccgagggcggggccggggcctggcgggggccagcgcggggtcccggggcggggggtcgggcgggcatgctccggggtaacgagaggagagaggagggtgtgcctggcgggggagagggggcagggctggcgccgggccgcgcccccggcccctctcctgggaggccacgtgtggaggcccgcggagaggccgtgtgtggaggccgggcctcccctgggggcctcgggggcggaggggggggtcccgcggggcccggcgcgtgccgggggacccgggggcgtgtcccgcgacccgaggggcgagcggggggcttccccggggcccgagcgcgcgtcgggtggggcctgagggggcgcggcgattgtcgccgccctccctctgctcccgttggcggggagaatgacagctgggcgtggcgggcgcgcgtggcccgcccccgggtcttcccgggctccccctcacgggtgggcgctcagatcggcgcgggggagccccggggcggcccggggaccctcgcgggggcaccggcgcgtggagaagacccttctccgacccagccgagcgtgacgaagcccctctcctaaatcacccttgcgtgacgaagcccctctcctaaatcacccttgcgtgacgaagcccctctccgaaatcacccttgcgtgacgaagcccctctccgaaatcaccagaaagtgggcactgtgccagggccttcgggacgggaggccggcgcgcgcccggggtcccggggggcggcgcgcggccttcccccgtctgctcccgcggccagtccctggatgtcggggagggcccggggccgcgcgtggggatggccggcgggaggggccggcgccgcagggggggccggcggggcgtcccgtcgtcacgctcggctgcacgcgcggcggcgccggccgggggctgagggggctcccgagggcggggccggggcctggcgggggccagcgcggggtcccggggcggggggtcgggcgggcatgctccggggtaacgagaggagagaggagggtgtgcctggcgggggagagggggcagggctggcgccgggccgcgcccccggcccctctcctgggaggccacgtgtggaggcccgcggagaggccgtgtgtggaggccgggcctcccctgggggcctcgggggcggaggggggggtcccgcggggcccggcgcgtgccgggggacccgggggcgtgtcccgcgacccgaggggcgagcggggggcttccccggacccccagcgcgcggcgggcggggcctgagggggcgcggcgattgtcgccgccctccctctgctcccgttggccgggagaatgacagctgggcgtggcgagcgcgccgggcccgcccccgggtcttcccgggctccccctcacgggtgggcgcgcagatcggcgcgggggaggccaggggcgccccggggaccgtcgcgggggcaccggcgcgtagagaagacccttttccgacccagccgagcgtgacgaagcccctctccgaaatcacccttgcgtgacgaagcccctctccgaaatcacccttgcgtgacgaagcccctccccgaaatcacccttgcgtgacgaagcccctctccgaaatcacccttgcgtaacgaagcccctctccgaaatcacccttgcgtgacgaagcccc

(19) United States
(12) Patent Application Publication
(10) Pub. No.: US 2004/0214161 A1
(43) Pub. Date: Oct. 28, 2004

(54) Detection of Epstein Barr virus

(76) Inventor: Melvyn Smith, London (GB)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/612,869

(22) Filed: Jul. 2, 2003

(30) Foreign Application Priority Data
Apr. 24, 2003 (GB) 0309311.9

(51) Int. Cl. C07H 21/04; C07H 21/00; C07H 21/02; C12Q 1/68; C12Q 1/70

(52) U.S. Cl. 435/5; 435/6; 536/23.72; 536/24.32; 536/24.33; 536/25.32

(57) ABSTRACT

The present invention provides a method for detection of Epstein Barr virus nucleic acid in a sample, comprising contacting said sample with a probe wherein the probe binds to a target region defined by SEQ. ID NO. 1 or its homologue, or a complementary strand thereof, which binding provides a detectable signal, and detecting said signal.

### CROSS REFERENCES TO RELATED APPLICATIONS

This application claims priority from UK Patent Application No. 0309311.9, filed Apr. 24, 2003, which is incorporated herein by reference in its entirety.

### BACKGROUND OF THE INVENTION

The present invention relates to a method for detection of Epstein Barr virus nucleic acid, preferably in an isolated sample, and to reagents and kits therefor.

Epstein-Barr virus (EBV) is a ubiquitous human herpes virus. It is transmitted principally by saliva, infecting oropharyngeal epithelial cells that are permissive to viral replication. A primary EBV infection tends to have a biphasic distribution, often occurring in asymptomatic infants from 1-6 years of age or in young adults from around 14-20 years of age who develop infectious mononucleosis (glandular fever). EBV infection is also associated with a spectrum of lymphoid malignant syndromes such as Burkitt's lymphoma, Hodgkin's disease and post-transplant lymphoproliferative disorders.

Post-transplant lymphoproliferative disorder is a result of neoplastic proliferation of B cells in immunosuppressed individuals. A primary infection by EBV or reactivation of EBV in post-transplantation recipients may be caused by transmission from the donor organ or by exposure of EBV to environmental stimuli. Post-transplant lymphoproliferative disorder in solid organ transplant patients is associated with EBV infected host B cells, whereas post-transplant lymphoproliferative disorder in bone marrow transplant recipients tends to arise from donor cells (Hsieh et al. 1999). Several single centre studies have reported that primary EBV infection in this clinical setting carries a high risk of developing post-transplant lymphoproliferative disorder. EBV susceptible individuals experienced a 10-76 fold greater incidence of post-transplant lymphoproliferative disorder compared to those with previous EBV exposure (Cockfield et al. 1993, Ellis et al. 1999, Swinnen et al. 1990, Walker et al. 1995a, Walker et al. 1995b).

Early identification and diagnosis of EBV infection is critical since reduction of immunosuppression and use of antiviral agents may affect disease progression.

EBV isolation is not possible in standard cell cultures, although it is possible using rare cell lines that are difficult to grow and maintain.

Tests have been developed to detect non-specific heterophile antibodies, so-called because they can react with antigens from unrelated species. Heterophile antibody tests are, however, limited by their nonspecificity and insensitivity, particularly in young children.

Currently used tests include several serological assays that detect EBV specific antibodies to the viral capsid antigen (VCA), the early antigen (EA) and the EBV nuclear antigen (EBNA). Antibodies to these specific antigens are detected by indirect immunofluorescence assays, enzyme immunoassays, and immunoblot techniques. However, these serological tests suffer from a number of problems. For example, they are cumbersome and slow, do not correlate well with the viral load and may be sub-optimal in an immunocompromised host where development of antibody is often late and may even be absent (Fischer et al. 1991, Henle et al. 1981).

Advances in nucleic acid techniques and in particular polymerase chain reaction (PCR) based methods have enabled sensitive and specific detection of many infectious agents in diagnostic microbiology. This is of particular relevance in cases where the number of infectious organisms to be detected is low. However, application of PCR requires both knowledge of the nucleotide sequence of the organism and careful selection of a target sequence for amplification. Each of these requirements is beset with difficulties, for example, suitable sequence information is not always available and is time-consuming to prepare. It is also particularly difficult to identify good potential target sequences for detection purposes.

Recent expansion in molecular biology techniques has led to the development of several block-based quantitative PCR (Q-PCR) assays for detecting EBV DNA as a prognostic marker in blood samples. These block-based methods suffer from the disadvantages of being time consuming and lacking in specificity.

A recent development of the standard PCR assay is the emergence of real-time detection methods such as the Applied Biosystems TaqMan assay, which employs a sequence-specific dual-labelled fluorogenic probe for quantification of the amplified product. The probe comprises reporter and quencher fluorophores, which are separated by cleavage of the probe, resulting in a change in the fluorescent signal, which may be detected. Cleavage of the probe may occur in a real-time PCR assay when an extending polymerase with 5′ to 3′ exonuclease activity encounters a probe molecule bound to the target nucleic acid site.

Two reports have recently described the development of real time Q-PCR TaqMan assays for the detection of EBV DNA in similar patient cohorts (Kimura et al. 1999, Neisters et al. 2000). These papers describe two-step Taqman PCR programs with combined annealing/extension times of up to 60 seconds. The total duration of a TaqMan run can, however, be lengthy, and this method does not allow for melt-curve analysis—an analysis that can assist in interpreting questionable positive results.

Alternative, real time Q-PCR probe systems for detecting specific nucleic acid sequences include Molecular Beacons assays and Scorpions assays.

A yet alternative, real time PCR system uses LightCycler technology (Roche Diagnostics GmBH, Germany). The Roche LightCycler system typically uses a pair of fluorescently labelled oligonucleotide probes, one labelled at its 3′ end with a donor fluorophore and the other labelled at its 5′ end with an acceptor fluorophore. Hybridisation of the probes to their specific target nucleic acid sequences brings the donor and acceptor fluorophores into close proximity, enabling a transfer of energy by Fluorescence Resonance Energy Transfer (FRET). This results in a change in the fluorescent signal, which can be detected fluorometrically. Once the PCR is complete, samples are subjected to an incremental increase in temperature, causing the sequence specific probe to dissociate from the PCR product and resulting in a dramatic fall in fluorescence.

Whilst the Roche system appears to be superior to previously available EBV detection systems, there is still a need for an improved (eg. more sensitive) EBV detection system.

In summary, there is a need in the art to develop an improved method for detection of Epstein Barr virus in a sample, that overcomes or at least ameliorates one or more of the above-described technical problems in the art.

In addition, there is a need to develop a real time quantitative EBV assay to investigate the natural history of EBV infection in immunosuppressed hosts, to identify those at greatest risk of developing lymphoproliferative disorders and to monitor responses to therapy.

The above needs are fulfilled by the present invention, which solves one or more of the above defined technical problems.

### SUMMARY OF THE INVENTION

Accordingly, the present invention provides a method for detection of Epstein Barr virus nucleic acid in a sample, comprising contacting said sample with a probe wherein the probe binds to a target region defined by SEQ. ID NO. 1 or its homologue, or a complementary strand thereof, which binding provides a detectable signal, and detecting said signal.

A sample may be, for instance, a food, sewerage or clinical sample. A particular application of the method is for detection of Epstein Barr virus in a clinical sample. Clinical samples may include blood, plasma, cerebrospinal fluid, lymph or other body fluid samples.

SEQ. ID NO. 1 represents the BZLF 1 gene (GenBank Accession No. >gi9625578:102210-103155). The BZLF 1 gene codes for the ZEBRA protein.

### BRIEF DESCRIPTION OF THE DRAWINGS

FIG. 1 shows the melt curve analysis of the BZLF1 probe (70° C.) in serial log dilutions of pGEM-BZLF1 standards from 109 to 1 copy per reaction.

FIG. 2 shows the PCR amplification curve of pGEM-BZLF1 serial log dilutions ranging from 10⁹ to 1 copy per reaction and a water negative control (indicated by the arrow).

FIG. 3 shows a standard curve representing a serial log dilution of pGEM-BZLF1 standards.

FIG. 4 shows the melt curve analysis of the 10⁸ pGEM-BZLF1 external standard at 70° C. (indicated by the arrow).

FIG. 5 shows results of a comparative assay carried out using the method of the invention at two different test sites (Birmingham and CPHL), the known Roche EBV DNA real time PCR assay, and the known Leeds TaqMan assay.

FIG. 6 shows conventional sequence alignment methodology as applied to the present invention.

FIG. 7 shows the region of the BZLF 1 gene sequence to which, in a specific embodiment, the forward and reverse primers and the probe bind.

### DETAILED DESCRIPTION OF THE INVENTION

An advantage of the present invention is that the target nucleic acid sequence does not encode a surface protein. This is beneficial because surface proteins are susceptible to immune selection pressure, leading to nucleotide sequence variation, which is undesirable for diagnostic purposes.

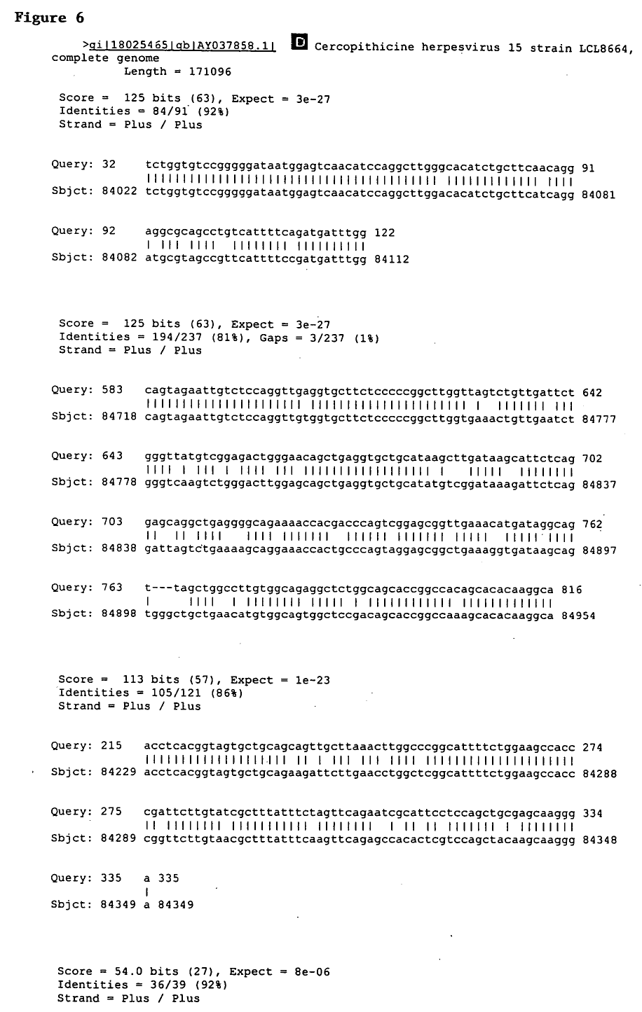

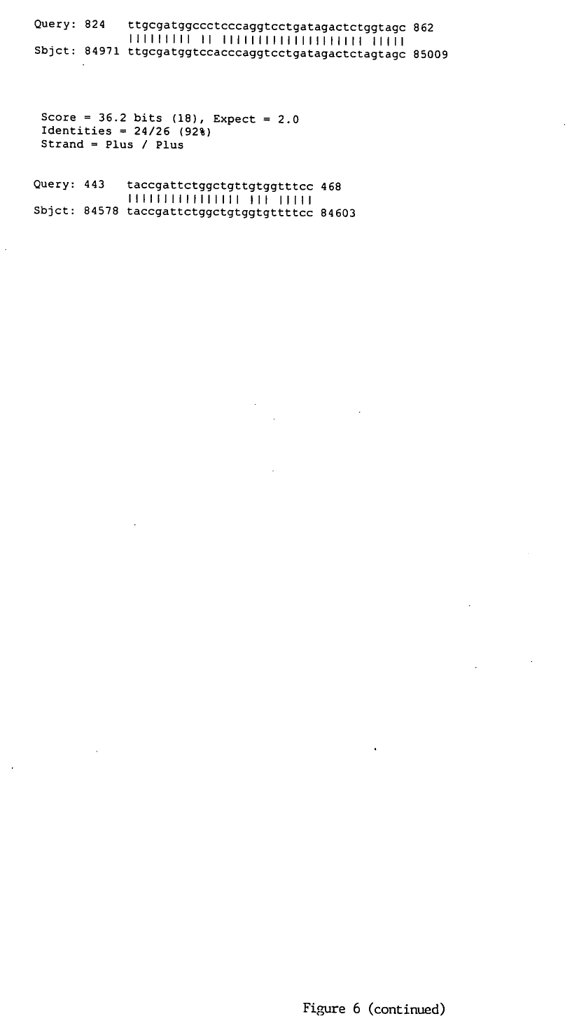

The present invention also concerns detection of homologues of SEQ. ID. NO. 1, which exist in other viruses, in particular in other herpes viruses. In this regard, the term “homologue” embraces the sequence of the gene encoding the ZEBRA protein of any virus. Homologous sequences may be identified by sequence alignments using conventional software, for example the Bioedit™package, available free online, and the Sequencher™package, provided by Sequencher Gene Codes Corporation, 640 Avis Drive Suite 310, Ann Arbor Mich. 48108. An example of this alignment methodology as applied to the present invention is demonstrated in FIG. 6.

By way of example, a homologue of the Epstein Barr virus BZLF 1 gene sequence (SEQ. ID. NO. 1) exists in African Green Monkey herpes virus (SEQ. ID. NO. 8).

In the present invention, a complementary strand means the non-coding (anti-sense) nucleic acid strand, which may bind via complementary base-pairing to a coding strand. In the present invention, the coding strand is that represented by SEQ. ID. NO. 1 or its homologue, such as SEQ. ID. NO. 8.

One embodiment of the present invention further comprises the step of amplifying Epstein Barr virus nucleic acid prior to detection of signal. Amplification may be carried out by methods known in the art, preferably by PCR and most preferably by quantitative and/or real-time PCR. For example, a TaqMan system may be used. The assay was originally developed on a block-based PCR machine and good results were achieved in this type of assay. In a specific embodiment of the invention, the amplification step is carried out using a LightCycler system.

The advantages of the LightCycler embodiment of the present invention include the rapid thermal cycling times compared with conventional PCR machines, including TaqMan assays; rapid sequence-specific detection via probe/melt curve analysis; and melt curve analysis carried out post-PCR in the same, unopened reaction vessels, thus eliminating contamination.

Amplification of Epstein Barr viral nucleic acid is preferably carried out using a pair of sequence specific primers, which bind to a target site within the Epstein Barr viral nucleic acid and are extended by a polymerase, resulting in nucleic acid synthesis. Primers of the present invention are designed to bind to the target gene sequence based on the selection of desired parameters. Conventional software, such as Primer Express (Applied Biosystems), may assist in the selection process.

Preferred target sequences (and thus probes) include a high GC content, for example, at least 30%, preferably at least 40%, more preferably at least 50%. In this regard, it is preferred that the binding conditions are such that a high level of specificity is provided—as confirmed by BLAST database analysis. The annealing temperature (Tm) of the primers is preferably in excess of 50° C., more preferably in excess of 55° C. and most preferably about 60° C. The primers of the present invention are preferably screened to minimise self-complementarity and dimer formation (primer-to-primer binding).

The primer pair comprises forward and reverse oligonucleotide primers. A forward primer is one that binds to the complementary, non-coding (anti-sense) strand of the target Epstein Barr virus nucleic acid, and a reverse primer is one that binds to the coding (sense) strand of the target Epstein Barr virus nucleic acid.

The forward and reverse oligonucleotide primers are typically 1 to 50 nucleotides long, preferably 10 to 30 nucleotides long, more preferably 15 to 25 nucleotides long. It is an advantage to use short primers (eg. less than 50, preferably less than 40 nucleotides), as this enables faster annealing to target nucleic acid and hence faster overall duration of the assay.

In one embodiment of the invention, the amplifying step is carried out using a pair of primers, comprising forward and reverse oligonucleotide primers, the forward primer binding to a target site between nucleic acid residues 1-200, preferably 1-100, of the complementary strand of SEQ. ID NO. 1 or its homologue, and the reverse primer binding to a target site between nucleic acid residues 1-500, preferably 100-300, of SEQ. ID NO. 1 or its homologue.

It should be noted that the above numbering system applied to the nucleic acid residues of the complementary strand of SEQ. ID. NO. 1 is based on the numbering of the nucleic acids of SEQ. ID. NO. 1 to which they are complementary.

Particularly good results have been obtained using a forward oligonucleotide primer of SEQ. ID NO. 4 or 5 and a reverse oligonucleotide primer of SEQ. ID NO. 6 or 7, as shown in the table below.

| | |
|---|---|
| SEQ. ID NO. | SEQUENCE |

| | |
|---|---|
| 4 | 5′ -GCA CAT CTG CTT CAA CAG-3′ |
| 5 | 5′ -GAA CAG CTG CTT CAA CAG-3′ |
| 6 | 5′ -CGT GAG GTC AGT ATA TAC-3′ |
| 7 | 5′ -CGT AAG TTC AGT ATA TAC-3′ |

It will, however, be appreciated that variants may be employed, which differ from the above-mentioned primer sequences by one or more nucleotides. In this regard, conservative substitutions are preferred. It is also preferred that primers do not differ from the above-mentioned primers at more than 5 nucleotide positions. It is further preferred that any sequence variation occurs within the first 10 nucleotide positions from the 5′ end of the above-mentioned primers.

Suitable probes for use in the present invention are ligands that bind specifically to Epstein Barr virus nucleic acid. Such ligands may be oligonucleotide ligands or protein ligands, for example, antibodies.

It is preferred that the probes are oligonucleotide probes. Probes are designed to bind to the target gene sequence based on a selection of desired parameters. Conventional software may assist (as described above for primer selection). Again, a high GC content for target sequence (and thus probe) is preferred. In this regard, a preferred probe has a GC content greater than 30%, more preferably greater than 40% and most preferably greater than 50%. It is preferred that the binding conditions are such that a high level of specificity is provided—as confirmed by BLAST database analysis. In this regard, the annealing temperature (Tm) of the probe is preferably above 60° C., more preferably above 65° C. and most preferably about 70° C. Most preferably, the probe Tm is slightly higher (preferably at least 5° C. higher, more preferably at least 10° C. higher) than that of the primers.

It is preferred to screen potential probes to minimise self-complementarity and dimer formation (probe-probe binding). In addition, preferred probes of the present invention are selected so as to have minimal homology with human DNA. The selection process may involve comparing a candidate probe sequence with human DNA and rejecting the probe if the homology is greater than 50%. The aim of this selection process is to reduce annealing of probe to contaminating human DNA sequences and hence allow improved specificity of the assay.

In a typical method of the present invention, the oligonucleotide probe is 1-50 nucleotides long, preferably 10-30 nucleotides long, most preferably 15-25 nucleotides long. It is an advantage to use short probes (eg. less than 50 nucleotides, preferably less than 40 nucleotides), as this enables faster annealing to target Epstein Barr virus nucleic acid and hence shorter overall assay duration.

In a method of the invention, the probe binds to a target region defined by SEQ. ID. NO. 1 or its homologue or a complementary strand thereof. In one embodiment of the invention, the probe binds to a target site between nucleic acid residues 1-500, preferably 1-300, of SEQ ID NO. 1 or its homologue or a complementary strand thereof.

It will be appreciated that the above numbering system applied to the nucleic acid residues of the complementary strand of SEQ. ID. NO. 1 is based on the numbering of the nucleic acids of SEQ. ID. NO. 1 to which they are complementary.

Good results have been obtained using a probe of sequence SEQ ID. NO. 2 or 3, as shown below.

| | |
|---|---|
| SEQ. ID NO. | SEQUENCE |

| | |
|---|---|
| 2 | 5′-GCC GCC TGG GCA TTC GTG TTA-3′ |
| 3 | 5′-GCC GTC CGG GCA TTC GTG TTA-3′ |

It will, however, be appreciated that variants may be employed, which differ from the above-mentioned probe sequences by one or more nucleotides. In this regard, conservative substitutions are preferred. It is also preferred that probes do not differ from the above-mentioned probes at more than 5 nucleotide positions. It is also preferred that any sequence variation occurs within the first 10 nucleotide positions from the 5′ end of the above-mentioned primers.

Binding of probe to Epstein Barr virus nucleic acid provides a detectable signal, which may be detected by known means. A detectable signal may be a radioactive signal but is preferably a fluorescent signal, most preferably a change in fluorescence.

In one embodiment, the probe of the invention is labelled and the assay comprises detecting the label and correlating presence of label with presence of Epstein Barr virus nucleic acid. The label may be a radio-label but is preferably non-radioactive, such as digoxygenin, fluorescein or fluorescein-isothiocyanate (FITC). The label may be detected directly, such as by exposure to photographic or X-ray film, or indirectly, for example, in a two-phase system. An example of indirect label detection is binding of an antibody to the label. In another example, the probe is labelled with biotin and is detected using streptavidin bound to a detectable molecule or to an enzyme, which generates a detectable signal.

In a preferred embodiment, said probe is fluorescently labelled. The fluorescent label may comprise one or more fluorophores. In one embodiment, the probe comprises reporter and quencher fluorophores. A reporter may be bound at the 5′ end of the probe and a quencher may be bound at the 3′ end of the probe. Alternatively, a reporter may be bound at the 3′ end of the probe and a quencher may be bound at the 5′ end of the probe. Cleavage of the probe separates the reporter and quencher fluorophores, resulting in a change in the fluorescent signal, which may be detected. Cleavage of the probe may occur in a real-time PCR assay when an extending polymerase with 5′ to 3′ exonuclease activity encounters a probe molecule bound to the target nucleic acid site.

In a particular embodiment of the present invention, the TaqMan system for amplifying and detecting a target nucleic acid sequence is employed. For optimal performance of the Taqman assay the length of the amplified product is less than 200, preferably less than 150 base pairs, and the probe preferably has a melting temperature at least 5° C. higher, preferably at least 10° C. higher than the primers. Typically this results in the probe being longer than the primers (eg. by at least 5 nucleotides, preferably by at least 10 nucleotides).

It is also an option for one or both of the forward and reverse primers to be labelled, preferably fluorescently labelled. In a preferred embodiment of the present invention, both the probe and one or both of the forward and reverse primers are fluorescently labelled.

In a preferred embodiment of the present invention, amplification is carried out using a LightCycler system, such as the Roche LightCycler system.

In a particularly preferred embodiment of the present invention, a single probe is used and one of the primers and the probe are fluorescently labelled. For example, the primer may be labelled with a donor fluorophore, and the probe with an acceptor fluorophore. Alternatively, the probe may be labelled with a donor fluorophore and the primer with an acceptor fluorophore. In more detail, a preferred embodiment of the present invention employs a labelled primer/probe combination. As the probe binds to the amplified target sequence, the donor fluorophore excites the incorporated acceptor fluorophore by the principle of Fluorescence Resonance Energy Transfer (FRET). The resultant change in fluorescence can be detected fluorometrically. Once the PCR is complete, samples are subjected to an incremental increase in temperature, causing the sequence specific probe to dissociate from the PCR product and resulting in a dramatic fall in fluorescence.

In one embodiment of the present invention, of the primers (preferably the reverse primer) is labelled with an acceptor fluorophore such as LCRed 640 and the probe is labelled with a donor fluorophore such as fluorescein.

When the probes and primers of the invention are employed, this system operates with annealing and extension times of 5 and 6 seconds respectively, producing quantitative results in less than 1 hour. The faster turnaround time of the LightCycler in comparison to the TaqMan system offers advantages with respect to the number of runs that can be performed per day.

The EBV BZLF1 PCR assay of the invention is rapid, specific and is sensitive enough to detect a single copy of EBV DNA. Intra- and inter-assay variability studies carried out using the external quantitative standards and DNA extracted from whole blood and plasma samples were shown to be within a 0.5 log₁₀ range.

The present invention therefore enables monitoring of EBV viral load in a variety of clinical situations, and can be used for long-term studies of transplant recipients to further define the role of EBV load measurements in a clinical setting.

Accordingly, the invention also provides a method of quantifying EBV viral load in a first isolated sample, comprising (i) contacting said first sample with a probe wherein the probe binds to a target region defined by SEQ. ID NO. 1 or its homologue or a complementary strand thereof, which binding provides a detectable signal, and detecting said signal and comparing the results obtained in step (i) with results obtained using a second, control sample having a known EBV viral load and thereby quantifying EBV viral load in the first isolated sample.

The above quantification method preferably employs a nucleic acid amplification step, which is a time-dependent process. Thus, by employing a defined nucleic acid concentration (or range of nucleic acid concentrations) in a second control sample (or second control samples) and amplifying said first and second samples through an identical number of amplification cycles, it is possible to quantify the EBV viral load in a second sample.

Alternatively, the first sample may be compared to a serial dilution of known nucleic acid controls (eg. herring sperm carrier DNA at a log dilution of one or more of 10⁹, 10⁸, 10⁷, 10⁶, 10⁵, 10⁴, 10³, 10² or 1 ng DNA/□l) having a measurable signal, which signal is proportional to the amount of nucleic acid present.

The invention also provides an in vitro method of monitoring drug efficacy for alleviating EBV infection or an EBV induced medical condition, comprising (i) contacting in vitro a first sample with a probe wherein the probe binds to a target region defined by SEQ. ID NO. 1 or its homologue or a complementary strand thereof, which binding provides a detectable signal, and detecting said signal, wherein said first sample has been isolated from a patient and (ii) contacting in vitro a second sample with a probe wherein the probe binds to a target region defined by SEQ. ID NO. 1 or its homologue or a complementary strand thereof, which binding provides a detectable signal, and detecting said signal, wherein said second sample has been isolated from a patient after commencement of drug therapy and comparing the results from (i) and (ii) and thereby confirming the efficacy of said drug.

It will be understood that preferred embodiments (eg. preferred EBV detection parameters, and preferred primer/probe embodiments) described hereinbefore apply equally to the above two methods for quantifying viral load and monitoring drug efficacy. The invention also provides reagents for use in the above-described methods, and hence provides a probe and forward and reverse primers for use in accordance with the invention.

The invention also provides use of a probe according to the invention in the manufacture of a composition for detecting Epstein Barr virus nucleic acid. The invention further provides use of a forward primer or a reverse primer according to the invention, in the manufacture of a composition for detecting Epstein Barr virus nucleic acid. The invention yet further provides use of a pair of forward and reverse primers according to the invention in the manufacture of a composition for detecting Epstein Barr virus nucleic acid.

Also provided by the present invention is a kit for detection of Epstein Barr virus nucleic acid comprising a probe according to the invention and a pair of primers according to the invention. The present invention also provides a kit for detection of Epstein Barr virus nucleic acid comprising a probe according to the invention and a forward primer or a reverse primer according to the invention.

A further aspect of the present invention relates to the preparation of a DNA array comprising the BZLF1 gene (or a preferred target sequence thereof as described above) as a probe, and to the use thereof in diagnostic analysis (eg. EBV detection, quantification of viral load, and monitoring patient response to drug therapy). The DNA array may further comprise different EBV genes (or a target sequence thereof) as probes, and/or other pathogen probes, thereby allowing simultaneous diagnostic analysis of EBV via multiple targets and/or simultaneous diagnostic analysis of other pathogens. The basic preparation and use of such a DNA array is described in more detail immediately after Table 3 in the present specification.

Preferred embodiments of the invention are now described with reference to the figures.

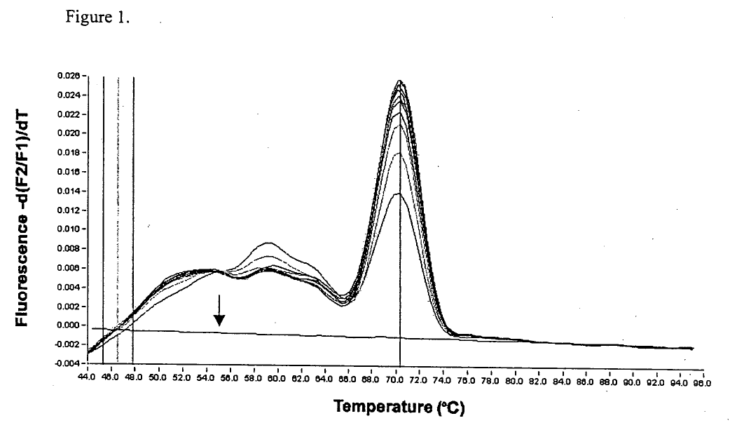

FIG. 1 shows the melt curve analysis of the BZLF1 probe (70° C.) in serial log dilutions of pGEM-BZLF1 standards from 10⁹ to 1 copy per reaction. No Tm is present in the water negative control (indicated by the arrow).

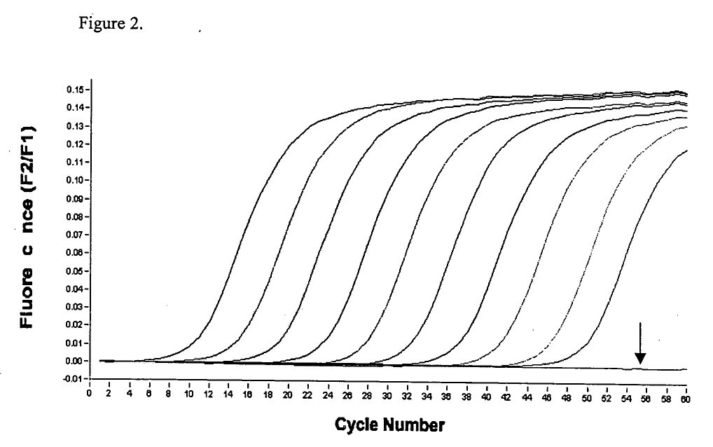

FIG. 2 shows the PCR amplification curve of pGEM-BZLF1 serial log dilutions ranging from 10⁹ to 1 copy per reaction and a water negative control (indicated by the arrow). As the input target copy number decreases, the cycle number at which exponential amplification occurs increases.

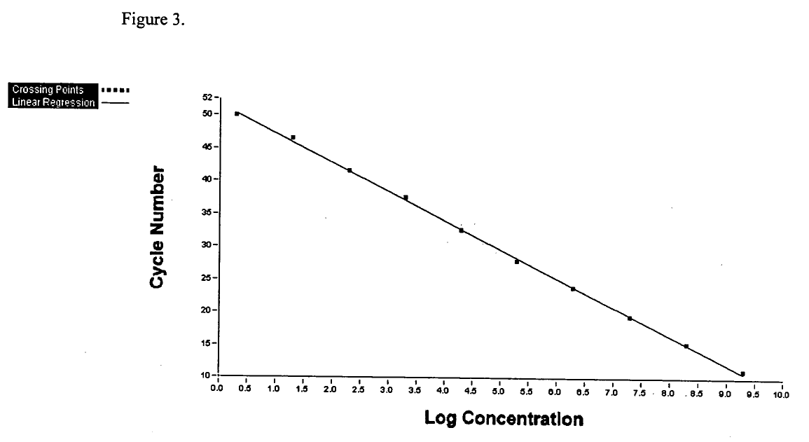

FIG. 3 shows a standard curve representing a serial log dilution of pGEM-BZLF1 standards. Each point indicates a log dilution from 10⁹ to 1 copy per reaction plotted against the logarithm of concentration. These gave an error value of 0.09 and a linear regression value (r²) of −1.00.

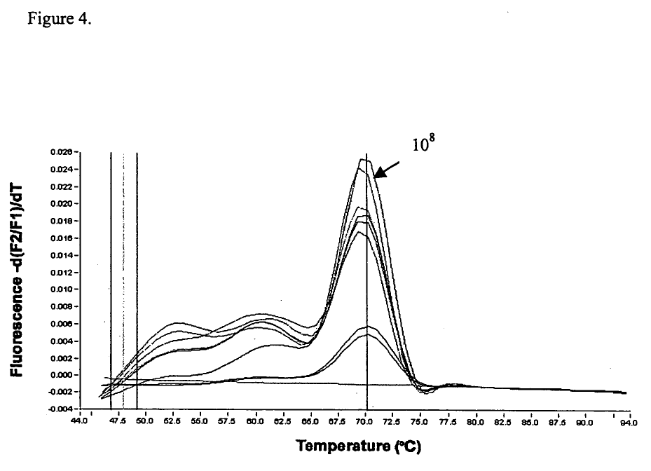

FIG. 4 shows the melt curve analysis of the 10⁸ pGEM-BZLF1 external standard at 70° C. (indicated by the arrow). The other melt peaks are derived from positive extracted EDTA blood samples and a water negative control (no melt peak).

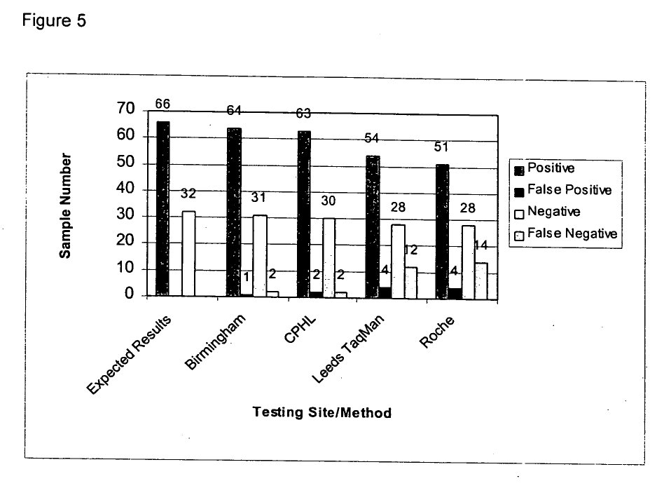

FIG. 5 shows results of a comparative assay carried out using the method of the invention at two different test sites (Birmingham and CPHL), the known Roche EBV DNA real time PCR assay, and the known Leeds TaqMan assay. Positive, false positive, negative and false negative results are shown for each test.

FIG. 6 shows conventional sequence alignment methodology as applied to the present invention. In more detail, FIG. 6 shows a nucleotide sequence alignment of EBV BZLF1 (query sequence) and the sequence of the BZLF 1 homologue from African Green Monkey herpes virus (subject sequence).

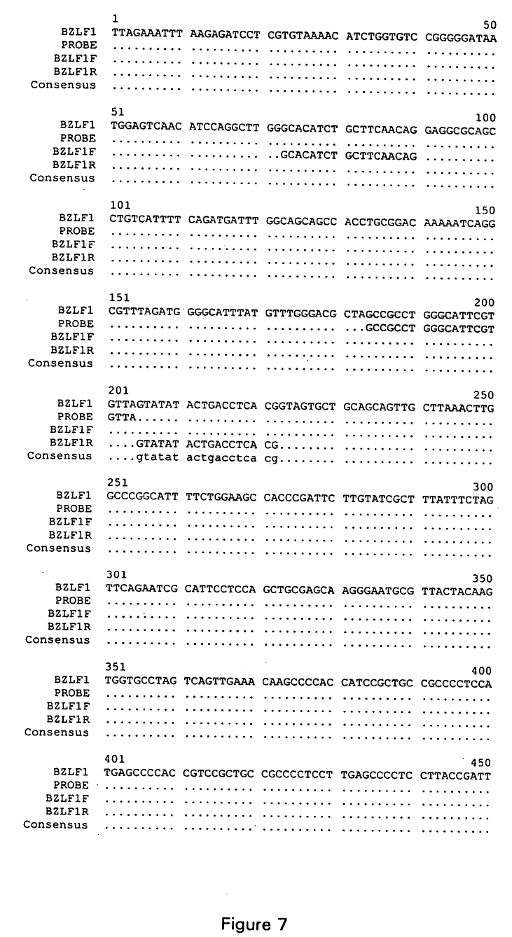

FIG. 7 shows the region of the BZLF 1 gene sequence to which, in a specific embodiment, the forward and reverse primers and the probe bind. Only nucleotides 1-450 of the BZLF gene are shown. In this embodiment, the forward primer (BZLF1F) is SEQ ID NO. 4 and binds to the complementary strand of SEQ ID NO. 1 between nucleic acid residues 72-90 (based on the numbering of the nucleic acids of SEQ ID NO. 1). The reverse primer (BZLF1R) is SEQ ID NO. 6 and binds to SEQ ID NO. 1 between nucleic acid residues 205-222 (in this Figure, the reverse primer is, however, written as its reverse compliment). The probe is SEQ ID NO. 2 and binds to SEQ ID NO. 1 between nucleic acid residues 184-204.

### REFERENCES

The teachings of all references cited are incorporated herein by reference.

Cockfield, S. M., Preiksaitis, J. K., Jewell, L. D. and Parfrey, N. A. (1993) Transplantation 56(1), 88-96.

Ellis, D., Jaffe, R., Green, M., Janosky, J. J., Lombardozzi-Lane, S., Shapiro, R., Scantlebury, V., Vivas, C. and Jordan, M. L. (1999) Transplantation 68(7), 997-1003.

Fischer, A., et al. (1991) N Engl J Med 324(21), 1451-6.

Henle, W. and Henle, G. (1981) Cancer Res 41(11 Pt 1), 4222-5.

Hsieh, W. S., Lemas, M. V. and Ambinder, R. F. (1999) Transpl Infect Dis 1(3), 204-12.

Kimura, H., Morita, M., Yabuta, Y., Kuzushima, K., Kato, K., Kojima, S., Matsuyama, T. and Morishima, T. (1999) J. Clin. Microbiol. 37(1), 132-136.

Niesters, H. G. M., van Esser, J., Fries, E., Wolthers, K. C., Cornelissen, J. and Osterhaus, A. D. M. E. (2000) J. Clin. Microbiol. 38(2), 712-715.

Swinnen, L., Costanzo-Nordin, M., Fisher, S., O'Sullivan, E., Johnson, M., Heroux, A., Dizikes, G., Pifarre, R. and Fisher, R. (1990) N Engl J Med 323(25), 1723-1728.

Walker, R. C., Marshall, W. F., Strickler, J. G., Wiesner, R. H., Velosa, J. A., Habermann, T. M., McGregor, C. G. and Paya, C. V. (1995a) Clin Infect Dis 20(5), 1346-53.

Walker, R. C., Paya, C. V., Marshall, W. F., Strickler, J. G., Wiesner, R. H., Velosa, J. A., Habermann, T. M., Daly, R. C. and McGregor, C. G. (1995b) J Heart Lung Transplant 14(2), 214-21.

### EXAMPLES

Culture of EBV Cell Line

The Namalwa EBV cell line (ATCC 1432, USA) containing 2 copies of the EBV genome per cell, was used as the source of DNA for assay development. Cell culture was performed according to ATCC guidelines. Cells were harvested by centrifugation for 15 mins at 500 g and the supernatant removed. Total viral DNA was extracted using an Easy-DNA kit (Invitrogen, Netherlands) according to the manufacturer's instructions. DNA recovery was confirmed by agarose gel electrophoresis.

BZLF1 Primer and Probe Design

A 150 bp region of BZLF1 was targeted for amplification using the following primers whose specificity was confirmed by BLAST database analysis (http://www.ncbi.nlm.nih.gov/BLAST/). The forward (BZLF1F) and reverse primers (BZLF1R) used in this example were 5′ GCACATCTGCTTCAACAG 3′ and 5′ CGTGAGGTCAGTATATAC 3′ respectively. The reverse primer was labelled with LCRed 640 at nucleotide residue 16. A sequence specific probe, 3′ end labelled with fluorescein, was used —of sequence 5′ GCCGCCTGGGCATTCGTGTTA 3′ in this example. All primers and probes were synthesised by TIB Mol Biol (TIB Mol Biol, Germany).

Quantitative LightCycler PCR

PCR reactions were carried out in a final volume of 20 μl using FastStart DNA Master Hybridisation Probes mix (Roche Diagnostics GmBH, Germany). Forward and reverse primers were used at 2 μM and 5 μM respectively, the probe at 1 μM and magnesium chloride adjusted to a final concentration of 4 mM. The above were prepared as a mastermix of which 18 μl were transferred into glass capillaries together with 2 μl of relevant sample DNA. Cycling conditions were as follows: 95° C. for 10 mins followed by 60 cycles of 95° C. 2 s, 55° C. 5 s and 72° C. 6 s with transition rates of 20° C./s, 20° C./s and 2° C./s respectively with a single fluorescence acquisition at 55° C. and a single melt cycle of 95° C. for 0 s, 40° C. 20 s, 99° C. 0 s with transition rates of 20° C./s, 5° C./s and 0.2° C./s respectively with a continuous fluorescence acquisition.

Sequencing

The amplicon was excised from a 1.2% agarose gel under UV illumination, purified using the QIAquick PCR Purification kit (Qiagen Ltd, UK). The purified product was ligated and transformed into a pGEM-T Vector system II (Promega, Wis., USA) according to the manufacturer's protocol. DNA was extracted from recombinant colonies using a Wizard Plus SV DNA miniprep kit (Promega, Wis., USA). Product specificity was confirmed by sequencing using 5′ end-labelled CY5.5 and CY5 primers (Sigma-Genosys Ltd, UK) complementary to the T7 and SP6 regions of the vector respectively. The construct was termed pGEM-BZLF1.

BZLF1 Quantitation

pGEM-BZLF1 was linearised with Sal1 (Promega, Wis., USA) according to the manufacturer's protocol, subjected to preparative gel electrophoresis and purified using a QIAquick PCR Purification kit (Qiagen Ltd, UK). DNA was subjected to analytical gel electrophoresis together with a 50 bp smart ladder (Oswel Research Products, UK) and quantified using UVI Doc Image Analysis Software (UVItec Ltd, UK). A set of serial log dilutions ranging from 10⁹ to 1 copy containing 5 ng/μl of herring sperm carrier DNA (Promega, Wis., USA) were prepared to assess the sensitivity of the assay. No cross-reactivity was found using samples containing CMV, VZV, HSV1 or HSV2 DNA. The EBV negative cell line, RAMOS, was used to confirm the specificity of the primers and probe.

Assay Standardisation

For assay standardisation, serial log dilutions of EBV DNA were prepared ranging from 1×10⁴ to 1 copy (EBV B95-8, Advanced Biotechnologies Inc, UK). Intra- and inter-assay variation studies were carried out in triplicate to determine the reproducibility of the LightCycler assay.

Viral DNA Extraction from Blood

Viral DNA was extracted from EDTA whole blood and plasma samples with a QIAamp DNA Blood mini kit according to the manufacturer's instructions (Qiagen Ltd, UK).

Results

Melt Curve Analysis

Typical results from the probe melt curve analysis are shown in FIG. 1. The melting temperature (Tm) of the BZLF1 probe was approximately 70° C. All products were subjected to analytical gel electrophoresis to confirm the 150 bp amplicon size.

Quantitation

Using the ‘Quantitation Analysis’ program, the cycle at which reactions enter the log/linear phase is detected and distinguished from background fluorescence to determine initial target concentration (FIG. 2). The crossing point of each standard was measured and plotted against the logarithm of concentration to produce a standard curve resulting in an error of less than 1.0 with a linear regression value of −1.00 indicating good correlation between the ranges used (FIG. 3). Unknown sample copy numbers are then extrapolated and quantified from the standard curve.

Assay Standardisation

Intra- and inter-assay variation studies were carried out by testing the pGEM-BZLF1 external standards in triplicate. The log₁₀ differences were calculated and shown to be within a 0.5 log₁₀ range (see Table 1 below). A 0.6 log difference was seen at both the lower and higher ends of the inter-assay variation study.

TABLE 1
Log₁₀ differences seen on testing the quantitative standards.

| Intra-assay | Log₁₀ | | Log₁₀ |
|---|---|---|---|
| variation | difference | Inter-assay variation | difference |
| 2.0 − 2.3 × 10⁸ | 0.1 | 1.2 − 4.4 × 10⁸ | 0.6 |
| 1.8 − 1.6 × 10⁷ | 0.1 | 1.5 − 2.0 × 10⁷ | 0.1 |
| 2.4 − 2.7 × 10⁶ | 0.1 | 1.4 − 1.6 × 10⁶ | 0.1 |
| 1.7 − 2.0 × 10⁵ | 0.1 | 1.3 − 1.5 × 10⁵ | 0.1 |
| 1.4 − 2.6 × 10⁴ | 0.3 | 1.4 − 1.7 × 10⁴ | 0.1 |
| 1.3 − 1.9 × 10³ | 0.2 | 1.3 − 2.7 × 10³ | 0.3 |
| 1.1 − 1.7 × 10² | 0.2 | 1.4 − 1.8 × 10² | 0.1 |
| 1.2 − 1.6 × 10¹ | 0.1 | 1.3 − 4.8 × 10¹ | 0.3 |
| 1.2 − 3.5 × 10⁰ | 0.5 | 2.4 − 4.1 × 10⁰ | 0.2 |
| 2.3 × 10⁻¹ − 1.0 × 10⁰ | 0.6 | 3.1 − 7.3 × 10⁻¹ | 0.4 |

Blood Samples

EBV DNA was detected in extracted whole blood and plasma samples. FIG. 4 shows a probe Tm of 70° C. identical to 10⁸ pGEM-BZLF1 external standard in whole blood and plasma extracts taken from the same patient at various time intervals. These were calculated to be between 10⁸ and <500 copies per ml. As part of the study investigating intra- and inter-assay variation, tests were carried out in triplicate using 8 clinical samples (see Table 2 below). All the differences were within a 0.5 log₁₀ range.

TABLE 2
Log₁₀ differences on testing a set of clinical samples

| | Intra-assay | Log₁₀ | Inter-assay | Log₁₀ |
|---|---|---|---|---|
| Sample No. | variation | difference | variation | difference |
| 1 | 3.7 − 2.2 × 10¹ | 0.3 | 3.1 − 2.3 × 10¹ | 0.2 |
| 2 | 3.3 − 1.6 × 10¹ | 0.3 | 1.6 − 1.3 × 10¹ | 0.1 |
| 3 | 6.0 − 2.3 × 10⁰ | 0.4 | 7.0 − 4.6 × 10⁰ | 0.1 |
| 4 | 6.3 − 1.9 × 10⁰ | 0.5 | 2.3 − 1.0 × 10⁰ | 0.4 |
| 5 | 6.1 − 3.8 × 10⁵ | 0.2 | 9.4 − 8.9 × 10⁵ | 0.1 |
| 6 | 3.6 − 3.5 × 10² | 0.1 | 4.0 − 3.8 × 10² | 0.02 |
| 7 | 2.5 − 2.3 × 10¹ | 0.03 | 4.1 − 1.7 × 10¹ | 0.4 |
| 8 | 3.2 − 2.5 × 10² | 0.1 | 3.0 − 2.8 × 10² | 0.1 |

Comparative Example

The reagents were sent for Beta-Testing to three other Public Health Laboratories, at Birmingham, Leeds and the Central Public Health Laboratory (CPHL) at Colindale, together with 66 positive samples and 32 negative samples (see FIG. 5).

The CPHL also tested the samples using the Roche LightCycler EBV assay (ref: Development of a quantitative LightCycler assay for the detection of Epstein-Barr virus DNA in research samples (2002) Biochimica: No 3.). Neither the gene, probe or primer sequences used in the Roche assay are provided. Roche state that although the quantitation standards range from 10² to 10⁶ copies/reaction, the assay has a dynamic detection range of 10 to 10¹⁰ copies/reaction.

The Leeds laboratory tested our samples with their in-house TaqMan PCR assay. This PCR-based analysis permits real-time detection, but is run on a solid block machine and is, therefore, slower than using the LightCycler system (˜3 hours vs. 45 minutes). Furthermore, the TaqMan system does not allow for melt-curve analysis, an analysis that can assist in interpreting questionable positives.

The Roche assay performed poorly in comparison to the present assay, picking up only 77% of the positive samples, although it fared slightly better with the negative samples, identifying 88% correctly (see Table 3 below and FIG. 5). The majority of the positives missed by the Roche assay were less than/equal to approximately 1000 copies/reaction. This suggests that the assay may not be as sensitive as Roche claim. The Leeds TaqMan assay was slightly better than the Roche assay, although it only picked up 82% of the positive and again 88% of the negative samples.

TABLE 3
Percentage of positive & negative samples scored from each of
the testing sites, using either the present assay (LightCycler
embodiment), the TaqMan assay or the Roche LightCycler
assay.

| | | Birmingham | | | |
|---|---|---|---|---|---|
| | | present | CPHL present | | |
| | | invention | invention | | |
| | Expected | LightCycler | LightCycler | TaqMan | Roche |
| | result | embodiment | embodiment | assay | assay |
| Positive | 68 | 64 * | 63 # | 54 * | 51 # |
| | | (97%) | (97%) | (82%) | (78%) |
| Negative | 32 | 31 | 30 | 28 | 28 |
| | | (97%) | (94%) | (88%) | (88%) |

* 2 positive samples missing
# 3 positive samples missing

DNA Aspect of the Present Invention

1) Preparing the Probes

Probes of the present invention may be prepared by PCR amplifying a target region of BZLF1 (and optionally other genes) through the use of a pair of primers (forward and reverse), which results in PCR products of approximately 200 base pairs.#

Alternatively, the target regions selected as probes may be chemically synthesised to form short (20-80 bases) oligonucleotides. The advantage of the oligonucleotide method is that it saves a considerable amount of time spent on performing/optimising PCRs.

The probe preparation is then purified, for example by ethanol or isopropanol precipitation or using PCR/oligo purification kits, e.g., Qiagen, Millipore or TelChem.

2) Slide Surface Chemistries

There are a number of different approaches to binding DNA to glass slides, but all can be divided into two groups: hydrogen bonding or covalent linkage.

Hydrogen Bonding

This can occur between amine groups coated on the slide and phosphate groups in the DNA backbone. The slides can be coated with either Poly-L-lysine or aminosilane to give an amine group-coated slide. Once the DNA is spotted onto the slide, UV cross-linking or oven baking allows some covalent bonds to be formed between the amine groups and the DNA. The remaining free amine groups on the slide are then blocked. This is achieved by treatment with succinic anhydride (a molecule with two active carboxyl groups). A condensation reaction takes place and for every succinic anhydride molecule two peptide bonds are formed with the Poly-L lysine/amino silane.

Covalent bonding

Covalent bonding can occur between active groups (such as aldehyde, epoxy, or isothiocyanate) coating the slide and amine modified DNA. In this regard, amine groups are preferably synthetically attached to the PCR primers during the synthesis of the oligonucleotides. Accordingly, the PCR primers are then be used to amplify the cDNAs giving the amine modified DNA.

Slides can be prepared by treating aminosilane slides with phenyleridiisothiocyanate. Alternatively, activated slides can be purchased (3D-Link activated slides, Surmodics; Silylated slides, CSS-25, TeleChem). These active slides are moisture sensitive and only once the target DNA has been spotted onto them can they be exposed to moisture. This then deactivates the groups to prevent unwanted probe binding to the slide.

3) Diagnostic Analysis

This can be divided into two types of analysis. First, to detect the presence of EBV, and secondly to monitor viral load (amount of virus in sample).

For simple detection, DNA is isolated from a clinical sample (whole EDTA bloods, lymph-node tissue, serum), using Qiagen Blood/tissue kits and amplified by PCR incorporating a fluorescent dye. This amplification may be directed to a number of gene targets.

For a quantitative analysis, RNA is extracted, reverse transcribed and fluorescently-labelled. In this regard, RNA may be extracted by using Rneasy kits (Qiagen), High Pure RNA isolation kits (Roche) or more traditionally using Trizol (Gibco BRL). Some workers recommend using both a kit and Trizol to produce good quality RNA. No PCR amplification is required. In this regard, fluorescently labelling the cDNA from the RNA may be carried out using commercially available reagents and dyes, or kit based methods, e.g., CyScribe Post-Labelling (Amersham). The labelled cDNA may be then applied to a DNA array for quantitative analysis. Such an array would comprise a selected probe for the BZLF1 gene target (optionally with other probes), together with predetermined quantitative spots of colour fixed to the array, which spots represent serially diluted quantities of DNA. The fluorescence intensity of the sample DNA bound to the slide is then compared with the serial standard spots and quantified.

To analyse a patient's response to therapy, RNA is extracted from the sample, copied into fluorescently labelled cDNA as described above and hybridised to the array. The DNA array comprises probes for informative gene targets (ie. BZFL1, and optionally other targets), the expression of which alters in response to a given therapy. Additional, invariant ‘house-keeping genes’ may be included in the array as controls. In this regard, the expression of the housekeeping genes should remain constant and such genes therefore provide good controls for monitoring extraction and cDNA labelling efficiencies. In contrast, variations in the informative gene target expression profiles may be used to infer successful/failing treatment, and the therapeutic regime amended accordingly. Levels of gene expression may be measured as relative changes in level, compared to a previous baseline figure, or to the house-keeping genes, or could be absolute, compared to serial spot dilutions as described above.

4) Using the DNA Array

Hybridisation

Hybridisations are carried out under a sealed coverslip in a humid chamber. Geneframes or coverslips sealed with Cow gum can be used to hybridise in a humid box in an oven.

Alternatively, Corning or TeleChem International hybridisation chambers can be used in a waterbath. These chambers are preferred, as the seal does not touch the coverslip or slide, and humidification can be ensured. The coverslip used should just cover the area of the microarray, and the volume of hybridisation adjusted accordingly. Care must be taken to ensure that no air bubbles are introduced under the coverslip when setting up hybridisations. Hybridisations can be carried out at either at 65° C. or 42° C. (if 50% formamide is included). General and species-specific blocking elements should be included in the hybridisation.

For human RNA derived probes use 10 μl of 1 mg/ml CoT-1 DNA (GIBCO/BRL) which blocks repetitive human DNA, yeast tRNA (GIBCO/BRL) which acts as a non-specific hybridisation blocker and poly dA (Amersham Pharmacia Biotech) which blocks the oligo dT. The CoT-1 DNA and the tRNA can be added to the probe just prior to purification.

Slide Washing

Hybridisation components such as SDS and SSC fluoresce. and, unless washed from the slide surface prior to scanning, will cause diffuse low-level background. This background can be minimised by immediately spin-drying slides by low-velocity centrifugation after washing. All washes should be performed at room temperature.

Slide Scanning, Signal Quantification and Data Analysis

The expression levels of different genes on the microarray are visualised by confocal laser scanning of the hybridised slide. There are a number of different scanners on the market, which detect Cy3, and Cy5 (or their spectral homologues) and at least one manufacturer—GSI Lumonics—produces instruments that can detect additional fluors. It is useful to scan slides at every stage of the process, to check on the background level of fluorescence on the slides and to check that the DNA has been bound effectively (by analysing the intensity of the landmark spots).

Slides can be scanned at several different laser intensities in order to ensure that all levels of expression are detected and that the spot intensity is not saturated. Image analysis software packages extract data from TIFF files generated from scanning the slide. In general, a circle is drawn around each spot and the integrated or mean intensity of signal within the spot is calculated. Background signal intensity data is also extracted together with standard deviation errors. These data can be exported in a tab-delineated format and imported into a spreadsheet packages (such as excel) or into the data mining packages. Composite images can also be generated where two images from the same slide are shown as one. Both the image analysis software and spreadsheet packages are capable of giving a graphical presentation of the data. The data mining packages look for trends and patterns in the data from a set of several slides. Various software packages are available for data extraction and analysis. These include:

For Image Analysis:

ArrayVision (Imaging Research),

Imagene (BioDiscovery),

AutoGene (BioDiscovery),

GenePix (Axon),

QuantArray (GSI Lumonics).

Data Mining:

GeneSight (BioDiscovery),

GeneSpring (Silicon Genetics),

Spotfire Pro (Spotfire).

1. A method for detection of Epstein Barr virus nucleic acid in an isolated sample, comprising:
(i) contacting said sample with a probe wherein the probe binds to a target region defined by SEQ. ID NO. 1 or its homologue or a complementary strand thereof, which binding provides a detectable signal, and
(ii) detecting said signal.

2. A method according to claim 1, further comprising the step of amplifying Epstein Barr virus nucleic acid prior to detecting said signal.

3. A method according to claim 2, wherein said amplifying step is carried out using a pair of primers, comprising forward and reverse oligonucleotide primers, the forward primer binding to a target site between nucleic acid residues 1-200, preferably 1-100, of the complementary strand of SEQ. ID NO. 1. or its homologue, and the reverse primer binding to a target site between nucleic acid residues 1-500, preferably 100-300, of SEQ. ID NO. 1 or its homologue.

4. A method according to claim 1, wherein said probe binds to a target site between nucleic acid residues 1-500, preferably 1-300, of SEQ ID NO. 1 or its homologue or a complementary strand thereof.

5. A method according to claim 1 wherein said probe is an oligonucleotide probe.

6. A method according to claim 5 wherein said probe is 1-50 nucleotides long.

7. A method according to claim 6 wherein said probe is 10-30 nucleotides long.

8. A method according to claim 6 wherein said probe is 15-25 nucleotides long.

9. A method according to claim 5 wherein said probe is of sequence SEQ ID. NO. 2 or 3.

10. A method according to claim 1 wherein said detectable signal is a change in fluorescence.

11. A method according to claim 10 wherein the probe is fluorescently labelled.

12. A method according to claim 3 wherein said forward and reverse oligonucleotide primers are 1 to 50 nucleotides long.

13. A method according to claim 15 wherein said oligonucleotide primers are 10 to 30 nucleotides long.

14. A method according to claim 15 wherein said oligonucleotide primers are 15-25 nucleotides long.

15. A method according to claim 12 wherein said forward primer is of SEQ. ID NOs. 4 or 5 and said reverse primer is of SEQ. ID Nos. 6 or 7.

16. A method according to claim 3 wherein said reverse primer is fluorescently labelled.

17. A method according to claim 3 wherein said forward primer is fluorescently labelled.

18. A method according to claim 3 wherein both forward and reverse primers are fluorescently labelled.

19. A probe suitable for use in a method according to claim 1, wherein said probe binds to a target region defined by SEQ. ID NO. 1 or its homologue or a complementary strand thereof, and said binding provides a detectable signal.

20. A probe according to claim 19, wherein said probe binds to a target site between nucleic acid residues 1-500, preferably 1-300, of SEQ. ID. NO. 1 or its homologue or a complementary strand thereof.

21. A probe according to claim 19 which is an oligonucleotide probe.

22. A probe according to claim 21 comprising 1-50 nucleotides.

23. A probe according to claim 21 comprising 10-30 nucleotides.

24. A probe according to claim 21 comprising 15-25 nucleotides.

25. A probe according to claim 21 of sequence SEQ. ID NO. 2 or 3.

26. A probe according to claim 19, wherein said binding is detectable by detecting a change in fluorescence.

27. A probe according to claim 26, wherein said probe is fluorescently labelled.

28. A pair of oligonucleotide primers, comprising forward and reverse primers, for use in a method according to claim 3, said forward primer binding to a target site between nucleic acid residues 1-200, preferably 1-100, of the complementary strand of SEQ. ID. NO. 1 or its homologue, and said reverse primer binding to a target site between nucleic acid residues 1-500, preferably 100-300, of SEQ. ID NO. 1 or its homologue.

29. A pair of primers according to claim 28, each comprising 1 to 50 nucleotides.

30. A pair of primers according to claim 29, each comprising 10 to 30 nucleotides.

31. A pair of primers according to claim 29, each comprising 15 to 25 nucleotides.

32. A pair of primers according to claim 28, said forward primer of sequence SEQ. ID. NOs. 4 or 5 and said reverse primer of SEQ. ID. NOs. 6 or 7.

33. A pair of primers according to claim 28 wherein said forward primer and said reverse primer are fluorescently labelled.

34. A forward primer for use in a method according to claim 3, wherein said forward primer binds to a target site between nucleic acid residues 1-200, preferably 1-100, of the complementary strand of SEQ. ID NO. 1 or its homologue.

35. A forward primer according to claim 34, comprising 1 to 50 nucleotides.

36. A forward primer according to claim 35, comprising 10 to 30 nucleotides.

37. A forward primer according to claim 35, comprising 15 to 25 nucleotides.

38. A forward primer according to claim 34, of sequence SEQ. ID. NOs. 4 or 5.

39. A forward primer according to claim 34 wherein said forward primer is fluorescently labelled.

40. A reverse primer for a method according to claim 3, wherein said reverse primer binds to a target site between nucleic acid residues 1-500, preferably 100-300, of SEQ. ID NO. 1 or its homologue.

41. A reverse primer according to claim 40, comprising 1 to 50 nucleotides.

42. A reverse primer according to claim 41, comprising 10 to 30 nucleotides.

43. A reverse primer according to claim 41, comprising 15-25 nucleotides.

44. A reverse primer according to claim 40, of sequence SEQ. ID. NOs. 6 or 7.

45. A reverse primer according to claim 40 wherein said reverse primer is fluorescently labelled.

46. Use of a probe according to claim 19 in the manufacture of a composition for detecting Epstein Barr virus nucleic acid.

47. Use of a forward primer according to claim 34, in the manufacture of a composition for detecting Epstein Barr virus nucleic acid.

48. Use of a pair of primers according to claim 28 in the manufacture of a composition for detecting Epstein Barr virus nucleic acid.

49. A kit for detection of Epstein Barr virus nucleic acid comprising a probe according to claim 19 claims.

50. A kit for detection of Epstein Barr virus nucleic acid comprising a forward primer according to claim 34.

51. A kit for detection of Epstein Barr virus nucleic acid comprising a reverse primer according to claim 40.

52. A method of quantifying EBV viral load in a first isolated sample, comprising:
(i) contacting said first sample with a probe wherein the probe binds to a target region defined by SEQ. ID NO. 1 or its homologue or a complementary strand thereof, which binding provides a detectable signal, and detecting said signal; and
(ii) comparing the results obtained in step (i) with results obtained using a second, control sample having a known EBV viral load;
and thereby quantifying EBV viral load in the first isolated sample.

53. An in vitro method of monitoring drug efficacy for alleviating EBV infection or an EBV induced medical condition, comprising:
(i) contacting in vitro a first sample with a probe wherein the probe binds to a target region defined by SEQ. ID NO. 1 or its homologue or a complementary strand thereof, which binding provides a detectable signal, and detecting said signal, wherein said first sample has been isolated from a patient; and
(ii) contacting in vitro a second sample with a probe wherein the probe binds to a target region defined by SEQ. ID NO. 1 or its homologue or a complementary strand thereof, which binding provides a detectable signal, and detecting said signal, wherein said second sample has been isolated from a patient after commencement of drug therapy; and
(iii) comparing the results from (i) and (ii) and thereby confirming the efficacy of said drug.

54. A DNA array comprising an immobilised nucleic acid probe that binds to a target region defined by SEQ. ID NO. 1 or its homologue or a complementary strand thereof.

55. A DNA array, wherein the probe is defined according to claim 20.

56. A method of detecting Epstein Barr virus nucleic acid in an isolated sample, substantially as hereinbefore described with reference to the description and/or as shown in the Figures.

57. A forward primer substantially as hereinbefore described with reference to the description and/or as shown in the Figures.

58. A reverse primer substantially as hereinbefore described with reference to the description and/or as shown in the Figures.

59. A probe substantially as hereinbefore described with reference to the description and/or as shown in the Figures.

60. Use of a reverse primer according to claim 40, in the manufacture of a composition for detecting Epstein Barr virus nucleic acid.

61. A kit for detection of Epstein Barr virus nucleic acid comprising a pair of primers according to claim 28.
